SEQUENCE LISTING (42 sequences)

SEQ ID NO: 1 - protein, 1087 aa, Homo sapiens [sequence omitted]

SEQ ID NO: 2 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSIRCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 3 - protein, 570 aa, Homo sapiens
  VARIANT (197)..(197): Xaa = Unknown or Other
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNINNMWGPTRLPTCVSVFPLECPALPMIHNGHHTSENVGSIAPGLSVTYSCESGYLLVGEKIINCLSSGKWSAVPPTCEEAXCKSLGRFPNGKVKEPPILRVGVTANFFCDEGYRLQGPPSSRCVIAGQGVAWTKMPVCGGGGSGGGGSCVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCT

SEQ ID NO: 4 - DNA, 1711 bp, Homo sapiens
atttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtaccaggaggatacaaaattagaggctctacaccctacagacatggtgattctgtgacatttgcctgtaaaaccaacttctccatgaacggaaacaagtctgtttggtgtcaagcaaataatataaataatatgtgggggccgacacgactaccaacctgtgtaagtgttttccctctcgagtgtccagcacttcctatgatccacaatggacatcacacaagtgagaatgttggctccattgctccaggattgtctgtgacttacagctgtgaatctggttacttgcttgttggagaaaagatcattaactgtttgtcttcgggaaaatggagtgctgtcccccccacatgtgaagaggcacsctgtaaatctctaggacgatttcccaatgggaaggtaaaggagcctccaattctccgggttggtgtaactgcaaactttttctgtgatgaagggtatcgactgcaaggcccaccttctagtcggtgtgtaattgctggacagggagttgcttggaccaaaatgccagtatgtggcggaggtgggtcgggtggcggcggatcttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagccaaatgcacaagtactggctggatacctgctccgagatgtacct

SEQ ID NO: 5 - protein, 560 aa, Murine
  VARIANT (252)..(254): Xaa = Unknown or Other
  VARIANT (252)..(254): Optional Linker
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCXXXCVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCT

SEQ ID NO: 6 - protein, 560 aa, Murine
  VARIANT (252)..(254): Xaa = Unknown or Other
  VARIANT (252)..(254): Optional Linker
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCXXXCVAEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCT

SEQ ID NO: 7 - protein, 560 aa, Murine
  VARIANT (197)..(197): Xaa = unknown or other
  VARIANT (255)..(257): Xaa = unknown or other
  VARIANT (255)..(257): Optional Linker
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNINNMWGPTRLPTCVSVFPLECPALPMIHNGHHTSENVGSIAPGLSVTYSCESGYLLVGEKIINCLSSGKWSAVPPTCEEAXCKSLGRFPNGKVKEPPILRVGVTANFFCDEGYRLQGPPSSRCVIAGQGVAWTKMPVCXXXEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCT

SEQ ID NO: 8 - protein, 560 aa, Murine
  VARIANT (197)..(197): Xaa = unknown or other
  VARIANT (255)..(257): Xaa = unknown or other
  VARIANT (255)..(257): Optional Linker
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNINNMWGPTRLPTCVSVFPLECPALPMIHNGHHTSENVGSIAPGLSVTYSCESGYLLVGEKIINCLSSGKWSAVPPTCEEAXCKSLGRFPNGKVKEPPILRVGVTANFFCDEGYRLQGPPSSRCVIAGQGVAWTKMPVCXXXEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCT

SEQ ID NO: 9 - protein, 557 aa, Murine
  VARIANT (252)..(254): Xaa = Unknown or Other
  VARIANT (252)..(254): Optional Linker
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCXXXEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCT

SEQ ID NO: 10 - protein, 557 aa, Murine
  VARIANT (252)..(254): Xaa = Unknown or Other
  VARIANT (252)..(254): Optional Linker
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCXXXEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCT

SEQ ID NO: 11 - protein, 21 aa, Homo sapiens
  VARIANT (1)..(21): signal peptide
MPMGSLQPLATLYLLGMLVAS

SEQ ID NO: 12 - DNA, 72 bp, Homo sapiens
  misc_feature (1)..(72): signal peptide
atgcccatggggtctctgcaaccgctggccaccttgtacctgctggggatgctggtcgcttcctgcctcgga

SEQ ID NO: 13 - protein, 17 aa, Homo sapiens
  VARIANT (1)..(17): signal peptide
MGAAGLLGVFLALVAPG

SEQ ID NO: 14 - DNA, 60 bp, Homo sapiens
  misc_feature (1)..(60): signal peptide
atgggcgccgcgggcctgctcggggttttcttggctctcgtcgcaccgggggtcctcggg

SEQ ID NO: 15 - protein, 1025 aa, Mus musculus
MLTWFLFYFSEISCDPPPEVKNARKPYYSLPIVPGTVLRYTCSPSYRLIGEKAIFCISENQVHATWDKAPPICESVNKTISCSDPIVPGGFMNKGSKAPFRHGDSVTFTCKANFTMKGSKTVWCQANEMWGPTALPVCESDFPLECPSLPTIHNGHHTGQHVDQFVAGLSVTYSCEPGYLLTGKKTIKCLSSGDWDGVIPTCKEAQCEHPGKFPNGQVKEPLSLQVGTTVYFSCNEGYQLQGQPSSQCVIVEQKAIWTKKPVCKEILCPPPPPVRNGSHTGSFSENVPYGSTVTYTCDPSPEKGVSFTLIGEKTINCTTGSQKTGIWSGPAPYCVLSTSAVLCLQPKIKRGQILSILKDSYSYNDTVAFSCEPGFTLKGNRSIRCNAHGTWEPPVPVCEKGCQAPPKIINGQKEDSYLLNFDPGTSIRYSCDPGYLLVGEDTIHCTPEGKWTPITPQCTVAECKPVGPHLFKRPQNQFIRTAVNSSCDEGFQLSESAYQLCQGTIPWFIEIRLCKEITCPPPPVIHNGTHTWSSSEDVPYGTVVTYMCYPGPEEGVKFKLIGEQTIHCTSDSRGRGSWSSPAPLCKLSLPAVQCTDVHVENGVKLTDNKAPYFYNDSVMFKCDDGYILSGSSQIRCKANNTWDPEKPLCKKEGCEPMRVHGLPDDSHIKLVKRTCQNGYQLTGYTYEKCQNAENGTWFKKIEVCTVILCQPPPKIANGGHTGMMAKHFLYGNEVSYECDEGFYLLGEKSLQCVNDSKGHGSWSGPPPQCLQSSPLTHCPDPEVKHGYKLNKTHSAFSHNDIVHFVCNQGFIMNGSHLIRCHTNNTWLPGVPTCIRKASLGCQSPSTIPNGNHTGGSIARFPPGMSVMYSCYQGFLMAGEARLICTHEGTWSQPPPFCKEVNCSFPEDTNGIQKGFQPGKTYRFGATVTLECEDGYTLEGSPQSQCQDDSQWNPPLALCKYRRWSTIPLICGISVGSALIILMSVGFCMILKHRESNYYTKTRPKEGALHLETREVYSIDPYNPAS

SEQ ID NO: 16 - protein, 1249 aa, Mus musculus
MRLSARIIWLILWTVCAAEDCKGPPPRENSEILSGSWSEQLYPEGTQATYKCRPGYRTLGTIVKVCKNGKWVASNPSRICRKKPCGHPGDTPFGSFRLAVGSQFEFGAKVVYTCDDGYQLLGEIDYRECGADGWINDIPLCEVVKCLPVTELENGRIVSGAAETDQEYYFGQVVRFECNSGFKIEGHKEIHCSENGLWSNEKPRCVEILCTPPRVENGDGINVKPVYKENERYHYKCKHGYVPKERGDAVCTGSGWSSQPFCEEKRCSPPYILNGIYTPHRIIHRSDDEIRYECNYGFYPVTGSTVSKCTPTGWIPVPRCTLKPCEFPQFKYGRLYYEESLRPNFPVSIGNKYSYKCDNGFSPPSGYSWDYLRCTAQGWEPEVPCVRKCVFHYVENGDSAYWEKVYVQGQSLKVQCYNGYSLQNGQDTMTCTENGWSPPPKCIRIKTCSASDIHIDNGFLSESSSIYALNRETSYRCKQGYVTNTGEISGSITCLQNGWSPQPSCIKSCDMPVFENSITKNTRTWFKLNDKLDYECLVGFENEYKHTKGSITCTYYGWSDTPSCYERECSVPTLDRKLVVSPRKEKYRVGDLLEFSCHSGHRVGPDSVQCYHFGWSPGFPTCKGQVASCAPPLEILNGEINGAKKVEYSHGEVVKYDCKPRFLLKGPNKIQCVDGNWTTLPVCIEEERTCGDIPELEHGSAKCSVPPYHHGDSVEFICEENFTMIGHGSVSCISGKWTQLPKCVATDQLEKCRVLKSTGIEAIKPKLTEFTHNSTMDYKCRDKQEYERSICINGKWDPEPNCTSKTSCPPPPQIPNTQVIETTVKYLDGEKLSVLCQDNYLTQDSEEMVCKDGRWQSLPRCIEKIPCSQPPTIEHGSINLPRSSEERRDSIESSSHEHGTTFSYVCDDGFRIPEENRITCYMGKWSTPPRCVGLPCGPPPSIPLGTVSLELESYQHGEEVTYHCSTGFGIDGPAFIICEGGKWSDPPKCIKTDCDVLPTVKNAIIRGKSKKSYRTGEQVTFRCQSPYQMNGSDTVTCVNSRWIGQPVCKDNSCVDPPHVPNATIVTRTKNKYLHGDRVRYECNKPLELFGQVEVMCENGIWTEKPKCRGLFDLSLKPSNVFSLDSTGKCGPPPPIDNGDITSLSLPVYEPLSSVEYQCQKYYLLKGKKTITCTNGKWSEPPTCLHACVIPENIMESHNIILKWRHTEKIYSHSGEDIEFGCKYGYYKARDSPPFRTKCINGTINYPTCV

SEQ ID NO: 17 - protein, 559 aa, Mus musculus
ISCDPPPEVKNARKPYYSLPIVPGTVLRYTCSPSYRLIGEKAIFCISENQVHATWDKAPPICESVNKTISCSDPIVPGGFMNKGSKAPFRHGDSVTFTCKANFTMKGSKTVWCQANEMWGPTALPVCESDFPLECPSLPTIHNGHHTGQHVDQFVAGLSVTYSCEPGYLLTGKKTIKCLSSGDWDGVIPTCKEAQCEHPGKFPNGQVKEPLSLQVGTTVYFSCNEGYQLQGQPSSQCVIVEQKAIWTKKPVCKEILEDCKGPPPRENSEILSGSWSEQLYPEGTQATYKCRPGYRTLGTIVKVCKNGKWVASNPSRICRKKPCGHPGDTPFGSFRLAVGSQFEFGAKVVYTCDDGYQLLGEIDYRECGADGWINDIPLCEVVKCLPVTELENGRIVSGAAETDQEYYFGQVVRFECNSGFKIEGHKEIHCSENGLWSNEKPRCVEILCTPPRVENGDGINVKPVYKENERYHYKCKHGYVPKERGDAVCTGSGWSSQPFCEEKRCSPPYILNGIYTPHRIIHRSDDEIRYECNYGFYPVTGSTVSKCTPTGWIPVPRCT

SEQ ID NO: 18 - DNA, 1750 bp, Mus musculus
atgcccatggggtctctgcaaccgctggccaccttgtacctgctggggatgctggtcgcttccgtgctagcgatttcttgtgaccctcctcctgaagtcaaaaatgctcggaaaccctattattctcttcccatagttcctggaactgttctgaggtacacttgttcacctagctaccgcctcattggagaaaaggctatcttttgtataagtgaaaatcaagtgcatgccacctgggataaagctcctcctatatgtgaatctgtgaataaaaccatttcttgctcagatcccatagtaccagggggattcatgaataaaggatctaaggcaccattcagacatggtgattctgtgacatttacctgtaaagccaacttcaccatgaaaggaagcaaaactgtctggtgccaggcaaatgaaatgtggggaccaacagctctgccagtctgtgagagtgatttccctctggagtgcccatcacttccaacgattcataatggacaccacacaggacagcatgttgaccagtttgttgcggggttgtctgtgacatacagttgtgaacctggctatttgctcactggaaaaaagacaattaagtgcttatcttcaggagactgggatggtgtcatcccgacatgcaaagaggcccagtgtgaacatccaggaaagtttcccaatgggcaggtaaaggaacctctgagccttcaggttggcacaactgtgtacttctcctgtaatgaagggtaccaattacaaggacaaccctctagtcagtgtgtaattgttgaacagaaagccatctggactaagaagccagtatgtaaagaaattctcgaagattgtaaaggtcctcctccaagagaaaattcagaaattctctcaggctcgtggtcagaacaactatatccagaaggcacccaggctacctacaaatgccgccctggataccgaacacttggcactattgtaaaagtatgcaagaatggaaaatgggtggcgtctaacccatccaggatatgtcggaaaaagccttgtgggcatcccggagacacaccctttgggtcctttaggctggcagttggatctcaatttgagtttggtgcaaaggttgtttatacctgtgatgatgggtatcaactattaggtgaaattgattaccgtgaatgtggtgcagatggctggatcaatgatattccactatgtgaagttgtgaagtgtctacctgtgacagaactcgagaatggaagaattgtgagtggtgcagcagaaacagaccaggaatactattttggacaggtggtgcggtttgaatgcaattcaggcttcaagattgaaggacataaggaaattcattgctcagaaaatggcctttggagcaatgaaaagccacgatgtgtggaaattctctgcacaccaccgcgagtggaaaatggagatggtataaatgtgaaaccagtttacaaggagaatgaaagataccactataagtgtaagcatggttatgtgcccaaagaaagaggggatgccgtctgcacaggctctggatggagttctcagcctttctgtgaagaaaagagatgctcacctccttatattctaaatggtatctacacacctcacaggattatacacagaagtgatgatgaaatcagatatgaatgtaattatggcttctatcctgtaactggatcaactgtttcaaagtgtacacccactggctggatccctgttccaagatgtacct

SEQ ID NO: 19 - DNA, 2676 bp, Mus musculus
gaattcgccgccaccatgcccatggggtctctgcaaccgctggccaccttgtacctgctggggatgctggtcgcttccgtgctagcgatttcttgtgaccctcctcctgaagtcaaaaatgctcggaaaccctattattctcttcccatagttcctggaactgttctgaggtacacttgttcacctagctaccgcctcattggagaaaaggctatcttttgtataagtgaaaatcaagtgcatgccacctgggataaagctcctcctatatgtgaatctgtgaataaaaccatttcttgctcagatcccatagtaccagggggattcatgaataaaggatctaaggcaccattcagacatggtgattctgtgacatttacctgtaaagccaacttcaccatgaaaggaagcaaaactgtctggtgccaggcaaatgaaatgtggggaccaacagctctgccagtctgtgagagtgatttccctctggagtgcccatcacttccaacgattcataatggacaccacacaggacagcatgttgaccagtttgttgcggggttgtctgtgacatacagttgtgaacctggctatttgctcactggaaaaaagacaattaagtgcttatcttcaggagactgggatggtgtcatcccgacatgcaaagaggcccagtgtgaacatccaggaaagtttcccaatgggcaggtaaaggaacctctgagccttcaggttggcacaactgtgtacttctcctgtaatgaagggtaccaattacaaggacaaccctctagtcagtgtgtaattgttgaacagaaagccatctggactaagaagccagtatgtaaagaaattctcgaagattgtaaaggtcctcctccaagagaaaattcagaaattctctcaggctcgtggtcagaacaactatatccagaaggcacccaggctacctacaaatgccgccctggataccgaacacttggcactattgtaaaagtatgcaagaatggaaaatgggtggcgtctaacccatccaggatatgtcggaaaaagccttgtgggcatcccggagacacaccctttgggtcctttaggctggcagttggatctcaatttgagtttggtgcaaaggttgtttatacctgtgatgatgggtatcaactattaggtgaaattgattaccgtgaatgtggtgcagatggctggatcaatgatattccactatgtgaagttgtgaagtgtctacctgtgacagaactcgagaatggaagaattgtgagtggtgcagcagaaacagaccaggaatactattttggacaggtggtgcggtttgaatgcaattcaggcttcaagattgaaggacataaggaaattcattgctcagaaaatggcctttggagcaatgaaaagccacgatgtgtggaaattctctgcacaccaccgcgagtggaaaatggagatggtataaatgtgaaaccagtttacaaggagaatgaaagataccactataagtgtaagcatggttatgtgcccaaagaaagaggggatgccgtctgcacaggctctggatggagttctcagcctttctgtgaagaaaagagatgctcacctccttatattctaaatggtatctacacacctcacaggattatacacagaagtgatgatgaaatcagatatgaatgtaattatggcttctatcctgtaactggatcaactgtttcaaagtgtacacccactggctggatccctgttccaagatgtaccgaagattgtaaaggtcctcctccaagagaaaattcagaaattctctcaggctcgtggtcagaacaactatatccagaaggcacccaggctacctacaaatgccgccctggataccgaacacttggcactattgtaaaagtatgcaagaatggaaaatgggtggcgtctaacccatccaggatatgtcggaaaaagccttgtgggcatcccggagacacaccctttgggtcctttaggctggcagttggatctcaatttgagtttggtgcaaaggttgtttatacctgtgatgatgggtatcaactattaggtgaaattgattaccgtgaatgtggtgcagatggctggatcaatgatattccactatgtgaagttgtgaagtgtctacctgtgacagaactcgagaatggaagaattgtgagtggtgcagcagaaacagaccaggaatactattttggacaggtggtgcggtttgaatgcaattcaggcttcaagattgaaggacataaggaaattcattgctcagaaaatggcctttggagcaatgaaaagccacgatgtgtggaaattctctgcacaccaccgcgagtggaaaatggagatggtataaatgtgaaaccagtttacaaggagaatgaaagataccactataagtgtaagcatggttatgtgcccaaagaaagaggggatgccgtctgcacaggctctggatggagttctcagcctttctgtgaagaaaagagatgctcacctccttatattctaaatggtatctacacacctcacaggattatacacagaagtgatgatgaaatcagatatgaatgtaattatggcttctatcctgtaactggatcaactgtttcaaagtgtacacccactggctggatccctgttccaagatgtacctaa

SEQ ID NO: 20 - DNA, 2706 bp, Mus musculus
gaattcgccgccaccatgcccatggggtctctgcaaccgctggccaccttgtacctgctggggatgctggtcgcttccgtgctagcgatttcttgtgaccctcctcctgaagtcaaaaatgctcggaaaccctattattctcttcccatagttcctggaactgttctgaggtacacttgttcacctagctaccgcctcattggagaaaaggctatcttttgtataagtgaaaatcaagtgcatgccacctgggataaagctcctcctatatgtgaatctgtgaataaaaccatttcttgctcagatcccatagtaccagggggattcatgaataaaggatctaaggcaccattcagacatggtgattctgtgacatttacctgtaaagccaacttcaccatgaaaggaagcaaaactgtctggtgccaggcaaatgaaatgtggggaccaacagctctgccagtctgtgagagtgatttccctctggagtgcccatcacttccaacgattcataatggacaccacacaggacagcatgttgaccagtttgttgcggggttgtctgtgacatacagttgtgaacctggctatttgctcactggaaaaaagacaattaagtgcttatcttcaggagactgggatggtgtcatcccgacatgcaaagaggcccagtgtgaacatccaggaaagtttcccaatgggcaggtaaaggaacctctgagccttcaggttggcacaactgtgtacttctcctgtaatgaagggtaccaattacaaggacaaccctctagtcagtgtgtaattgttgaacagaaagccatctggactaagaagccagtatgtaaagaaattctcggcggaggtgggtcgggtggcggcggatctgaagattgtaaaggtcctcctccaagagaaaattcagaaattctctcaggctcgtggtcagaacaactatatccagaaggcacccaggctacctacaaatgccgccctggataccgaacacttggcactattgtaaaagtatgcaagaatggaaaatgggtggcgtctaacccatccaggatatgtcggaaaaagccttgtgggcatcccggagacacaccctttgggtcctttaggctggcagttggatctcaatttgagtttggtgcaaaggttgtttatacctgtgatgatgggtatcaactattaggtgaaattgattaccgtgaatgtggtgcagatggctggatcaatgatattccactatgtgaagttgtgaagtgtctacctgtgacagaactcgagaatggaagaattgtgagtggtgcagcagaaacagaccaggaatactattttggacaggtggtgcggtttgaatgcaattcaggcttcaagattgaaggacataaggaaattcattgctcagaaaatggcctttggagcaatgaaaagccacgatgtgtggaaattctctgcacaccaccgcgagtggaaaatggagatggtataaatgtgaaaccagtttacaaggagaatgaaagataccactataagtgtaagcatggttatgtgcccaaagaaagaggggatgccgtctgcacaggctctggatggagttctcagcctttctgtgaagaaaagagatgctcacctccttatattctaaatggtatctacacacctcacaggattatacacagaagtgatgatgaaatcagatatgaatgtaattatggcttctatcctgtaactggatcaactgtttcaaagtgtacacccactggctggatccctgttccaagatgtaccgaagattgtaaaggtcctcctccaagagaaaattcagaaattctctcaggctcgtggtcagaacaactatatccagaaggcacccaggctacctacaaatgccgccctggataccgaacacttggcactattgtaaaagtatgcaagaatggaaaatgggtggcgtctaacccatccaggatatgtcggaaaaagccttgtgggcatcccggagacacaccctttgggtcctttaggctggcagttggatctcaatttgagtttggtgcaaaggttgtttatacctgtgatgatgggtatcaactattaggtgaaattgattaccgtgaatgtggtgcagatggctggatcaatgatattccactatgtgaagttgtgaagtgtctacctgtgacagaactcgagaatggaagaattgtgagtggtgcagcagaaacagaccaggaatactattttggacaggtggtgcggtttgaatgcaattcaggcttcaagattgaaggacataaggaaattcattgctcagaaaatggcctttggagcaatgaaaagccacgatgtgtggaaattctctgcacaccaccgcgagtggaaaatggagatggtataaatgtgaaaccagtttacaaggagaatgaaagataccactataagtgtaagcatggttatgtgcccaaagaaagaggggatgccgtctgcacaggctctggatggagttctcagcctttctgtgaagaaaagagatgctcacctccttatattctaaatggtatctacacacctcacaggattatacacagaagtgatgatgaaatcagatatgaatgtaattatggcttctatcctgtaactggatcaactgtttcaaagtgtacacccactggctggatccctgttccaagatgtacctaa

SEQ ID NO: 21 - protein, 560 aa, Homo sapiens
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 22 - DNA, 1755 bp, Homo sapiens
gccgccaccatgggagccgctggtctgctcggcgtgttcctcgccttggtggcacctggcgtcctgggcatcagctgcggttcccctccaccaatcctgaatggcagaatctcctattactccacaccaatcgccgtcggcactgtgatcagatacagctgttcagggacttttcggctgatcggcgagaaaagcctcctctgcattaccaaggataaggtcgatgggacatgggataaaccagctcctaagtgcgagtacttcaataagtatagttcatgtccagagcccattgttcctggtggctacaagattcgggggagcacaccctatcgccacggtgactcagtgacctttgcttgtaaaaccaacttctcaatgaacggtaataagtcagtgtggtgtcaggccaataatatgtggggtcctacacgactccccacctgtgtgtccgtgttccccttggaatgccccgccctgcccatgatccataatggacaccacaccagcgagaatgtcgggagtatcgcacctggattgagtgtcacctactcatgcgagtctggctacctgcttgtaggtgaaaaaattattaattgcttgtcctccggcaaatggagtgccgttcccccaacttgtgaagaggcccggtgcaaatccctcggccgcttccctaatggtaaagttaaagagcctccaatcctcagagtgggggtgaccgctaacttcttctgtgatgaaggctaccggttgcagggaccacccagtagccggtgtgtcatagctgggcagggagtggcttggacaaagatgcccgtttgtgaggaaatcttcgaagactgtaatgagctgcccccaagacggaatacagagatcctcacaggctcttggtccgatcaaacttatccagagggtacccaggcaatttacaagtgcagacctggatacaggagcctgggcaatgtgattatggtgtgccgcaagggggagtgggtggcccttaatcctctccggaagtgtcagaaaagaccatgcggacaccctggagatacacctttcggtacctttacccttaccggcggcaatgtcttcgagtatggcgtcaaggccgtgtacacttgtaacgagggataccagctgctgggggaaataaactatcgtgagtgtgacactgacgggtggactaacgacatccccatttgcgaggtggtcaagtgccttcctgtaaccgctcccgaaaatggtaagatcgtatcttccgcaatggagcctgatcgggaataccactttggacaagccgttcggttcgtatgtaattcagggtataaaattgagggcgatgaggagatgcactgcagtgatgacggcttttggtcaaaggaaaagccaaagtgcgtagagatcagttgtaagtctcctgacgttattaacgggagtcccatcagtcagaagatcatttacaaggaaaacgagaggttccagtataaatgcaatatgggatatgagtactccgaaagaggggacgccgtgtgcacagagtccggatggcgacctttgccatcttgtgaagaaaagtcttgtgacaacccctatattcctaacggagattactctcctctgcgcatcaagcaccgaactggggacgagatcacttaccaatgtcgaaacggcttctaccctgctaccagaggtaacactgccaagtgtaccagcaccggttggattcccgcccccagatgcacacttaaatgataa

SEQ ID NO: 23 - protein, 863 aa, Homo sapiens
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 24 - DNA, 2665 bp, Homo sapiens
cgccgccaccatgggcgcagcaggcttgttgggcgtgttcctggcattggtggcacccggcgtattgggcatttcatgcggctctcctccacccattctcaatggaaggatctcctactacagcacccccatagctgtcggcaccgttatccgatacagttgttccggtactttccggcttatcggcgaaaagtctttgctgtgcattaccaaggataaagtggacgggacttgggacaaacccgcacctaagtgcgagtattttaacaaatatagcagctgccctgagcctatagtacccggggggtataaaatccggggctctactccctatcgtcatggcgattctgtgaccttcgcatgtaaaactaatttttcaatgaatggcaacaagtctgtatggtgtcaagcaaataacatgtggggacctacccgcctgccaacctgtgtgtcagtgtttcccctggaatgtccagccctccctatgatccacaacggacatcacaccagcgaaaacgttggatccatcgcaccagggctctctgtgacttactcttgcgagtccgggtacctgctcgtgggtgaaaagatcatcaactgcctcagtagtggtaaatggtccgccgtgcctcccacatgtgaagaggcccggtgcaagagcctgggccggttccccaacggaaaagtgaaggaacctcctatcttgagggttggtgtgaccgctaactttttctgcgacgaggggtacaggctccaagggcctccctctagtcggtgcgtaatcgccggtcaaggagtcgcatggactaagatgcctgtgtgtgaggagattttcgaggattgtaatgaattgccacccaggagaaatactgaaatcctgacaggctcttggtctgatcagacttatccagaaggcacccaggccatttacaagtgtcggcctggatacagatctctgggaaatgtgatcatggtatgtaggaaaggagagtgggtggctttgaaccccctccgcaagtgtcagaaaagaccatgcgggcatcctggagacaccccattcgggacatttacactgacaggcggaaacgtatttgagtacggagtcaaggccgtttatacatgtaacgaagggtatcaactgctgggagaaatcaactatagggagtgcgacactgacggatggacaaacgacattccaatctgcgaagtggtgaaatgtcttccagttacagcccctgaaaacgggaaaatcgtgtcctccgctatggagcctgaccgggaatatcatttcggccaggccgttagattcgtgtgtaatagcggctacaaaatcgagggcgacgaagaaatgcattgcagcgatgacgggttctggagcaaggagaagcctaaatgcgtcgaaatttcatgcaagagtcccgacgtcataaacggttctccaatttcccagaagatcatttataaggagaatgagcggttccagtataagtgtaatatgggctacgagtacagcgaacgcggtgacgccgtgtgtaccgaaagtggctggagaccactgcctagttgcgaggagaaatcctgcgacaacccttatattcccaacggggactactctcctctgagaatcaagcatcggactggcgacgagattacttaccaatgcaggaacggattctatccagcaactcggggcaataccgctaagtgtacctccacaggctggatacccgctcctagatgtacagaggactgcaatgaactgccacctcggcgcaatacagaaattttgactggatcatggtctgaccagacttaccccgagggcacccaggccatctacaaatgtaggcccggttatcgaagtttgggtaacgtgattatggtgtgtcgaaaaggtgaatgggtagcactcaatcccctccgtaaatgccagaagcgtccttgtgggcacccaggcgataccccttttggaactttcaccctgactggaggaaacgtctttgaatatggtgtgaaagccgtgtacacatgcaatgaagggtaccaactgctcggagagataaactatcgggagtgcgatacagatggatggaccaatgatataccaatctgcgaggtggtgaagtgtctcccagtcaccgctcctgagaacggaaagatcgtcagttctgctatggaacctgacagggaataccactttgggcaagccgtccgcttcgtgtgcaattcagggtacaagatagaaggcgacgaagagatgcactgttccgacgatggtttctggtctaaggagaagcctaaatgtgtcgagattagctgcaagtctcccgatgttattaacggctctcccatctctcaaaaaattatttataaggaaaacgaaagatttcagtacaagtgcaatatgggttatgagtacagtgaacgtggagacgccgtgtgcacagagtccgggtggcgtccactgcccagctgcgaagaaaaatcctgtgacaacccctacatccccaatggcgactattcccccctgcgcatcaaacatcgtactggcgatgaaattacttaccagtgccgcaacgggttctaccctgccacccggggtaacacagccaaatgcacctccaccggatggatccccgccccacgctgtaccttgaaatgatga

SEQ ID NO: 25 - protein, 20 aa, Homo sapiens
  VARIANT (1)..(20): signal peptide
MGAAGLLGVFLALVAPGVLG

SEQ ID NO: 26 - DNA, 60 bp, Homo sapiens
  misc_feature (1)..(60): signal peptide
atgggagccgctggtctgctcggcgtgttcctcgccttggtggcacctggcgtcctgggc

SEQ ID NO: 27 - protein, 7 aa, Homo sapiens
VSVFPLE

SEQ ID NO: 28 - protein, 4 aa, Homo sapiens
EEIF

SEQ ID NO: 29 - DNA, 20 bp, Artificial
  Primer
ttctgagtgtggtggtgagc

SEQ ID NO: 30 - DNA, 20 bp, Artificial
  Primer
agtccatggaaggtcacagg

SEQ ID NO: 31 - DNA, 20 bp, Artificial
  Primer
tcagataaggaggggcacaa

SEQ ID NO: 32 - DNA, 22 bp, Artificial
  Primer
atgaagaggtacccactctgga

SEQ ID NO: 33 - DNA, 20 bp, Artificial
  Primer
agcacagcagatgtgaatgc

SEQ ID NO: 34 - DNA, 20 bp, Artificial
  Primer
tttcttgcgctttcgttttt

SEQ ID NO: 35 - DNA, 20 bp, Artificial
  Primer
agctgagagggaaatcgtgc

SEQ ID NO: 36 - DNA, 19 bp, Artificial
  Primer
accagacagcactgtgttg

SEQ ID NO: 37 - DNA, 72 bp, Homo sapiens
atgcccatggggtctctgcaaccgctggccaccttgtacctgctggggatgctggtcgcttccgtgctagcg

SEQ ID NO: 38 - protein, 24 aa, Homo sapiens
MPMGSLQPLATLYLLGMLVASVLA

SEQ ID NO: 39 - DNA, 768 bp, Homo Sapiens
atttcttgtgaccctcctcctgaagtcaaaaatgctcggaaaccctattattctcttcccatagttcctggaactgttctgaggtacacttgttcacctagctaccgcctcattggagaaaaggctatcttttgtataagtgaaaatcaagtgcatgccacctgggataaagctcctcctatatgtgaatctgtgaataaaaccatttcttgctcagatcccatagtaccagggggattcatgaataaaggatctaaggcaccattcagacatggtgattctgtgacatttacctgtaaagccaacttcaccatgaaaggaagcaaaactgtctggtgccaggcaaatgaaatgtggggaccaacagctctgccagtctgtgagagtgatttccctctggagtgcccatcacttccaacgattcataatggacaccacacaggacagcatgttgaccagtttgttgcggggttgtctgtgacatacagttgtgaacctggctatttgctcactggaaaaaagacaattaagtgcttatcttcaggagactgggatggtgtcatcccgacatgcaaagaggcccagtgtgaacatccaggaaagtttcccaatgggcaggtaaaggaacctctgagccttcaggttggcacaactgtgtacttctcctgtaatgaagggtaccaattacaaggacaaccctctagtcagtgtgtaattgttgaacagaaagccatctggactaagaagccagtatgtaaagaaattctc

SEQ ID NO: 40 - DNA, 30 bp, Artificial
  Linker
ggcggaggtgggtcgggtggcggcggatct

SEQ ID NO: 41 - DNA, 957 bp, Mus musculus
tgcccagccccatcacagcttccttctgccaaacctataaatctaactgatgaatccatgtttcccattggaacatatttgttgtatgaatgtctcccaggatatatcaagaggcagttctctatcacctgcaaacaagactcaacctggacgagtgctgaagataagtgtatacgaaaacaatgtaaaactccttcagatcctgagaatggcttggtacatgtacacacaggcattcagtttggatcccgtattaattatacttgtaatcaaggataccgcctcattggttcctcctctgctgtatgtgtcatcactgatcaaagtgttgattgggatactgaggcacctatttgtgagtggattccttgtgagatacccccaggcattcccaatggagatttcttcagttcaaccagagaagactttcattatggaatggtggttacctaccgctgcaacactgatgcgagagggaaggcgctctttaacctggtgggtgagccctccttatactgtaccagcaacgatggtgaaattggagtctggagcggccctcctcctcagtgcattgaactcaacaaatgtactcctcctccctatgttgaaaatgcagtcatgctgtctgagaacagaagcttgttttccttaagggatattgtggagtttagatgtcaccctggctttatcatgaaaggagccagcagtgtgcattgtcagtccctaaacaaatgggagccagagttaccaagctgcttcaagggagtgatatgtcgtctccctcaggagatgagtggattccagaaggggttgggaatgaaaaaagaatattattatggagagaatgtaaccttggaatgtgaggatgggtatactctagaaggcagttctcaaagccagtgccagtctgatggcagctggaatcctcttctggccaaatgtgtatctcgctcaatc

SEQ ID NO: 42 - protein, 610 aa, Artificial
  Fusion Protein
  misc_feature (610)..(610): Xaa can be any naturally occurring amino acid
MPMGSLQPLATLYLLGMLVASVLAISCDPPPEVKNARKPYYSLPIVPGTVLRYTCSPSYRLIGEKAIFCISENQVHATWDKAPPICESVNKTISCSDPIVPGGFMNKGSKAPFRHGDSVTFTCKANFTMKGSKTVWCQANEMWGPTALPVCESDFPLECPSLPTIHNGHHTGQHVDQFVAGLSVTYSCEPGYLLTGKKTIKCLSSGDWDGVIPTCKEAQCEHPGKFPNGQVKEPLSLQVGTTVYFSCNEGYQLQGQPSSQCVIVEQKAIWTKKPVCKEILGGGGSGGGGSCPAPSQLPSAKPINLTDESMFPIGTYLLYECLPGYIKRQFSITCKQDSTWTSAEDKCIRKQCKTPSDPENGLVHVHTGIQFGSRINYTCNQGYRLIGSSSAVCVITDQSVDWDTEAPICEWIPCEIPPGIPNGDFFSSTREDFHYGMVVTYRCNTDARGKALFNLVGEPSLYCTSNDGEIGVWSGPPPQCIELNKCTPPPYVENAVMLSENRSLFSLRDIVEFRCHPGFIMKGASSVHCQSLNKWEPELPSCFKGVICRLPQEMSGFQKGLGMKKEYYYGENVTLECEDGYTLEGSSQSQCQSDGSWNPLLAKCVSRSIX

(19) United States
(12) Patent Application Publication
(10) Pub. No.: US 2019/0345516 A1
(43) Pub. Date: Nov. 14, 2019

(54) METHODS FOR TREATING EYE DISEASE

(71) Applicants: MUSC Foundation for Research Development, Charleston, SC (US); University of Utah Research Foundation, Salt Lake City, UT (US)

(72) Inventors: Monica Vetter, Salt Lake City, UT (US); Alejandra Bosco, Salt Lake City, UT (US); Baerbel Rohrer, Charleston, SC (US)

(21) Appl. No.: 16/351,289

(22) Filed: Mar. 12, 2019

Related U.S. Application Data
(60) Provisional application No. 62/641,594, filed on Mar. 12, 2018.

(51) Int. Cl. C12N 15/861; C07K 14/705; A61P 27/06

(52) U.S. Cl. CPC C12N 15/861; C07K 14/70596; C07K 2317/23; C07K 2319/00; A61P 27/06

(57) ABSTRACT

Recombinant vectors operably encoding a CR2-FH fusion protein comprising a CR2 portion comprising CR2 protein or a fragment thereof and a FH portion comprising a factor H protein or a fragment thereof, and pharmaceutical compositions comprising the recombinant vector, are described. Also provided are methods of using the compositions for treatment eye diseases such as macular degeneration or glaucoma.

### CROSS-REFERENCE TO RELATED APPLICATION

This application claims priority to U.S. Provisional Application Ser. No. 62/641,594, filed Mar. 12, 2018, the contents of which are incorporated by reference herein in their entirety.

### STATEMENT REGARDING FEDERALLY SPONSORED RESEARCH OR DEVELOPMENT

This invention was made with government support under EY020878, EY019320 and RX000444 awarded by the National Institutes of Health (NIH). The government has certain rights in the invention.

### BACKGROUND

Complement is the collective term for a series of blood proteins and is a major effector mechanism of the immune system. Complement plays an important role in the pathology of many autoimmune, inflammatory, and ischemic diseases, and is also responsible for many disease states associated with bioincompatibility. Inappropriate complement activation and its deposition on host cells can lead to complement-mediated cell lysis of target structures, as well as tissue destruction due to the generation of powerful mediators of inflammation.

Complement can be activated by one of the three pathways, the classical, lectin, and alternative pathways. The classical pathway is activated through the binding of the complement system protein Clq to antigen-antibody complexes, pentraxins, or apoptotic cells. The pentraxins include C-reactive protein and scrum amyloid P component. The lectin pathway is initiated by microbial saccharides via the man nose-binding lectin. The alternative pathway is activated on surfaces of pathogens that have neutral or positive charge characteristics and do not express or contain complement inhibitors. This is due to the process termed “tickover” of C3 that occurs spontaneously, involving the interaction of conformationally altered C3 with factor B, and results in the fixation of active C3b on pathogens or other surfaces. The alternative pathway can also be initiated when certain antibodies block endogenous regulatory mechanisms, by IgA-containing immune complexes, or when expression of complement regulatory proteins is decreased. In addition, the alternative pathway is activated by a mechanism called the “amplification loop” when C3b that is deposited onto targets via the classical or lectin pathway then binds factor B. Muller-Eberhard, 1988, Ann. Rev. Biochem. 57:321. For example, Holers and collaborators have shown that the alternative pathway is amplified at sites or local injury when inflammatory cells are recruited following initial complement activation. Girardi et al., J. Clin. Invest. 2003, 112:1644. Dramatic complement amplification through the alternative pathway then occurs through a mechanism that involves either the additional generation of injured cells that fix complement, local synthesis of alternative pathway components, or more likely because these infiltrating inflammatory cells that carry preformed C3 and properdin greatly increase activation specifically at that site.

Alternative pathway activation is imitated when circulating factor B binds to activated C3. This complex is then cleaved by circulating factor D to yield an enzymatically active fragment, C3bBb, C3bBb cleaves C3 generating C3b, which drives inflammation and also further amplifies the activation process, generating a positive feedback loop. Factor H (FH) is a key regulator (inhibitor) of the alternative complement pathway. It functions by competing with factor B for binding to C3b. Binding of C3b to Factor H also leads to degradation of C3b by factor 1 to the inactive form C3bi (also designated iC3b), thus exerting a further check on complement activation. The actual plasma concentration of factor H is approximately 500 μg/ml, providing complement regulation in the fluid phase, but its binding to cells is a regulated phenomenon that is enhanced by the presence of a negatively charged surface as well as fixed C3b, C3bi, or C3d, Jozsi et al., Histopathol (2004) 19:251-258.

The down-regulation of complement activation has been demonstrated to be effective in treating several disease indications in animal models and in ex vivo studies, e.g. systemic lupus erythematosus and glomerulonephritis (Y. Wang et al., Proc. Natl. Acad. Sci.; 1996, 93: 8563-8568), rheumatoid arthritis (Y. Wang et al., Proc. Natl. Acad. Sci., 1995; 92: 8955-8959), cardiopulmonary bypass and hemodialysis (C. S. Rinder, J. Clin. Invest., 1995; 96: 1564-1572), hypercute rejection in organ transplantation (T. J. Kroshus et al. Transplantation, 1995; 60: 1194-1202), myocardial infarction (J. W. Homeister et al., J. Immunol, 1993: 150: 1055-1064; H. F. Weisman et al., Science, 1990; 249: 146-151), reperfusion injury (E. A. Amsterdam et al., Am. J. Physiol., 1995; 268: H448-H457), and adult respiratory distress syndrome (R. Rabinovici et al., J. Immunol., 1992; 149: 1744-1750). In addition, other inflammatory conditions and autoimmune/immune complex diseases are also closely associated with complement activation (B. P. Morgan, Eur. J. Clin. Invest., 1994: 24: 219-228), including thermal injury, severe asthma, anaphylactic shock, bowel inflammation, urticaria, angioedema, vasculitis, multiple sclerosis, myasthenia gravis, membranoproliferative glomerulonephritis, and Sjogren's syndrome. Complement inhibitors and uses therefore are also disclosed in WO04/045520 and U.S. Pat. No. 6,521,450.

### SUMMARY OF THE INVENTION

This disclosure describes compositions and methods for treating eye disease, such as glaucoma and macular degeneration (MD), including age-related macular degeneration (AMD). More specifically, the disclosure describes recombinant polynucleotide vectors (referred to herein as “recombinant expression vectors” or simply “recombinant vectors”) which operably encode a CR2-FH fusion protein, and their use in gene therapy for treating eye disease. In some embodiments, the eye disease treated is one in which the alternative complement pathway is implicated.

In one aspect, the disclosure provides a recombinant vector, preferably a viral vector such as an adeno-associated virus (AAV) vector, which operably encodes a CR2-FH fusion protein. Examples of AAV vectors include AAV2, AAV5, AAV variant ShH10, and AAV6. Expression of the CR2-FH fusion protein encoded by the recombinant vector is under control of a promoter. Examples of suitable promoters include, without limitation, chicken β-actin (CBA) promoter, hSYN promoter, VMD2 promoter, RPE65 promoter, and OA1 promoter.

The CR2-FH fusion protein encoded by the recombinant vector comprises a) a CR2 portion comprising a complement receptor 2 (CR2) or a fragment thereof, and b) a FH portion comprising a Factor Horn fragment thereof. In some embodiments, the CR2-FH fusion protein encoded by the recombinant vector comprises a) a CR2 portion comprising at least the first two N-terminal SCR domains of CR2, and b) a FH portion comprising at least the first four N-terminal SCR domains of FH. In some embodiments, the CR2-FH fusion protein encoded by the recombinant vector comprises (and in some embodiments consists of or consists essentially of): a) a CR2 portion comprising the first four N-terminal SCR domains of CR2, and b) a FH portion comprising the first five N-terminal SCR domains of FH. In some embodiments, the CR2-FH fusion protein encoded by the recombinant vector comprises: a) a CR2 portion comprising a CR2 or a fragment thereof, and b) a FH portion comprising a FH or a fragment thereof, wherein the CR2-FH fusion protein is capable or binding to a CR2 ligand and wherein the CR2-FH fusion protein is capable of inhibiting complement activation of the alternative pathway. In some embodiments, the CR2-FH fusion protein encoded by the recombinant vector comprises (and in some embodiments consists of or consists essentially of): a) a CR2 portion comprising amino acids 23 to 271 of SEQ ID NO: 1, and b) a FH portion comprising amino acids 21 to 320 of SEQ ID NO:2. In some embodiments, the CR2-FH fusion protein encoded by the recombinant vector has an amino acid sequence of any or SEQ ID NO:3, SEQ ID NO:21, and SEQ ID NO:23. In some embodiments, the CR2-FH fusion protein encoded by the recombinant vector has an amino acid sequence that is at least about any of 50%, 60%, 70%, 80%, 90%, 91%, 92%, 93%, 94%, 95%, 96%, 97%, 98%, or 99% identical to that of any of SEQ ID NO:3, SEQ ID NO:21, and SEQ ID NO:23. In some embodiments, the CR2-FH fusion protein encoded by the recombinant vector comprises at least about 400, 450, 500, 550, or more contiguous amino acids of any of SEQ ID NO:3, SEQ ID NO:21, and SEQ ID NO:23. In some embodiments, the CR2-FH fusion protein encoded by the recombinant vector also comprises a signal peptide operably linked at the 5′ end of the sequence encoding the CR2-FH fusion protein. In some embodiments, a linker sequence is used for linking the CR2 portion and the FH portion. In some embodiments, the CR2-FH fusion protein encoded by the recombinant vector is encoded by a polynucleotide comprising a nucleic acid sequence of any of SEQ ID NO:4. SEQ ID NO:22, and SEQ ID NO:24. In some embodiments, the CR2-FH fusion protein encoded by the recombinant vector is encoded by a polynucleotide comprising a nucleic acid sequence that is at least about any of 50%, 60%, 70%, 80%, 90%, 91%, 92%, 93%, 94%, 95%, 96%, 97%, 98%, or 99% identical to that of any of SEQ ID NO:4, SEQ ID NO:22, and SEQ ID NO:24.

In another aspect, there is provided a pharmaceutical composition comprising the recombinant vector, preferably an adeno-associated virus (AAV) vector, which operably encodes a CR2-FH fusion protein; and a pharmaceutic ally acceptable carrier. In some embodiments, the pharmaceutical composition comprises a recombinant vector operably encoding a CR2-FH fusion protein comprising: a) a CR2 portion comprising a CR2 or a fragment thereof, and b) a FH portion comprising a FH or a fragment thereof: and a pharmaceutically acceptable carrier. In some embodiments, the pharmaceutical composition comprises a recombinant vector operably encoding a CR2-FH fusion protein comprising: a) a CR2 portion comprising at least the first two N-terminal SCR domains of CR2, and b) a FH portion comprising at least the first four N-terminal SCR domains of FH; and a pharmaceutically acceptable carrier. In some embodiments, the pharmaceutical composition comprises a recombinant vector operably encoding a CR2-FH fusion protein comprising (and in some embodiments consists of or consists essentially of): a) a CR2 portion comprising the first four N-terminal SCR domains of CR2, and b) a FH portion comprising the first five N-terminal SCR domains of FH; and a pharmaceutically acceptable carrier. In some embodiments, the pharmaceutical composition comprises a recombinant vector operably encoding a CR2-FH fusion protein comprising: a) a CR2 portion comprising a CR2 or a fragment thereof, and b) a FH portion comprising a FH or a fragment thereof, wherein the CR2-FH fusion protein is capable of binding to a CR2 ligand and wherein the CR2-FH fusion protein is capable of inhibiting complement activation of the alternative pathway: and a pharmaceutically acceptable carrier. In some embodiments, (the pharmaceutical composition comprises a recombinant vector operably encoding a CR2-FH fusion protein comprising (and in some embodiments consists of or consists essentially of): a) a CR2 portion comprising amino acids 23 to 271 of SEQ ID NO: 1, and b) a FH portion comprising amino acids 21 to 320 of SEQ ID NO:2; and a pharmaceutically acceptable earner. In some embodiments, the pharmaceutical composition comprises a recombinant vector operably encoding a CR2-FH fusion protein having on amino acid sequence of any of SEQ ID NO:3, SEQ ID NO:21, and SEQ ID NO:23; and a pharmaceutically acceptable carrier. In some embodiments, the pharmaceutical composition comprises a recombinant vector operably encoding a CR2-FH fusion protein having an amino acid sequence that is at least about 50%, 60%, 70%, 80%, 90%, 91%, 92%, 93%, 94%, 95%, 96%, 97%, 98%, or 99% identical to that of any of SEQ ID NO:3, SEQ ID NO. 21, and SEQ ID NO:23; and a pharmaceutically acceptable carrier. In some embodiments, the pharmaceutical composition comprises a recombinant vector operably encoding a CR2-FH fusion protein comprising at least about 400, 450, 500, 550, or more contiguous amino acids of any of SEQ ID NO:3, SEQ ID NO:21, and SEQ ID NO:23; and a pharmaceutically acceptable carrier. In some embodiments, the pharmaceutical composition comprises a recombinant vector operably encoding a CR2-FH fusion protein comprising a signal peptide operably linked at the 5′ end of the sequence encoding the CR2-FH fusion protein: and a pharmaceutically acceptable carrier. In some embodiments, the pharmaceutical composition comprises a recombinant vector operably encoding a CR2-FH fusion protein comprising a linker sequence for linking (the CR2 portion and the FH portion; and a pharmaceutically acceptable carrier. In some embodiments, the pharmaceutical composition comprises a recombinant vector operably encoding a CR2-FH fusion protein, wherein the CR2-FH fusion protein is encoded by a polynucleotide comprising a nucleic acid sequence of any of SEQ ID NO:4, SEQ ID NO:22, and SEQ ID NO:24; and a pharmaceutically acceptable carrier. In some embodiments, the pharmaceutical composition comprises a recombinant vector operably encoding a CR2-FH fusion protein, wherein the CR2-FH fusion protein is encoded by a polynucleotide comprising a nucleic acid sequence that is at least about any or 50%, 60%, 70%, 80%, 90%, 91%, 92%, 93%, 94%, 95%, 96%, 97%, 98%, or 99% identical to that of any of SEQ ID NO:4, SEQ ID NO:22, and SEQ ID NO:24; and a pharmaceutically acceptable carrier. In some embodiment, the pharmaceutical composition is suitable for delivery to the eye (Tor example by intraocular injection). In some embodiments, the pharmaceutical composition is suitable for intravenous injection.

In another aspect, the disclosure provides a method of treating eye disease, such as glaucoma or macular degeneration, in a subject, comprising administering to the subject an effective amount of a recombinant vector, or a pharmaceutical composition comprising a recombinant vector, wherein the recombinant vector operably encodes a CR2-FH fusion protein comprising: a) a CR2 portion comprising a CR2 or a fragment thereof, and b) a FH portion comprising a FH or a fragment thereof, including, without limitation, any of the CR2-FH fusion protein embodiments described herein, as well as CR2-FH variants as described herein. In some embodiments, the eye disease in one in which the alternative complement pathway is implicated. In some embodiments, the method comprises administering to a subject an effective amount of a recombinant vector, or a composition comprising a recombinant vector, wherein the vector operably encodes a CR2-FH fusion protein comprising: a) a CR2 portion comprising a CR2 or a fragment thereof, and b) a FH portion comprising a FH or a fragment thereof, wherein the CR2-FH fusion protein is capable of binding to a CR2 ligand and wherein the CR2-FH fusion protein is capable of inhibiting complement activation of the alternative pathway. In some embodiments, the recombinant vector operably encoding a CR2-FH fusion protein, or composition comprising the recombinant vector, is administered by intraocular injection. In some embodiments, the recombinant vector operably encoding a CR2-FH fusion protein, or composition comprising the recombinant vector, is administered by intravenous administration. It should be understood that treatment methods described herein are equally applicable to delivery of the recombinant vector to a subject irrespective of whether it is, or is not, provided as a component of a pharmaceutical composition.

In some embodiments, the eye disease to be treated is a disease that involves local inflammation. In some embodiments, the eye disease to be treated is a disease that is associated with FH deficiencies (including for example decrease in level of FH, decrease in activity of FH, or lacking wildtype or protective FH). In some embodiments, the eye disease to be treated is not a disease that is associated with FH deficiencies. In some embodiments, the eye disease to be treated is a drusen-associated disease. In some embodiments, the eye disease to be treated involves the classical complement pathway. In some embodiments, the eye disease to be treated does not involve the classical complement pathway.

In some embodiments, the eye disease to be treated is glaucoma. Accordingly, there is provided a method of treating glaucoma in subject comprising administering to the subject an effective amount of a recombinant vector that operably encodes a CR2-FH fusion protein, or an effective amount of a pharmaceutical composition comprising a recombinant vector that operably encodes a CR2-FH fusion protein, wherein the CR2-FH fusion protein comprises: a) a CR2 portion comprising a CR2 or a fragment thereof, and b) a FH portion comprising a FH or a fragment thereof including, without limitation, any of the CR2-FH fusion protein embodiments described herein, as well as CR2-FH variants as described herein. The recombinant vector for use in treating glaucoma is preferably an AAV2 vector, for example a YF mutant capsid AAV2 vector in which expression of the CR2-FH fusion protein is under the control of a chicken β-actin (CBA) promoter. In some embodiments, the recombinant AAV2 vector is delivered to the subject by way of intraocular injection, for example via intravitreal injection.

In some embodiments, the eye disease to be treated is macular degeneration (MD), including age-related macular degeneration (AMD). Accordingly, there is provided a method of treating macular degeneration in subject comprising administering to the subject an effective amount of a recombinant vector that operably encodes a CR2-FH fusion protein, or an effective amount of a pharmaceutical composition comprising a recombinant vector that operably encodes a CR2-FH fusion protein, wherein the CR2-FH fusion protein comprises: a) a CR2 portion comprising a CR2 or a fragment thereof, and b) a FH portion comprising a FH or a fragment thereof including, without limitation, any of the CR2-FH fusion protein embodiments described herein, as well as CR2-FH variants as described herein. The recombinant vector for use in treating macular degeneration may be an AAV5 vector, for example an AAV5 vector in which expression of the CR2-FH fusion protein is under the control of a VMD2 promoter. The recombinant vector for use in treating macular degeneration may bean AAV variant ShH10 or an AAV6 vector. In some embodiments, the recombinant AAV5 vector, recombinant AAV variant ShH10 or recombinant AAV6 vector is delivered to the subject by way of intraocular injection, for example via subretinal injection.

In another aspect, the disclosure provides a method for expressing a CR2-FH fusion protein in the eye of a subject at risk for or afflicted with glaucoma or macular degeneration. A recombinant adeno-associated virus (AAV) vector is delivered to the eye of a patient, for example via intravitreal injection or subretinal injection. The recombinant AAV vector operably encodes a CR2-FH fusion protein comprising: a) a CR2 portion comprising a CR2 or a fragment thereof, and b) a FH portion comprising a FH or a fragment thereof including, without limitation, any of the CR2-FH fusion protein embodiments described herein, as well as CR2-FH variants as described herein.

Other aspects or the disclosure provide a method of treating a subject at risk for or afflicted with glaucoma, the method comprising administering to the subject an effective amount of a recombinant adeno-associated virus (AAV) vector operably encoding a complement receptor 2 (CR2)-complement receptor 1 (CR1) fusion protein; wherein said CR2-CR1 fusion protein comprises a CR2 portion comprising a CR2 or a fragment thereof, and a CR1 portion comprising a CR1 or a fragment thereof; wherein the CR2 portion of the CR2-CR1 fusion protein is capable of binding to a CR2 ligand; and wherein the CR1 portion of the CR2-CR1 fusion protein is capable of inhibiting complement activation. In some embodiments, the recombinant adeno-associated virus vector is a recombinant AAV2 vector or a recombinant AAV5 vector. In some embodiments, the recombinant AAV vector comprises a chicken β-actin (CBA) promoter or a tissue-specific promoter for use in eye tissue. In some embodiments, the eye tissue comprises retinal pigment epithelium (RPE) or choriod. In some embodiments, the tissue-specific promoter comprises a VMD2 promoter. In some embodiments, the recombinant AAV vector is administered to an eye of the subject using intravitreal or subretinal injection. In some embodiments, administration of the recombinant AAV vector slows, halts, or reverses the loss of retinal ganglion cells (RGC) in the subject; slows, halts, or reverses the loss of Bm3+ retinal ganglion cells (RGC) in the subject; slows, halts, or reverses intraretinal axon degeneration in the subject; slows, halts, or reverses damage to optic nerves of the subject; and/or slows, halts, or reverses the progression of glaucoma in the subject. In some embodiments, administration of the recombinant AAV vector is followed by reattachment of the retina in the subject. In some embodiments, the recombinant AAV vector comprises a nucleotide sequence that encodes a fusion protein fused to a CD5 or CR2 signal peptide. In some embodiments, the recombinant AAV vector comprises one or more sequences from FIG. 22A or FIG. 22B or a human analogue or homologue thereof.

Yet other aspects of the disclosure relate to a recombinant adeno-associated virus (AAV) vector operably encoding a complement receptor 2 (CR2)-complement receptor 1 (CR1) fusion protein; wherein said CR2-CR1 fusion protein comprises a CR2 portion comprising a CR2 or a fragment thereof, and a CR1 portion comprising a CR1 or a fragment thereof; wherein the CR2 portion of the CR2-CR1 fusion protein is capable of binding to a CR2 ligand; and wherein the CR1 portion of the CR2-CR1 fusion protein is capable of inhibiting complement activation. In some embodiments, the AAV vector comprises a recombinant AAV2 vector or a recombinant AAV5 vector. In some embodiments, the AAV vector comprises a chicken β-actin (CBA) promoter or a VMD2 promoter. In some embodiments, the AAV vector comprises one or more sequences from FIG. 22A or FIG. 22B or a human analogue or homologue thereof. In some embodiments, the disclosure provides a pharmaceutical composition comprising the AAV vector

It is to be understood that one, some, or all of the properties of the various embodiments described herein may be combined to form other embodiments of the present disclosure.

### BRIEF DESCRIPTION OF THE FIGURES

FIG. 1 provides schematic diagrams of a GR2-FH expression vector and CR2-FH fusion proteins. The letter “k” refers to sequence, “5” refers to CD5 signal peptide, the letter “l” refers to an optional linker, and the letter “s” refers to stop codon and polyA signal.

FIG. 2 provides the amino acid sequence of human complement receptor 2 (CR2) (SEQ ID NO: 1) and the amino acid sequence of human factor H (FH) (SEQ ID NO: 2).

FIG. 3 provides the amino acid sequence of on exemplary human CR2-FH fusion protein (SEQ ID NO: 3) and an exemplary polynucleotide sequence encoding a human CR2-FH fusion protein (SEQ ID NO: 4).

FIGS. 4-6 provide exemplary amino acid sequences of CR2-FH molecules described herein (SEQ ID NOs: 5-10). “nnn” represents an optional linker.

FIG. 7 provides exemplary amino acid sequences of signaling peptides described herein (SEQ ID NOs: 11, 13, and 25) and exemplary polynucleotide sequences encoding the signaling peptides (SEQ ID NOs: 12, 14, and 26).

FIG. 8 provide an amino acid sequence of mouse CR2 (SEQ ID NO: 15) and amino acid sequence of mouse FH (SEQ ID NO: 16).

FIG. 9 provides an amino acid sequence of an exemplary mouse CR2-FH fusion protein (SEQ ID NO: 17) and an exemplary polynucleotide sequence that encodes a mouse CR2-FH plus the signal peptide (SEQ ID NO: 18).

FIG. 10 provides an exemplary DNA sequence of CR2NLFHFH, a mouse CR2-FH fusion protein containing a CR2 portion and two FH portions without a linker sequence (SEQ ID NO: 19).

FIG. 11 provides an exemplary DNA sequence of CR2LFHFH, a mouse CR2-FH fusion protein containing a CR2 portion linked to two FH portions via a linker sequence (SEQ ID NO: 20).

FIG. 12 provides amino acid sequence of an exemplary human CR2-FH fusion protein (designated as human CR2-fH or CR2fH) (SEQ ID NO: 21) and an exemplary polynucleotide sequence mat encodes a human CR2-fH plus the signal peptide (SEQ ID NO: 22). The first nine nucleotides of SEQ ID NO: 22 correspond to a 5′ untranslated region. The ATG start site begins at nucleotide 10. The first 17 residues encoded by nucleotides 10-60 correspond to the CR2 signal peptide (SEQ ID NO: 26). The sequence encoding the signal peptide is underlined.

FIG. 13 provides an exemplary amino acid sequence of a human CR2-FH fusion protein containing two FH portions (designated as human CR2-FH2 or human CR2fH2) (SEQ ID NO: 23) nod an exemplary polynucleotide sequence that encodes a human CR2-FH2 plus the CR2 signal peptide (SEQ ID NO: 24) Sequence encoding the signal peptide is underlined.

FIG. 14 shows nerve damage level after administration of gene therapy vector AAV2-CR2-Crry and controls in treated and untreated DBA/2J mice.

FIG. 15 shows Brn3+ cell density per retina after administration of gene therapy vector AAV2-CR2-Crry and controls in treated and untreated DBA/2J mice.

FIG. 16A through FIG. 16F shows AAV5-mediated expression of CR2-fH in the mouse RPE. RPE/choroid sections were collected on day 6 post laser-induced photocoagulation, and stained with CR2 antibody before flattened and mounted to glass coverslips for fluorescence microscopy. Mice injected with AAV5-VMD2-mCherry demonstrated red autofluorescence in RPE flatmounts (FIG. 16A), whereas mice injected with AAV5-VMD2-CR2-fH produced green CR2-immunofluorecence (FIG. 16D). As negative controls, mice injected with AAV-mCherry did not did not fluoresce when stained with CR2 (FIG. 16B), and mice injected with AAV5-VMD2-CR2-fH did not demonstrate red auto fluorescence (FIG. 16C). (FIG. 16E) Using dotblot analysis staining for anti-CR2, CR2-fH was detected in mice injected with AAV5-VMD2-CR2-fH in both the RPE/choroid and to a lesser extent in the retina (FIG. 16F). Bioavailability of CR2-fH in RPE/choroid was compared between intravitreal injection of CR2-fH and subretinal injection of AAV5-VMD2-CR2-fH demonstrated comparable results.

FIG. 17A through FIG. 17D shows visual analysis following subretinal AAV5-VMD2-CR2-fH injection. Optomolor response was recorded prior to injection and one month following. By measuring the spacial frequency threshold at a constant speed (12 deg/sec) and contrast (100%), visual acuity was measured. No significant change to visual acuity to mice injected with either AAV-VMD2-mCherry or AAV5-VMD2-CR2-fH was observed (FIG. 17A) (n=14-17 mice per condition). In order to measure contrast sensitivity, the reciprocal of the contrast threshold at a fixed spacial frequency (0.131 cyc/deg) and speed (12 deg/sec) was calculated (n=12-18 mice per condition). As with visual acuity, no significant difference was measured following subretinal injection in either the AAV-VMD2-mCherry or AAV5-VMD2-CR2-fH injected mice (FIG. 17B). Attenuated c-wave amplitudes using full field electroretinography were observed one month following subretinal injection (***P≤0.001), however no gene specific effects from AAV-VMD2-mCherry or AAV5-VMD2-CR2-fH were observed (h=12-14 mice per condition; P=0.65) (FIG. 17D). Focal ERG response was recorded at 3 different flash strengths for a and b waves (FIG. 17D). ERG analysis for areas proximal to the injection (injury) demonstrate an ˜80% reduction in ERG response as opposed to areas distal (non-injury) to the injection site which showed a 20% reduction when compared to non-injected controls (n=10 animals per group. Data are expressed as mean±SEM.

FIG. 18A through FIG. 18G shows RPE cell morphology following AAV5-VMD2-CR2-fH subretinal injection. RPE morphology was determined using the cell junction marker ZO-1. RPE cells surrounding the injection site in both AAV5-VMD2-mCherry (FIG. 18A) and AAV5-VMD2-CR2-fH (FIG. 18B) were unhealthy when compared to RPE cells in the areas outside of the injection (FIG. 18C. AAV5-VMD2-mCherry: FIG. 18D. AAV5-VMD2-CR2-fH). These results were quantified using CellProfiler software to measure RPE health based on normal hexagonal shape (FIG. 18E; n=4 animals per group). A reduced form factor for RPE was observed inside the injection area for both AAV5-VMD2-mCherry and AAV5-VMD2-CR2-fH injected mice although no significant difference was measured. Eccentricity measurement was found to be increased within the injection site for both injection treatments, but again no significance was observed. (FIG. 18F, FIG. 18G) Further analysis of the retina by OCT indicated no distinguishable difference for RPE, outer segments (OS), inner segments (IS) outer nuclear layer (ONL), inner nuclear layer (INL), or whole retina (WR) (n=4-6 mice per condition). Data are expressed as mean±SEM.

FIG. 19 shows subretinal AAV5 treatment does not result in systemic response. We examined whether mice treated with either AAV5-VMD2-mCherry or AAV5-VMD2-CR2-fH developed CR2-fH antibodies. One month following subretinal injection, neither IgG nor IgM antibodies recognizing CR2-fH could be identified in mouse serum using Western blot analysis.

FIG. 20A through FIG. 20C shows subretinal injection with AAV5-VMD2-CR2-fH attenuates CNV. One month following subretinal injection of AAV5-VMD2-mCherry or AAV5-VMD2-CR2-fH, laser-induced CNV was performed and analyzed 5 days following with OCT. A significant decrease in lesion size in mice injected with AAV5-VMD2-CR2-fH was observed (FIG. 20B, FIG. 20C), when compared to control AAV5-VMD2-mCherry (FIG. 20A, FIG. 20C) mice. Data shown are average values (±SEM) per lesion (n=7-8 animals per condition; p≤0.05). Scale bar; 100 pixels.

FIG. 21A through FIG. 21B shows gene expression changes in ocular tissues following subretinal injection of AAV-VMD2-CR2-fH in the presence and absence of CNV. ELISA analysis for RPE/choroid fractions demonstrated a ˜4 fold increase in C3a following laser-induced CNV (FIG. 21A). This effect was completely blocked in the mice treated with AAV5-VMD2-CR2-fH. Quantitative RT-PCR on cDNA generated from RPE/choroid fraction and retina was measured in mice injected with either AAV5-VMD2-mCherry or AAV5-VMD2-CR2-fH in the absence or presence of laser-induced CNV. As expected, C3 and VEGF expression (FIG. 21B) was increased following CNV in uninjected mice and AAV5-VMD2-mCherry injected mice. This effect was blocked in AAV5-VMD2-CR2-fH injected mice. No significant change in RPE-65 was observed in any of the tested condition indicating RPE health was still maintained. Data are expressed as mean±SD (n=3 animals per condition performed in triplicate; p≤0.05).

FIG. 22A through FIG. 22B shows the nucleotide and amino acid sequences for mCR2Crry fusion protein. SEQ ID NO: 37 is a DNA sequence coding a signal peptide, MPMGSLQPLATLYLLGMLVASVLA (SEQ ID NO: 38). SEQ ID NO: 39 encodes the mCR2 portion of the fusion protein: SEQ ID MO: 40 encodes a linker region; and SEQ ID NO: 41 encodes the Crry portion of the fusion protein (FIG. 22A). The amino acid sequence of the mCR2Crry fusion protein (SEQ ID NO: 42) is also shown.

FIG. 23A and FIG. 23B, is a series of images showing subretinal injection with AAV5-VMD2-CR2-fH attenuates Smoke-induced vision loss. Optomotor responses were analyzed in mice treated with cither AAV-VMD2-mCherry or AAV-VMD2-CR2-fH prior to exposure to 6 months of cigarette smoke or room air. (FIG. 23A) Visual acuity was measured by identifying the spatial frequency threshold at a constant speed (12 deg/sec) and contrast (100%). Spatial frequency thresholds were net affected by smoking or gene therapy treatment. (FIG. 23B) Contrast sensitivity was measured by taking the reciprocal of the contrast threshold at a fixed spatial frequency (0.131 cyc/deg) and speed (12 deg/sec). We previously determined that this spatial frequency falls within the range of maximal contrast sensitivity for 9-month-old WT mice (data net shown). Mice injected with the mCherry (control) vector after CE showed a significant reduction in contrast sensitivity (average contrast sensitivity measured over the 6 month time period) compared to room air controls, while AP-inhibited mice (CR2-fH) were significantly projected. Data are expressed as mean ±SEM (n=6-9 per condition). When compared to historic controls mCherry and CR2-fH injected, room-air raised animals did not differ in contrast sensitivity from untransfected mice (untransfected versus mCherry, P=0.37; untransfected versus CR2-fH, p=0.1); and mCherry smoke-exposed animals did not differ from untransfected smoke-exposed mice (P=0.39).

FIG. 24A and FIG. 24B, is a series of images showing optical coherence tomography analysis obtained from mCherry and CR2-fH injected mice exposed to 6 months of cigarette smoke or room air. (FIG. 24A) Posterior poles from room air (lop row) and smoke-exposed (bottom row) mice injected with mCherry (left column) and CCR2-fH (left column) were analyzed in vivo using OCT. OCT measurements were taken ˜0.35 mm from the optic nerve head in the nasal quadrant (see caliper). (FIG. 24B) There is obvious and significant thinning of the whole retina that could not be attributed to any one layer (data not shown) in smoke-exposed mice that could not be corrected for using CR2-fH.

FIG. 25 is an image showing electron micrographs of the RPE/BrM/choriocapillaris complex (RPE/BrM/CC) obtained from mCherry and CR2-fH injected mice exposed to 6 months of cigarette smoke or room air were compared. In animals raised in room air, and injected with mCherry or CR2-fH, the BrM exhibits an organized pentalaminar structure and the RPE exhibits normal basolaminar infolding. Mitochondria are located towards the basal side of the RPE and have an oblong shape. Mice exposed to smoke differ in their response depending on their treatment. mCherry treated mice exhibit pathological changes. BrM is disorganized, losing its pentalaminar structure, and deposits are present within the outer collagenous layer of BrM. Mitochondria are smaller and rounder with with degraded outer membranes and disorganized cristae. In contrast, retinas of animals treated with CR2-fH have a more healthy appearance. BrM has a normal thickness and mitochondria are larger and oblong.

FIG. 26A and FIG. 26B, is a set of morphometric analyses based on images of electron micrographs of the RPE/BrM/choriocapillaris complex (RPE/BrM/CC) obtained from mCherry and CR2-fH injected mice exposed to 6 months of cigarette smoke or room air were analyzed morphometrically. (FIG. 26A) For each animal, a total length of 23 μm of Bruch's membrane was analyzed and thickness measurements obtained. A typical BrM of a young animal is 10 μm thick (data not shown); anything above 10 μm is defined as altered due to aging or disease. BrM health did not differ between mCherry and CR2-fH injected, room-air raised animals (P=0.62), whereas health was significantly impaired in mCherry injected, smoke-exposed mice (mCherry room air versus smoke, P=0.03), whereas health was not impaired in CR2-fH injected, smoke-exposed mice (CR2-fH room air versus smoke, P=0.41). When compared to historic controls mCherry and CR2-fH injected, room-air raised animals did not differ in BrM health from untransfected mice (untransfected versus mCherry, P=0.97; untransfected versus CR2-fH, p=0.d6); and mCherry smoke-exposed animals did not differ from untransfected smoke-exposed mice (P=0.18). (FIG. 26B) For each animal, the volume of basal laminar (BL) infoldings was determined by dividing the area occupied by individual infoldings, by the total BL area for each of the two RPE cells. The BL area was isolated for each RPE cell in Photoshop®. Images were binarized, processed using a median filter (10 px radius) to reduce background noise, and analyzed for area measurements. There was a small but insignificant trend that the volume increases as the percent thickened BrM increases. However, as the subretinal injections altered the BL morphology (uninjected air versus mCherry air P<0.05; uninjected air versus CR2-fH air P<0.03), a treatment effect after smoke exposure could not be identified. This is consistent with our previous finding that the basolaminar infolding area is not susceptible to complement status. CFB^{-/-} mice lost as much basolaminar infolding area in response to smoke as did wildtype animals.

FIG. 27A and FIG. 27B is a set of morphometric analyses based on images of electron micrographs of the RPE obtained from mCherry and CR2-fH injected mice exposed to 6 months of cigarette smoke or room air were analyzed morphometrically for mitochondrial area and shape. (FIG. 27A) The area of the mitochondria in the area of thickened membranes was determined by masking each mitochondria and subtracting that from the overall area of the RPE. As the number of the mitochondria was unchanged by smoke (P=0.12−0.61; data not shown), smoke reduced the size of the mitochondria (fragmentation) in mCherry, but not in CR2-fH-treated animals. (FIG. 27B) Each mitochondria per condition was assessed based on shape (Image J, circularity index) and assigned to 10 bins. The peak of the distribution for mCherry and CR2-fH air is 0.8, as is CR2-fH smoke, whereas the curve for mCherry smoke is significantly shifted to the right. Specifically, a Related-Samples Wilcoxon Signed Rank Test revealed significant differences between air and smoke for mCherry (P<0.001) but not for CR2-fH (P=0.13); as well as within the smoked group for mCherry versus CR2-fH (P<0.001).

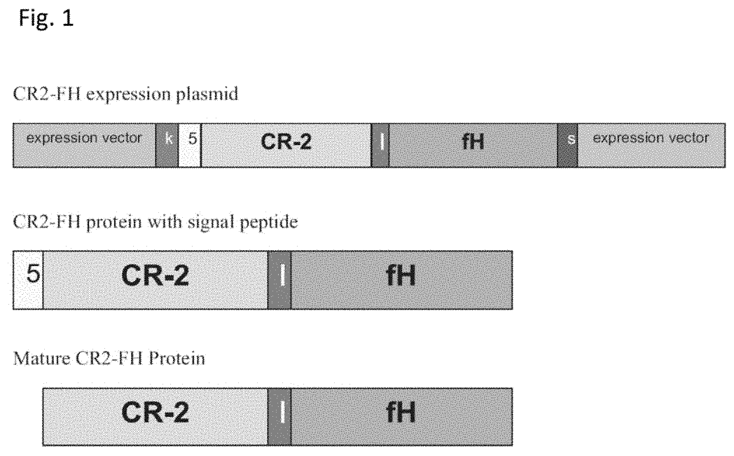

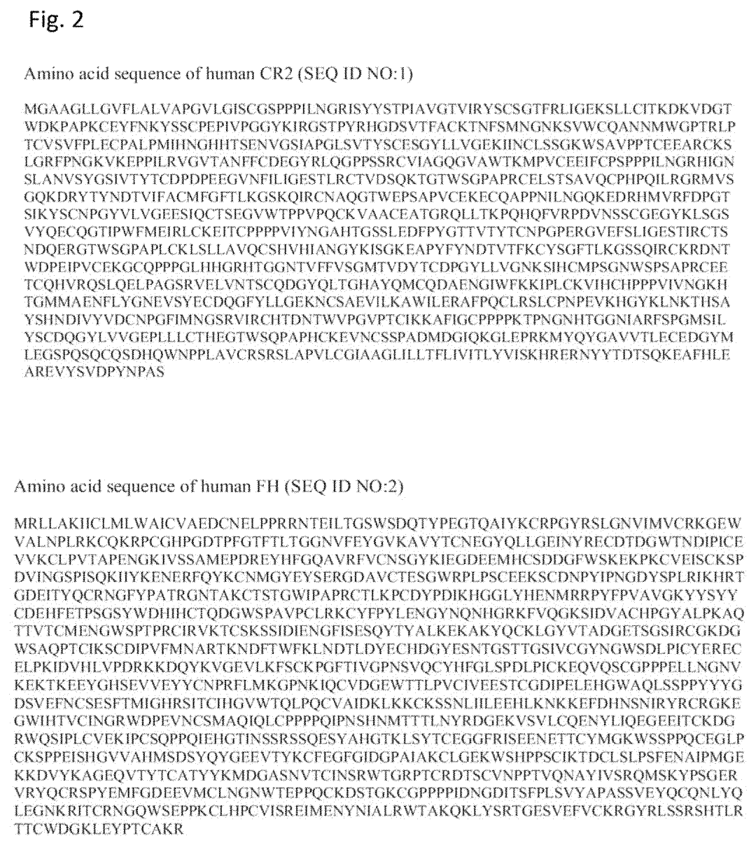

### DETAILED DESCRIPTION

The disclosure provides a method for treating eye disease in a subject afflicted with, or at risk for, eye disease. The subject can be a human subject or non-human subject (e.g., a veterinary subject such as a domestic dog or cat). In some embodiments, the eye disease comprises a progressive eye disease such as glaucoma or macular degeneration (MD), including age-related macular degeneration (AMD). In some embodiments, the eye disease is one in which the alternative complement pathway is implicated. The treatment method involves what is commonly known as “gene therapy,” in which a recombinant vector, such as a recombinant adeno-associated virus (AAV) vector, which operably encodes a CR2-FH fusion protein (including, without limitation, any of the CR2-FH fusion protein embodiments described herein, as well as CR2-FH variants as described herein) is administered to a subject afflicted with, or at risk for, an eye disease. In some embodiments, the AAV vector is introduced directly into eye tissue of the sublet, e.g., via intraocular administration. Introduction of the recombinant AAV vector into the subject permits in vivo expression of the CR2-FH fusion protein to achieve a therapeutic effect.

The disclosure thus also provides a recombinant vector, such as a recombinant AAV vector, which operably encodes a CR2-FH fusion protein. The CR2-FH fusion protein can include, without limitation, any of the CR2-FH fusion protein embodiments described herein, as well as CR2-FH variants as described herein. The disclosure also provides compositions, such as pharmaceutical compositions, comprising a recombinant vector, such as an AAV vector, that operably encodes a CR2-FH fusion protein which likewise includes, without limitation, any of the CR2-FH fusion protein embodiments described herein, as well as CR2-FH variants as described herein. The pharmaceutical composition optionally includes a pharmaceutically acceptable carrier.

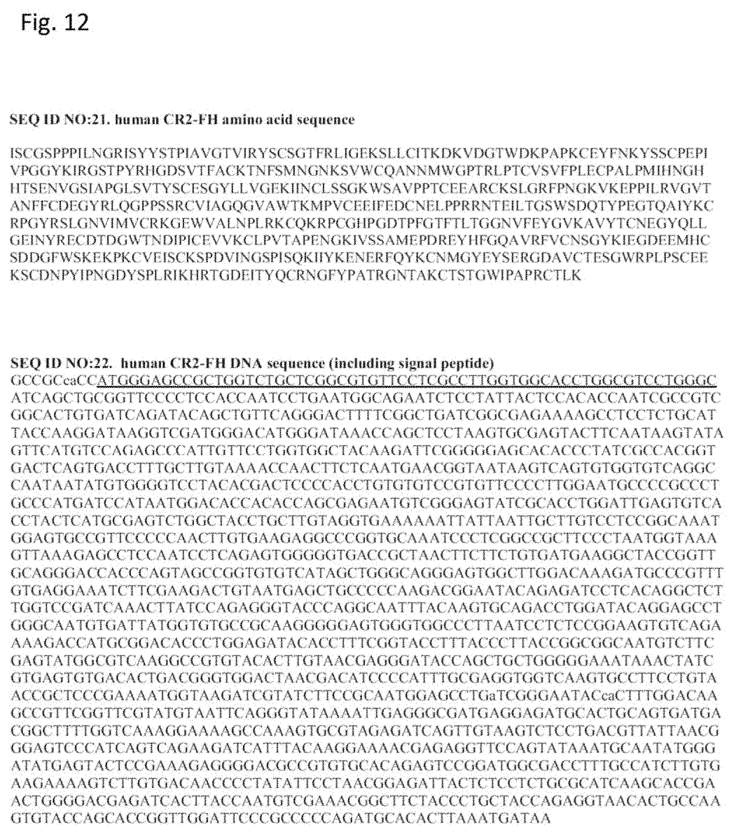

The CR2-FH fusion protein encoded by the recombinant vector comprises: a) a CR2 portion comprising a CR2 or a fragment thereof, and b) a FH portion comprising a FH or a fragment thereof. The CR2 portion is capable of binding to one or more natural ligands of CR2 and is thus responsible for targeted delivery of the molecule to the sites of complement activation, and the FH portion is responsible for specifically inhibiting complement activation of the alternative pathway. An illustrative CR2-FH fusion protein containing the first four N-terminal SCR domains of the CR2 protein and the first five N-terminal SCR domains the factor H protein (SEQ ID NO: 21. FIG. 12), has both targeting activity and complement inhibitory activity in vitro (see U.S. Pat. No. 7,759,304). This molecule is significantly more effective than a factor H molecule lacking the CR2 portion.

As used herein, the singular form “a”, “an”, and “the” includes plural references unless indicated otherwise. For example, “a” FH portion includes one or more FH portions.

Reference to “about” a value or parameter herein includes (and describes) embodiments that are directed to that value or parameter per se. For example, description referring to “about X” includes description of “X”.

It is understood (hat aspects and embodiments of the disclosure described herein include “consisting” and/or “consisting essentially of” aspects and embodiments.

### CR2-FH Fusion Protein

Representative CR2-FH fusion proteins are described herein; sec also U.S. Pat. No. 7,759, 304 for additional embodiments of the CR2-FH fusion protein, as well as further description of the CR2-FH fusion protein and related methods, including synthetic methods and activity assays.

“CR2-FH molecule” and “CR2-FH fusion protein” are used interchangeably herein, and refer to a non-naturally occurring molecule comprising a CR2 or a fragment thereof (the “CR2 portion”) and a FH or a fragment thereof (the “FH portion”). The CR2-FH fusion proteins can be assayed for biological activity using in vitro or in vivo assays known to the art, for example, as set forth in U.S. Pat. No. 7,759,304.

The CR2-FH fusion protein described herein thus generally has the dual functions of binding to a CR2 ligand and inhibiting complement activation of tire alternative pathway. “CR2 ligand”)refers to any molecule that binds to a naturally occurring CR2 protein, which include, but are not limited to, C3d, iC3b, C3dg, C3d, and cell-bound fragments of C3b that bind to the two N-terminal SCR domains of CR2. The CR2-FH fusion protein may, for example, bind to a CR2 ligand with a binding affinity that is about any of 10%, 20%, 30%, 40%, 50%, 60%, 70%, 80%, 90%, or 100% of the CR2 protein. Binding affinity can be determined by any method known in the art, including for example, surface plasmon resonance, calorimetry titration, ELISA, and flow cytometry. In some embodiments, the CR2-FH fusion protein has one or more of the following properties of CR2: (1) binding to C3d, (2) binding to iC3b, (3) binding to C3dg, (4) binding to C3d, and (5) binding to cell-bound fragments) of C3b that bind to the two N-terminal SCR domains of CR2.

The CR2-FH fusion protein described herein is generally capable of inhibiting complement activation of the alternative pathway. The CR2-FH fusion protein may be a more potent complement inhibitor than the naturally occurring FH protein. For example, in some embodiments, the CR2-FH fusion protein has a complement inhibitory activity that is about any of 1.5, 2, 2.5, 3, 3.5, 4, 5, 6, 7, 8, 9, 10, 12, 14, 16, 18, 20, 25, 30, 40, or more fold of that of the FH protein. In some embodiments, the CR2-FH fusion protein has an EC50 of less than about any of 100 nM, 90 nM, 80 nM, 70 nM, 60 nM, 50 nM, 40 nM, 30 nM, 20 nM, or 10 nM. In some embodiments, the CR2-FH fusion protein has an EC50 of about 5-60 nM, including for example any of 8-50 nM, 8-20 nM, 10-10 nM, and 20-30 nM. In some embodiments, the CR2-FH fusion protein has complement inhibitory activity that is about any of 50%, 60%, 70%, 80%, 90%, or 100% of that of the FH protein.

Complement inhibition can be evaluated based on any methods known in the art, including for example, in vitro zymosan assays, assays for lysis of erythrocytes, immune complex activation assays, and mannan activation assays. In some embodiments, the CR2-FH has one or more of the following properties of FH: (1) binding to C-reactive protein (CRP), (2) binding to C3b, (3) binding to heparin, (4) binding to sialic acid, (5) binding to endothelial cell surfaces, (6) binding to cellular integrin receptor, (7) binding to pathogens, (8) C3b co-factor activity, (9) C3b decay-acceleration activity, and (10) inhibiting the alternative complement pathway.

In some embodiments, the CR2 portion and the FH portion of the CR2-FH fusion protein are directly fused to each other. In some embodiments, the CR2 portion and the FH portion are linked by an amino acid linker sequence. Examples of linker sequences are known in the art and include, for example, (Gly₄Ser), (Gly₄Ser)₂, (Gly₄Ser)₃, (Gly₃Ser)₄, (SerGly₄), (SerGly₄)₂, (SerGly₄)₃, and (SerGly₄)₄. Linking sequences can also comprise “natural” linking sequences found between different domains of complement factors. For example, VSVFPLE (SEQ ID NO: 27), the linking sequence between the first two N-terminal short consensus repeat (SCR) domains of human CR2, can be used. In some embodiments, the linking sequence between the fourth and the fifth N-terminal short consensus repeat domains of human CR2 (EEIF; SEQ ID NO: 28) is used. The order of CR2 portion and FH portion in the fusion protein can vary. For example, in some embodiments, the C-terminus of the CR2 portion is fused (directly or indirectly) to the N-terminus of the FH portion of the fusion protein. In some embodiments, the N-terminus of the CR2 portion is fused (directly or indirectly) to the C-terminus of the FH portion of the fusion protein.

In some embodiments, the CR2-FH fusion protein has an amino acid sequence of any of SEQ ID NO: 3, SEQ ID NO: 21, and SEQ ID NO: 23. In some embodiments, the CR2-FH fusion protein has an amino acid sequence that is at least about 50%, 60%, 70%, 80%, 90%, 91%, 92%, 93%, 94%, 95%, 96%, 97%, 98%, or 99% identical to that of any or SEQ ID NO:3, SEQ ID NO:21, or SEQ ID NO:23. In some embodiments, the CR2-FH fusion protein comprises at least about 400, 450, 500, 550, or more contiguous amino acids of any of SEQ ID NO: 3, SEQ ID NO: 21, and SEQ ID NO: 23.

In some embodiments, the CR2-FH fusion protein has an amino acid sequence of any or SEQ ID NOs:5-10. In some embodiments, the CR2-FH fusion protein has an amino acid sequence that is at least about 50%, 60%, 70%, 80%, 90%, 91%, 92%, 93%, 94%, 95%, 96%, 97%, 98%, or 99% identical to that of any of SEQ ID NOs: 5-10. In some embodiments, the CR2-FH fusion protein comprises at least about 400, 450, 500, 550, or more contiguous amino acids any of SEQ ID NOs: 5-10.

In some embodiments, the CR2-FH fusion protein is encoded by a polynucleotide having nucleic acid sequence of any of SEQ ID NO:4, SEQ ID NO:22, and SEQ ID NO: 24. In some embodiments, the CR2-FH fusion protein is encoded by a polynucleotide having a nucleic acid sequence that is at least about 50%, 60%, 70%, 80%, 90%, 91%, 92%, 93%, 94%, 95%, 96%, 97%, 98%, or 99% identical to that of any of SEQ ID NO: 4, SEQ ID NO: 22, and SEQ ID NO: 24.

In some embodiments, the CR2-FH fusion protein comprises two or more (same or different) CR2 portions described herein. In some embodiments, the CR2-FH fusion protein comprises two or more (same or different) FH portions described herein. These two or more CR2 (or FH) portions may be tandemly linked (such as fused) to each other. In some embodiments, the CR2-FH fusion protein comprises a CR2 portion and two or more (such as three, four, five, or more) FH portions. In some embodiments, the CR2-FH fusion protein comprises a FH portion and two or more (such as three, four, five, or more) CR2 portions. In some embodiments, the CR2-FH fusion protein comprises two or more CR2 portions and two or more FH portions.

The CR2 portion and the FH portion in the fusion protein can be from the same species (such as human or mouse), or from different species.

### CR2 Portion

The CR2 portion described herein comprises a complement receptor 2 (also known as complement receptor type 2 or CR2) or a fragment thereof.

CR2 is a transmembrane protein expressed predominantly on mature B cells and follicular dendritic cells. CR2 is a member of the C3 binding protein family. Natural ligands for CR2 include, for example, iC3b, C3dg, and C3d, and cell-bound breakdown fragments of C3b that bind to the two N-terminal SCR domains of CR2. Cleavage of C3 results initially in the generation of C3b and the covalent attachment of this C3b to the activating cell surface. The C3b fragment is involved in the generation of enzymatic complexes that amplify the complement cascade. On a cell surface, C3b is rapidly converted to inactive iC3b, particularly when deposited on a host surface containing regulators of complement activation (i.e., most host tissue). Even in absence of membrane bound complement regulators, substantial levels of iC3b are formed. iC3b is subsequently digested to the membrane bound fragments C3dg and then C3d by serum proteases, but this process is relatively slow. Thus, the C3 ligands for CR2 are relatively long lived once they are generated and will be present in high concentrations at sites of complement activation. CR2 therefore can serve as a potent targeting vehicle for bringing molecules to the site of complement activation.

CR2 contains an extracellular portion having 15 or 16 repeating units known as short consensus repeats (SCR domains). The SCR domains have a typical framework of highly conserved residues including four cysteines, two prolines, one tryptophan and several other partially conserved glycines and hydrophobic residues. SEQ ID NO: 1 represents the full-length human CR2 protein sequence. Amino acids 1-20 comprise the leader peptide, amino acids 23-82 comprise SCR1, amino acids 91-146 comprise SCR2, amino acids 154-210 comprise SCR3, and amino acids 215-271 comprise SCR4. The active site (C3d binding site) is located in SCR 1-2 (the first two N-terminal SCR domains). These SCR domains are separated by short sequences of variable length that serve as spacers. The full-length mouse CR2 protein sequence is represented herein by SEQ ID NO: 15. The SCR1 and SCR2 domains of the mouse CR2 protein are located with the mouse CR2 amino sequence at positions 14-73 of SEQ ID NO: 15 (SCR1) and positions 82-138 of SEQ ID NO: 15(SCR2). Human and mouse CR2 are approximately 66% identical over the full length amino acid sequences represented by SEQ ID NO: 1 and SEQ ID NO: 15, and approximately 61% identical over the SCR1-SCR2 regions of SEQ ID NO: 1 and SEQ ID NO: 15. Both mouse and human CR2 bind to C3 (in the C3d region). It is understood that species and strain variations exist for the disclosed peptides, polypeptides, and proteins, and that the CR2 or a fragment thereof described herein encompasses all species and strain variations.

The CR2 portion disclosed herein refers to a polypeptide that contains some or all of the ligand binding sites of the CR2 protein, and includes, but is not limited to, full-length CR2 proteins (such as human CR2 as shown in SEQ ID NO: 1 or mouse CR2 as shown in SEQ ID NO: 15). soluble CR2 proteins (such as a CR2 fragment comprising the extracellular domain of CR2). other biologically active fragments of CR2, a CR2 fragment comprising SCR1 and SCR2, or any homologue of a naturally occurring CR2 or fragment thereof, as described in detail below. In some embodiments, the CR2 portion has one of the following properties of CR2: (1) binding to C3d, (2) binding to iC3b, (3) binding to C3dg, (4) binding in C3d, and (5) binding in cell-bound fragment(s) of C3b that bind in the two N-terminal SCR domains of CR2.

In some embodiments, the CR2 portion comprises the first two N-terminal SCR domains of CR2. In some embodiments, the CR2 portion comprises the first three N-terminal SCR domains of CR2. In some embodiments, the CR2 portion comprises the first four N-terminal SCR domains of CR2. In some embodiments, the CR2 portion comprises (and in some embodiments consists of or consists essentially of) at least the first two N-terminal SCR domains of CR2, including for example at least any of the first 3, 4, 5, 6, 7, 8, 9, 10, 11, 12, 13, 14, 15, or 16 SCR domains of CR2.

A homologue of a CR2 protein or a fragment thereof includes proteins which differ from a naturally occurring CR2 (or CR2 fragment) in that at least one or a few amino acids have been deleted (e.g., a truncated version of the protein, such as a peptide or fragment), inserted, inverted, substituted and/or derivatized (e.g., by glycosylation, phosphorylation, acetylation, myristoylation, prenylation, palmitation, amidation and/or addition of glycosylphosphatidyl inositol). In some embodiments, a CR2 homologue has an amino acid sequence that is at least about 70% identical to the amino acid sequence of a naturally occurring CR2 (e.g., SEQ ID NO: 1, or SEQ ID NO: 15), for example at least about any of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of a naturally occurring CR2 (e.g., SEQ ID NO: 1, or SEQ ID NO: 15). A CR2 homologue or a fragment thereof preferably retains the ability to bind to a naturally occurring ligand of CR2 (e.g.,. C3d or other C3 fragments with CR2-binding ability). For example, the CR2 homologue (or fragment thereof) may have a binding affinity for C3d that is at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of that or CR2 (or a fragment thereof).

In some embodiments, the CR2 portion comprises at least the first two N-terminal SCR domains of a human CR2, such as a CR2 portion having an amino acid sequence containing at least amino acids 23 through 146 of the human CR2 (SEQ ID NO: 1). In some embodiments, the CR2 portion comprises at least the first two SCR domains of human CR2 having an amino acid sequence that is at least about any of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to amino acids 23 through 146 of the human CR2 (SEQ ID NO: 1).

In some embodiment, the CR2 portion comprises at least the first four N-terminal SCR domains of a human CR2, such as a CR2 portion having an amino acid sequence containing at least amino acids 23 through 271 or the human CR2 (SEQ ID NO: 1). In some embodiments, the CR2 portion comprises at least the first four SCR domains of human CR2 having an amino acid sequence that is at least about any of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to amino acids 23 through 271 of the human CR2 (SEQ ID NO: 1).

An amino acid sequence that is at least about, for example, 95% identical to a reference sequence (such as SEQ ID NO: 1) is intended that the amino acid sequence is identical to the reference sequence except that the amino acid sequence may include up to five point alterations per each 100 amino acids of the reference sequence. These up to five point alterations may be deletions, substitutions, additions, and may occur anywhere in the sequence, interspersed either individually among amino acids in the reference sequence or in one or more continuous groups within the reference sequence.

In some embodiments, the CR2 portion comprises part or all of the ligand binding sites of the CR2 protein. In some embodiments, the CR2 portion further comprises sequences required to maintain the three dimensional structure of the binding site. Ligand binding sites of CR2 can be readily determined based on the crystal structures of CR2, such as the human and mouse CR2 crystal structures disclosed in U.S. Patent Application Publication No. 2004/0005538. For example, in some embodiments, the CR2 portion comprises the B strand and B-C loop of SCR2 of CR2. In some embodiments, the CR2 portion comprises a site on strand B and the B-C loop of CR2 SCR comprising the segment G98-G99-Y100-K101-I102-R103-G104-S105-T106-P107-Y108 with respect to SEQ ID NO: 1. In some embodiments, the CR2 portion comprises a site on the B strand of CR2 SCR2 comprising position K119 with respect to SEQ ID NO: 1. In some embodiments, the CR2 portion comprises a segment comprising V149-F150-P151 -L152, with respect to SEQ ID NO: 1. In some embodiments, the CR2 portion comprises a segment of CR2 SCR2 comprising T120-N121-F122. In some embodiments, the CR2-FH fusion protein has two or more of these sites. For example, in some embodiments, the CR2 portion comprises a portion comprising G98-G99-Y100-K101-I102-R103-G104-S105-T106-P107-Y108 and K119 with respect to SEQ ID NO: 1. Other combinations of these sites are also contemplated.

### FH Portion

The FH portion of the CR2-FH fusion protein described herein comprises a Factor H (FH) or a fragment thereof.

Complement factor H (FH) is a single polypeptide chain plasma glycoprotein. The protein is composed of 20 repetitive SCR domains of approximately 60 amino acids, arranged in a continuous fashion like a string of 20 beads. Factor H binds to C3b, accelerates the decay of the alternative pathway C3-convertase (C3Bb), and acts as a cofactor for the proteolytic inactivation of C3b. In the presence of factor H, C3b proteolysis results in the cleavage of C3b. Factor II has at least three distinct binding domains for C3b, which are located within SCR 1-4, SCR 5-8, and SCR 19-20. Each site of factor H binds to a distinct region within the C3b protein: the N-terminal sites bind to native C3b; the second site, located in the middle region of factor H, binds to the C3c fragment and the sited located within SCR 19 and 20 binds to the C3d region. In addition, factor H also contains binding sites for heparin and well as complexes of other physiological polyanions, which are located within SCR 7, SCR 5-12, and SCR20 of factor H and overlap with that of the C3b binding site. Structural and functional analyses have shown that the domains for the complement inhibitory and/or regulatory activity of FH (e.g., decay-acceleration and cofactor) are located within the first four N-terminal SCR domains.

SEQ ID NO: 2 represents the full-length human FH protein sequence. Amino acids 1-18 correspond to the leader peptide, amino acids 21-80 correspond to SCR1, amino acids 85-141 correspond to SCR2, amino acids 146-205 correspond to SCR3, amino acids 210-262 correspond to SCR4, amino acids 267-320 correspond to SCR5. The full-length mouse FH protein sequence is represented herein by SEQ ID NO: 16. The SCR1 and SCR2 domains of the mouse FH protein are located with the mouse FH amino sequence at positions 21-27 of SEQ ID NO: 16 (SCR1) and positions 82-138 of SEQ ID NO: 16(SCR2). Human and mouse FH are approximately 61% identical over the full length amino acid sequences represented by SEQ ID NO: 2 and SEQ ID NO: 16. It is understood that species and strain variations exist for the disclosed peptides, polypeptides, and proteins, and that the FH or a fragment thereof encompasses all species ant strain variations.

The FH portion described herein refers to any portion of a FH protein having some or all the complement inhibitory activity of the FH protein, and includes, bill is not limited to, full-length FH proteins, biologically active fragments of FH proteins, a FH fragment comprising SCR1-4, or any homologue of a naturally occurring FH or fragment thereof, as described in detail below. In some embodiments, the FH portion has one or more of the following properties: (1) binding to C-reactive protein (CRP), (2) binding to C3b, (3) binding to heparin, (4) binding to sialic acid, (5) binding to endothelial cell surfaces, (6) binding to cellular integrin receptor, (7) binding to pathogens, (8) C3b co-factor activity, (9) C3b decay-acceleration activity, and (10) inhibiting the alternative complement pathway.

In some embodiments, the FH portion comprises the first four N-terminal SCR domains of FH. In some embodiments, the construct comprises the first five N-terminal SCR domains of FH. In some embodiments, the construct comprises the first six N-terminal SCR domains of FH. In some embodiments, the FH portion comprises (and in some embodiments consists of or consisting essentially of) at least the first four N-terminal SCR domains of FH, including for example, at least any of the first 5, 6, 7, 8, 9, 10, 11, 12, 13, 14, or more N-terminal SCR domains of FH.

In some embodiments, the FH is a wildtype FH. In some embodiments, the FH is a protective variant of FH.

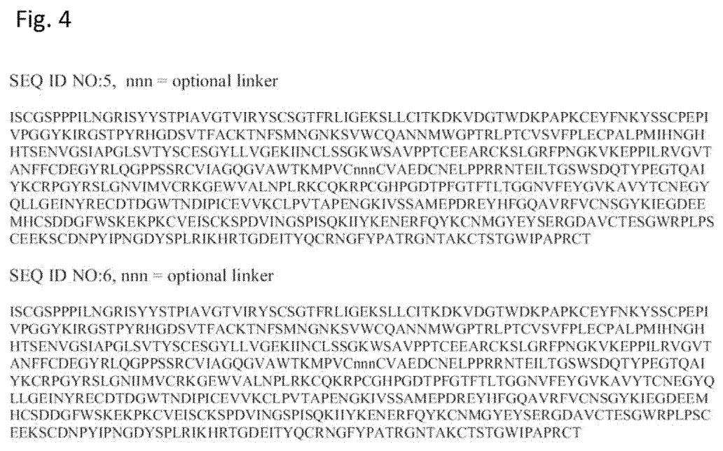

In some embodiments, the FH portion lacks a heparin binding site. This can be achieved, for example, by mutation of the heparin binding site on FH, or by selecting FH fragments that do not contain a heparin binding site. In some embodiments, the FH portion comprises a FH or a fragment thereof having a polymorphism that is protective to age-related macular degeneration. Hageman et al., Proc. Natl. Acad. Sci. USA 102(20):7227. One example of a CR2-FH molecule comprising such a sequence is provided in FIG. 4 (SEQ ID NO: 6).

A homologue of a FH protein or a fragment thereof includes proteins which differ from a naturally occurring FH (or FH fragment) in that at least one or a few, but not limited to one or a few, amino acids have been deleted (e.g., a truncated version of the protein, such as a peptide or fragment), inserted, inverted, substituted and/or derivatized (e.g., by glycosylation, phosphorylation, acetylation, myristoylation, prenylation, palmitation, amidation and/or addition of glycosylphosphatidyl inositol). For example, a FH homologue may have an amino acid sequence that is at least about 70% identical to the amino acid sequence of a naturally occurring FH (e.g., SEQ ID NO: 2, or SEQ ID NO: 16), for example at least about any of 75%, 76%, 77%, 78%, 78%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of a naturally occurring FH (e.g., SEQ ID NO: 2, or SEQ ID NO: 16). In some embodiment, a homologue of FH (or a fragment thereof) retains all the complement inhibition activity of FH (or a fragment thereof). In some embodiments, the homologue of FH (or a fragment thereof) retains at least about 50%, for example, at least about any of 60%, 70%, 80%, 90%, or 95% of the complement inhibition activity of FH (or a fragment thereof).

In some embodiments, the FH portion comprises at least the first four N-terminal SCR domains of a human FH, such as a FH portion having an amino acid sequence containing at least amino acids 21 through 262 of the human FH (SEQ ID NO: 2). In some embodiments, the FH portion comprises at least the first four N-terminal SCR domains of human FH having an amino acid sequence that is at least about any of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to amino acids 21 through 262 of the human FH (SEQ ID NO: 2).

In some embodiments, the FH portion comprises at least the first five N-terminal SCR domains of a human FH, such as a FH portion having an amino acid sequence containing at least amino acids 21 through 320 of the human FH (SEQ ID NO: 2). In some embodiments, the FH portion comprises at least the first five N-terminal SCR domains of human FH having an amino acid sequence that is at least about any of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to amino acids 21 through 320 or the human FH (SEQ ID NO: 2).

In some embodiments, the FH portion comprises a full length or a fragment of factor-H like 1 molecule (FHL-1), a protein encoded by an alternatively spliced transcript of the factor H gene. The mature FHL-1 contains 431 amino acids. The first 427 amino acids organize seven SCR domains and are identical to the N-terminal SCR domains of FH. The remaining four amino acid residues Ser-Phe-Thr-Leu (SFTL) at the C-terminus are specific to FHL-1. FHL-1 has been characterized functionally and shown to have factor H complement regulatory activity. The term “FH portion” also encompasses full length or fragments of factor H related molecules, including, but are not limited to, proteins encoded by the FHR1, FHR2, FHR3, FHR4, FHR5 genes. These factor H related proteins are disclosed, for example, in de Cordoba et al., Molecular Immunology 2004, 41:355-367.

### CR2-FH Fusion Protein Variants

In some embodiments, the recombinant vector operably encodes a variant of a CR2-FH fusion protein. A variant of the CR2-FH fusion protein may be: (i) one in which one or more of the amino acid residues of the CR2 portion and/or the FH portion are substituted with a conserved or non-conserved amino acid residue (preferably a conserved amino acid residue) and such substituted amino acid residue may or may not be one encoded by the genetic code; or (ii) one in which the CR2-FH fusion protein is fused with another compound, such as a compound to increase the half-life of the CR2-FH fusion protein, or (iii) one in which additional amino acids are fused to the CR2-FH fusion protein, such as a leader or secretory sequence, or (iv) one in which the CR2-FH fusion protein is fused with a larger polypeptide, i.e., human albumin, an antibody or Fc, for increased duration of effect. Such variants are deemed to be within the scope of those skilled in the an from the teachings herein.

In some embodiments, the variant of the CR2-FH fusion protein contains conservative amino acid substitutions (defined further below) made at one or more amino acid residues, preferably at one or more amino acid residual that are known to be, or predicted to be, nonessential. A “nonessential” amino acid residue is a residue that can be altered from the wild-type sequence of a protein without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families induce amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Amino acid substitutions in the CR2 or FH portions of the CR2-FH fusion protein can be introduced to improve the functionality of the molecule. For example, amino acid substitutions can be introduced into the CR2 portion of the molecule to increase binding affinity of the CR2 portion to its ligand(s), increase binding specificity of the CR2 portion to its ligand(s), improve targeting of the CR2-FH fusion protein to desired sites, increase dimerization or multimerization of CR2-FH molecules, and improve pharmacokinetics of the CR2-FH fusion protein. Similarly, amino acid substitutions can be introduced into the FH portion of the molecule to increase the functionality of the CR2-FH fusion protein and improve pharmacokinetics of the CR2-FH fusion protein.

In some embodiments, the CR2-FH fusion protein is fused with another polypeptide, such as a polypeptide to increase the half-life reduce potential immunogenicity, and/or to increase the targeting efficiency of the CR2-FH fusion protein. For example, the CR2-FH fusion protein can be fused to a ligand (such as an amino acid sequence) that has the capability to bind or otherwise attach to an endothelial cell of a blood vessel (referred to as “vascular endothelial targeting amino acid ligand”). Exemplary vascular endothelial targeting ligands include, but are not limited to, VEGF, FGF, integrin, fibronectin, 1-CAM, PDGF, or an antibody to a molecule expressed on the surface of a vascular endothelial cell.

For treatment of eye diseases such as AMD that are associated with the occurrence of drusen, the CR2-FH can be conjugated (such as fused) to an antibody that recognizes a neoepitope of the drusen. Other targeting molecules such as small targeting peptide can also be used.

The CR2-FH fusion protein may include the addition of an immunologically active domain, such as an antibody epitope or other tag, to facilitate targeting of the polypeptide. Other amino acid sequences that may be included in the CR2-FH molecule include functional domains, such as active sites from enzymes such as a hydrolase, glycosylation domains, and cellular targeting signals.

Variants of the CR2-FH fusion protein include polypeptides having an amino acid sequence sufficiently similar to the amino acid sequence of the CR2-FH fusion protein. The term “sufficiently similar” means a first amino acid sequence that contains a sufficient or minimum number of identical, or equivalent amino acid residues relative to a second amino acid sequence such that the first and second amino acid sequences have a common structural domain and/or common functional activity. For example, amino acid sequences that contain a common structural domain that is at least about 45%, preferably about 75% through 98%, identical are defined herein as sufficiently similar. Variants include variants of fusion proteins encoded by a polynucleotide that hybridizes to a polynucleotide encoding a CR2-FH fusion protein, or a complement thereof, under stringent conditions. Such variants generally retain the functional activity of the CR2-FH fusion proteins. Libraries of fragments of the polynucleotides can be used to generate a variegated population of fragments for screening and subsequent selection. For example, a library of fragments can be generated by treating a double-stranded PCR fragment of a polynucleotide with a nuclease under conditions wherein nicking occurs only about once per molecule, denaturing the double-stranded DNA, renaturing the DNA to form double-stranded DNA which can include sense/antisense pairs from different nicked products, removing single-stranded portions from reformed duplexes by treatment with S1 nuclease, and ligating resulting fragment library into an expression vector. By this method, one can derive an expression library that encodes N-terminal and internal fragments of various sizes of the CR2-FH fusion proteins.

Variants include fusion proteins that differ in amino acid sequence due to mutagenesis. In addition, bioequivalent analogs of the CR2-FH fusion protein may also be constructed by making various substitutions on residues or sequences in the CR2 portion and/or the FH portion.

In some embodiments, the CR2-FH fusion protein is fused at its N-terminus to a signal peptide. Such signal peptides are useful for the secretion of the CR2-FH fusion protein. Suitable signal peptides include, for example, the signal peptide of the CD5 protein (such as signal peptide of the human CDS protein MPMGSLQPLATLYLLGMLVAS, SEQ ID NO: 11). In some embodiments, the signal peptide of the CR2 protein is used. For example, in some embodiments, the signal peptide of the human CR2 protein (MGAAGLLGVFLALVAPG, SEQ ID NO: 13 or MGAAGLLGVFLALVAPGVLG, SEQ ID NO: 25) is used.

### Recombinant Vectors Encoding CR2-FH Fusion Proteins

The disclosure provides a recombinant vector that operably encodes a CR2-FH fusion protein comprising a CR2 portion comprising a CR2 or a fragment thereof, and a FH portion comprising a FH or a fragment thereof (with or without a linker sequence). The CR2-FH fusion protein encoded by the recombinant victor can include, without limitation, any of the CR2-FH fusion protein embodiments described herein, as well as CR2-FH variants as described herein. The recombinant vector can be delivered to a subject during gene therapy such that it transfects a cell of the subject that is capable of expressing the CR2-FH fusion protein. The CR2-FH fusion protein can thus be provided “in situ” by introduction of an expression vector to the tissue of interest which then expresses the CR2-FH fusion protein.

Suitable expression vectors include viral or non-viral vectors (e.g., plasmids). Viral vectors include retroviruses, herpes viruses, parvoviruses, adenoviruses, and adeno-associated viruses. See, e.g., Ali et al. (1994) Gene Therapy 1:367-384; U.S. Pat. No. 4,797,368, incorporated herein by reference, and U.S. Pat. No. 5,139,941. In some embodiments, gene delivery vectors which direct expression of CR2-FH in the eye are used. Certain vectors for gene delivery to the eye are known in the art, and have been disclosed, for example, in U.S. Pat. No. 6,943,153, and U.S. Patent Application Publication Nos. US2002/0194630, US2003/0129164, and US2006/00627165.

The recombinant vector may include any element to establish a conventional function of an expression vector, for example, promoter, terminator, selection marker, and origin of replication. Mammalian expression vectors may contain non-transcribed elements such as an origin of replication, promoter and enhancer, and 5′ or 3′ untranslated sequences such as ribosome binding sites, a polyadenylation site, acceptor site and splice donor, and transcriptional termination sequences.

In some embodiments the nucleic acid sequence encoding a CR2-FH fusion protein is under the control of a promoter. The recombinant AAV can include any suitable promoter, which is operably linked to a nucleotide sequence encoding the CR2-FH fusion protein. As used herein, the term “operably linked” refers to two or more nucleic acid or amino acid sequence elements dial are physically linked in such a way dial they ate in a functional relationship with each other. For instance, a promoter is operably linked to a coding sequence if the promoter is able to initiate or otherwise control/regulate the transcription and/or expression of a coding sequence, in which case the coding sequence should be understood as being “under the control of” the promoter. Generally, when two nucleic acid sequences are operably linked, they will be in the same orientation and usually also in the same reading frame. They will usually also be essentially contiguous, although this may not be required. A promoter can be constitutive, regulative or inducible. In some embodiments, the vector has a promoter that drives expression in multiple cell types. In some embodiments, the vector has a promoter that drives expression in specific cell types, such as cells of retina, for example, retinal pigment epithelium (RPE) cells or retinal ganglion cells (RGC). Examples of promoters suitable for expression in RPE cells include RPE65 promoter and VMD2 promoter. See, e.g., McClements et al., Transl. Res. 2013, 161(4):10.1016/j.trsl.2012.12.007. Other promoters which may be employed include, but are not limited to, cellular promoters such as eukaryotic cellular promoters including, but not limited to, the histone, pol III, and β-actin promoters, such as chicken β-actin (CBA) promoter and the like, and viral promoters such as retroviral LTR promoter, simian virus 40 (SV40) promoter, human cytomegalovirus (CMV) promoter as described, for example, in Miller et al. (1989) Biotechniques 7(9):980-990, adenovirus promoters, such as the adenoviral major late promoter, polyoma virus promoters, human T-cell lymphotropic virus (HTLV) promoters, thymidine kinase (TK) promoters such as the Herpes Simplex thymidine kinase promoter, respiratory syncytial virus (RSV) promoters, and B19 parvovirus promoters. Examples of promoters also include other inducible promoters such as the MMT promoter, the metallothionein promoter; heat shock promoters, the albumin promoter, the ApoAI promoter, human globin promoters, and human growth hormone promoter. Other illustrative promoters include immediate-early cytomegalovirus (CMV) enhancer-promoter and CAG hybrid promoter, which combines the CMV enhancer with chicken β-actin (CBA) promoter. See, e.g., McClements et al., Transl. Res. 2013, 161(4):10.1016/j.trsl.2012.12.007. Other examples of promoters include human synapsin-1 (hSYN) promoter, neuron specific enolase (NSE) promoter, and platelet derived growth factor (PDGF) promoter. See, e.g., Shevisova et al., Exp. Physiol. 2004, 90(1)53-59. Other suitable promoters include regulatable promoters, such as Hif1a, doxycycline-activatable promoters, or the promoter for Clq or any other complement protein that correlates with disease. The selection of a suitable promoter will be apparent to those skilled in the art.

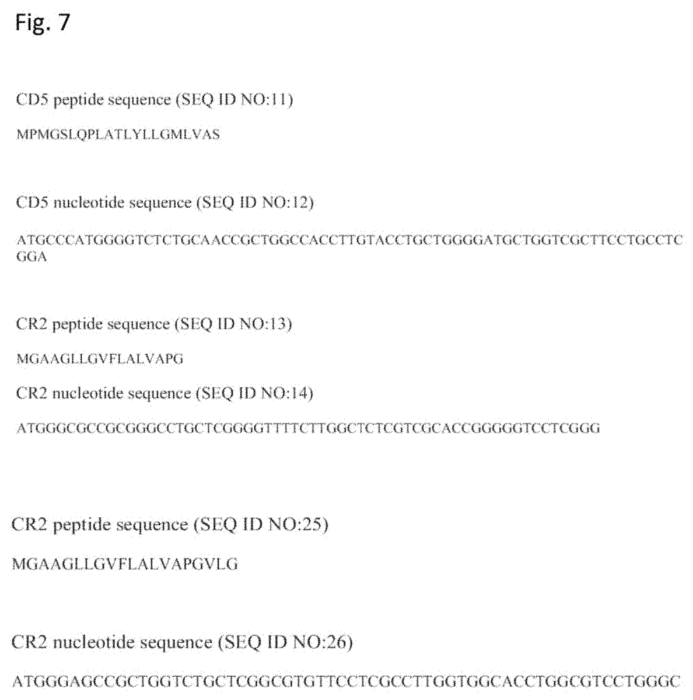

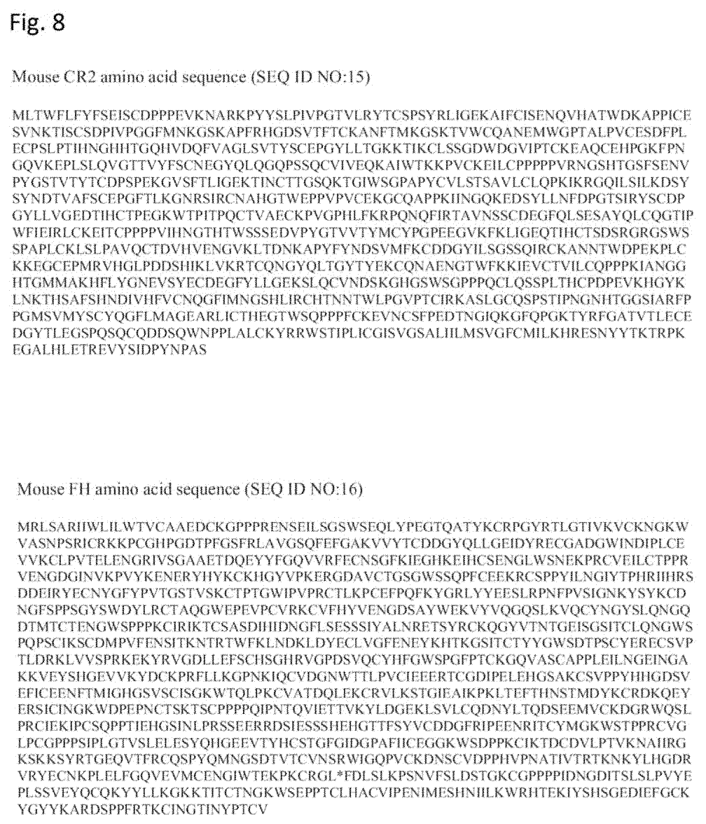

In some embodiments, the CR2-FH fusion protein encoded by the recombinant vector comprises a sequence encoding a signal peptide operably linked at the 5′ end of the sequence encoding the CR2-FH fusion protein. Exemplary nucleotide sequences of signal peptides are provided in FIG. 7 (SEQ ID NO: 12, 14, and 25). In some embodiments, a linker sequence is used for linking the CR2 portion and the FH portion. In some embodiments, the recombinant vector operably encodes a CR2-FH fusion protein having an amino acid sequence of SEQ ID NO: 3. In some embodiments, the recombinant vector operably encodes a CR2-FH fusion protein having an amino acid sequence that is at least about any of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of any of SEQ ID NO: 3. SEQ ID NO: 21, and SEQ ID NO: 23. In some embodiments, the recombinant vector operably encodes a CR2-FH fusion protein comprising at least about any of 400, 450, 500, 550, or more contiguous nucleotides of any of SEQ ID NO: 4, SEQ ID NO:22, and SEQ ID NO: 24. In some embodiments, the recombinant vector comprises a sequence any of SEQ ID NO: 4, SEQ ID NO: 22, and SEQ ID NO: 24. In some embodiments, the recombinant vector comprises a sequence that is at least about any of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the nucleic acid sequence any of SEQ ID NO: 4, SEQ ID NO: 22, and SEQ ID NO: 24. In some embodiments, the recombinant vector comprises least about any of 1200, 1300, 1400, 1500, 1600, or more contiguous nucleotides any of SEQ ID NO: 4, SEQ ID NO: 22, and SEQ ID NO: 24. The recombinant vector may further include a sequence encoding a secretory signal sequence to secret the fusion protein into a medium. The polynucleotide encoding a secretory signal sequence include, for example, a polynucleotide encoding the signal sequence of CD5 or a polynucleotide sequence encoding the signal sequence of CR2.

### Adeno-Associated Virus (AAV) VECTOR

Adeno-associated virus (AAV), a member of the Parvoviridae family, is a small single-stranded DNA virus which infects humans and some other primate species. AAV can infect both dividing and quiescent cells. AAV may exist in an extrachromosomal state without integrating into the genome of the host cell, or it may exhibit site-specific integration on human chromosome 19 (Ali et al. (1994), supra, p. 377). The ability to integrate into the genome of the some target cells (Hirata et al. 2000, J. of Virology 74:4612-4620) allows long-term transgene expression in transduced cells, making AAV vectors convenient for use in gene therapy. AAV vectors have been used in retinal gene therapy to achieve expression of the RPE65 protein in human subjects (Maguire et al., New England J. Med., 2008, 358(21 ):2240-2248; Bainbridge et al., New England J. Med., 2008, 358(21):2231-2239: Hauswirth et al., Human Gene Ther., 2008 19(10):979-990).

A preferred recombinant virus for use in the method of gene therapy described herein is a recombinant adeno-associated virus (AAV) vector operably encoding a CR2-FH fusion protein including, without limitation, any of the CR2-FH fusion protein embodiments described herein, as well as CR2-FH variants as described herein. The recombinant AAV vector is employed as a gene therapy vector ta deliver the CR2-FH fusion protein to a subject afflicted with an eye disease. The disclosure also provides pharmaceutical compositions that include the recombinant AAV vector, and methods for making and using the recombinant AAV vector. Illustrative recombinant AAV vectors include vectors based on serotypes AAV1, AAV2, AAV4, AAV5, and AAV8.

Suitable promoters for use in driving expression of a CR2-FH fusion protein from a recombinant AAV vector include, without limitation, ubiquitous promoters such as immediate-early cytomegalovirus (CMV) enhancer-promoter and CAG hybrid promoter, which combines the CMV enhancer with chicken β-actin (CBA) promoter, as well as tissue- or cell-specific promoters. See, e.g., McClements et al., 2013. Representative examples of promoters for use in AAV vectors include a chicken β-actin (CBA) promoter, a human synapsin-1 (hSYN) promoter, a neuron specific enolase (NSE) promoter, and a platelet derived growth factor (PDGF) promoter (see, e.g., Shevtsova et al., Exp. Physiol. 2004, 90(1)53-59) as well as regulatable promoters, such as Hif1a, doxycycline-activatable promoters, or the promoter for Clq or other complement protein. Illustrative promoters specific for retinal pigment epithelium (RPE) cells include, for example, RPE65 promoter and VMD2 promoter. See, e.g., McClements et al., 2013. Promoters specific for retinal ganglion cells (RGC) or Muller cells are also suitable. Expression in RGC or Muller cells of a CR2-FH fusion protein or derivative thereof that contains structural elements that promote secretion of the fusion protein from the RBC or Muller cells may permit diffusion of the fusion protein to the outer retina, thereby effectuating treatment for AMD.

In some embodiment, the recombinant AAV vector is a recombinant serotype 2 (AAV2) vector. In some embodiments, the recombinant AAV2 vector is a mutant AAV2 having one or more mutations in a capsid protein. An illustrative mutation is a tyrosine (Y) to phenylalanine (F) mutation. A recombinant AAV2 vector having one or more Y to F mutations in a capsid protein may be referred to as a “YF mutant.” A suitable recombinant AAV2 vector can have one, two, three, four or more YF mutations in one or more capsid proteins. In some embodiments, the YF mutation is at a surface-exposed amino acid residue. An illustrative recombinant AAV2 vector is a quadruple YF mutant capsid AAV2 vector. Suitable promoters for use in driving expression of a CR2-FH fusion protein from a recombinant AAV2 vector include, without limitation, chicken β-actin (CBA) promoter and hSYN promoter. An illustrative recombinant AAV2 vector is a quadruple YF mutant capsid AAV2 vector comprising a CBA promoter operably linked to a nucleotide sequence encoding a CR2-FH fusion protein, so as to permit expression of the fusion protein in a host cell, such as a cell of a retina, for example, a retinal ganglion cell (RGC). The CR2-FH fusion protein encoded by the recombinant AAV2 vector can encode, without limitation, any of the CR2-FH fusion protein embodiments described herein as well as CR2-FH variants as described herein. A preferred CR2-FH fusion protein encoded by the recombinant AAV2 vector has amino acid sequence SEQ ID NO:21. A preferred recombinant AAV2 vector comprises a nucleotide sequence having SEQ ID NO:22. A triple Y-F mutant capside recombinant AAV2 vector may be particularly well suited for treatment of glaucoma.

In some embodiments, the recombinant AAV vector is a recombinant serotype 5 (AAV5) vector. Examples of promoters for use in driving expression or a CR2-FH fusion protein from a recombinant AAV5 vector include, without limitation, vitelliform macular dystrophy 2 (VMD2) promoter, RPE65 promoter, or OA1 promoter. An illustrative recombinant AAV5 vector includes a VMD2 promoter operably linked to a nucleotide sequence encoding a CR2-FH fusion protein, so as to permit expression of the fusion protein in a host cell, such as u cell of a retina, for example, a retinal pigment epithelium (RPE) cell. A preferred CR2-FH fusion protein encoded by the recombinant AAV2 vector has amino acid sequence SEQ ID NO:21. A preferred recombinant AAV2 vector comprises a nucleotide sequence having SEQ ID NO:22. A recombinant AAV5 vector may be well suited for treatment of macular degeneration (MD), including age-related macular degeneration (AMD), particularly in treatment methods that target RPE cells.

In some embodiments, the recombinant AAV vector is a recombinant AAV variant SbH10 vector or recombinant AAV serotype 6(AAV6) vector. AAV variant ShH10 is closely related to AAV serotype 6 (AAV6). Promoters described herein may be suitable for use in driving expression of a CR2-FH fusion protein from a recombinant AAV variant ShH10 vector or recombinant AAV serotype 6 (AAV6). See, for example, Klimezak et al., PLoS One. 2009 Oct. 14; 4(10):e7467.

The disclosure also provides a pharmaceutical composition that includes a recombinant vector, such as an AAV vector, operably encoding a CR2-FH fusion protein, as well as methods for making the recombinant vector.

### Pharmaceutical Compositions

Also provided is a pharmaceutical composition comprising a recombinant vector, such as a recombinant AAV2 or AAV5 vector, operably encoding a CR2-FH fusion protein, and a pharmaceutically acceptable carrier. The pharmaceutical composition, in some embodiments, comprises a recombinant vector operably encoding a CR2-FH fusion protein comprising: a) a CR2 portion comprising a CR2 or a fragment thereof, and b) a FH portion comprising a FH or a fragment thereof, including, without limitation, any of the CR2-FH fusion protein embodiments described herein, as well as CR2-FH variants as described herein The pharmaceutical composition may be suitable for a variety of modes of administration described herein, including for example systemic or localized administration. Local delivery of the fusion proteins of the present disclosure using gene therapy is advantageous in that it provides the therapeutic agent to the target area, for example to the eye or the eye tissue.

The pharmaceutical compositions can be in the form of injectable solutions. The pharmaceutical compositions described herein can be packaged in single unit dosages or in multidosage forms.

In some embodiments, the pharmaceutical compositions comprise a recombinant vector operably encoding a CR2-FH fusion protein, and a pharmaceutically acceptable carrier, which is suitable for administration to human. In some embodiments, the pharmaceutical compositions comprise a recombinant vector operably encoding a CR2-FH fusion protein and a pharmaceutically acceptable carrier suitable for intraocular injection. In some embodiments, the pharmaceutical compositions comprise a recombinant vector operably encoding a CR2-FH fusion protein and a pharmaceutically acceptable carrier suitable for intravenous injection.

The compositions are generally formulated as sterile, substantially isotonic, and in full compliance with all Good Manufacturing Practice (GMP) regulations of the U.S. Food and Drug Administration. In some embodiments, the composition is free of pathogen. For injection, the pharmaceutical composition can be in the form of liquid solutions, for example in physiologically compatible buffers such as Hank's solution or Ringer's solution. In addition, the pharmaceutical composition can be in a solid form and redissolved or suspended immediately prior to use. Lyophilized compositions are also included.

The present disclosure in some embodiments provides compositions comprising a recombinant vector operably encoding a CR2-FH fusion protein and a pharmaceutically acceptable carrier suitable for administration to the eye. Such pharmaceutical carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Suitable pharmaceutical excipients include starch, glucose, lactose, sucrose, gelatin, mall, rice, sodium state, glycerol monostearate, glycerol, propylene, water, and the like. The pharmaceutical composition, if desired, can also contain minor amounts of welling or emulsifying agents, or pH buffering agents. The recombinant vector and other components of the composition may be encased in polymers or fibrin glues to provide controlled release of the fusion protein. These compositions can take the form of solutions, suspensions, emulsions, ointment, gel, or other solid or semisolid compositions, and the like. The compositions typically have a pH in the range of 4.5 to 8.0. The compositions must also be formulated to have osmotic values that are compatible with the aqueous humor of the eye and ophthalmic tissues. Such osmotic values will generally be in the range of from about 200 to about 400 milliosmoles per kilogram of water (“mOsm/kg”), but will preferably be about 300 mOsm/kg.

In some embodiment, the composition is formulated in accordance with routine procedures as a pharmaceutical composition adapted for injection. Typically, compositions for injection are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions may further comprise additional ingredient, for example preservatives, buffers, tonicity agents, antioxidants and stabilizers, nonionic wetting or clarifying agents, viscosity-increasing agents, and the like.

Suitable preservatives for use in a solution include polyquaternium-1, benzalkonium chloride, thimerosal, chlorobutanol, methyl paraben, propyl paraben, phenylethyl alcohol, edetate disodium, sorbic acid, benzethonium chloride, and the like. Typically (but not necessarily) such preservatives are employed at a level of from 0.001% to 1.0% by weight.

Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and preferably, between about pH 7 and pH 7.5.

Suitable tonicity agents are dextran 40, dextran 70, dextrose, glycerin, potassium chloride, propylene glycol, sodium chloride, and the like, such that the sodium chloride equivalent of the ophthalmic solution is in the range 0.9 plus or minus 0.2%.

Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxmethylpropylcellulose, lanolin, methylcellulose, petrolatum, polyethylene glycol, polyvinyl alcohol, polyvinylpyrrolidone, carboxymethylcellulose and the like.

A pharmaceutical composition for gene therapy (i.e., for delivery of a recombinant vector operably encoding a CR2-FH fusion protein) can be in an acceptable diluent, or can comprise a slow release matrix in which the gene delivery vehicle, such as the recombinant vector, is imbedded. Alternatively, where the complete gene delivery system can be produced intact from recombinant cells, e.g., retroviral vectors, the pharmaceutical composition can comprise one or more cells which produce the gene delivery system.

In clinical settings, a gene delivery system for a delivery of a therapeutic protein such as a CR2-FH fusion protein can be introduced into a subject by any of a number of methods. For instance, a pharmaceutical composition of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the subject being localized. For example, the gene delivery vehicle can be introduced by catheter, See U.S. Pat. No. 5,328,470, or by stereotactic injection, Chen et al. (1904), Proc. Natl. Acad. Sci., USA 91:3054-3057. A polynucleotide encoding a CR2-FH fusion protein can be delivered in a gene therapy construct by electroporation using techniques described, Dev et al. (1994), Cancer Treat. Rev. 20:105-115.

In some embodiments, there is provided a pharmaceutical composition for gene delivery to the eye. Ophthalmic solutions useful for storing and/or delivering expression vectors have been disclosed, for example, in WO 03/077796A2.

### Treatment Methods

A CR2-FH fusion protein can be delivered to a subject by expression of the CR2-FH fusion protein to vivo, which is often referred to as “gene therapy”. Recombinant DNA techniques for delivering a CR2-FH fusion protein to a host cell involve, in simplified form, inserting a CR2-FH encoding polynucleotide into an appropriate vector, inserting the vector into an appropriate host cell, and expressing the vector in the host cell. Typically, the host cell into which the recombinant vector is introduced is present in the subject, and the recombinant vector is delivered to the host cell of the subject to vivo. However, in some embodiments, host cells may be transfected with a recombinant vector operably encoding for the CR2-FH fusion protein ex vivo, and the transfected cells are then provided to a subject to be treated with the fusion protein. In some embodiments, the host cells to be transfected are obtained from the subject. Such methods are well-known in the art.

The disclosure provides methods of treating eye diseases associated with FH deficiencies including, for example, glaucoma and macular degeneration, including age-related macular degeneration.

“Treating” or “to treat” a disease is defined as delivering one or more CR2-FH fusion proteins, including in situ delivery via gene therapy, to a subject, with or without other therapeutic agents, in order to palliate, ameliorate, stabilize, reverse, slow, delay, prevent, reduce, or eliminate either the disease, or a symptom of the disease, or to retard or stop the progression of the disease or a symptom of the disease. Treatment can be prophylactic, administered prior to development or detection of a disease in a subject, or it can be therapeutic, administered after development or detection of a disease in a subject. The subject may, or may not, be diagnosed with a disease. The term “disease” is inclusive of any pathological condition experienced by the patient, whether by infection, injury, autoimmune activity, etc., without limitation. An “effective amount” is an amount sufficient to treat a disease.

An “individual” or “subject” is a vertebrate, preferably a mammal, more preferably a human. Mammals include, but are not limited to, farm animals, sport animals, pets, primates, mice and rats. In some embodiments, the individual is human. In some embodiments, the individual is an individual other than human. In some embodiments, the individual is an animal model for the study of a disease in which the alternative complement pathway is implicated. Individuals amenable to treatment include those who are presently asymptomatic but who are at risk of developing a symptomatic macular degeneration-related disorder at a later time. For example, human individuals include those having relatives who have experienced such a disease, and those whose risk is determined by analysis of genetic or biochemical markers, by biochemical methods, or by other assays such as T cell proliferation assay. In some embodiments, the individual is a human leaving a mutation or polymorph in its FH gene that indicates an increased susceptibility to develop a disease in which alternative complement pathway is implicated (such as age-related macular degeneration). In some embodiments, the individual has a wildtype or protective haplotype of FH. Different polymorphs of FH have been disclosed in US Pat. Pub. No. 2007/0020647, which is incorporated herein in its entirety.

The gene therapy treatment methods described herein can be used as primary treatment modalities for eye disease, or they may be employed as secondary or adjunct therapies. When employed in combination with other treatments, administration of the recombinant vectors described herein can occur prior to, concurrent with, or subsequent to one or more other treatments.

### Treatment of Glaucoma

In one embodiment, the treatment method involves treatment of a subject afflicted with, or at risk for, glaucoma. Glaucomas are a group of progressive optic neuropathies characterized by degeneration of retinal ganglion cells and resulting changes in the optic nerve head. A major risk factor for glaucoma is raised intraocular pressure that compresses and damages the optic nerve. Altered regulation of complement factor H may be present in glaucoma, and complement Clq and C3 upregulation has been implicated in glaucomatous neurodegeneration. Luo et al., Investig. Ophthamol. Visual Sci. (2010) 51:2671: Stasi et al., Investig. Ophthamol. Visual Sci. (2006) 47: 1024; Tezel et al., Investig. Ophthamol. Visual Sci. (2010) 51:5071. Mirzael, Sci Rep (2017) 7: 12685; Yang et al., Investig. Ophthamol. Visual Sci. (2015) 56: 5816; and Kuehn, et al. Exp. Eye Res. (2006) 83: 620-628.

The present disclosure provides methods of treating glaucoma by administering an effective amount of a recombinant vector operably encoding a CR2-FH fusion protein, or a composition comprising the recombinant vector, wherein the CR2-FH fusion protein comprises: a) a CR2 portion comprising a CR2 or a fragment thereof, and b) a FH portion comprising a FH or a fragment thereof, including, without limitation, any of the CR2-FH fusion protein embodiments described herein, as well as CR2-FH variants as described herein. In some embodiments, the FH or fragment thereof comprises at least the first four N-terminal short consensus repeat (SCR) domains of FH. In some embodiments, the CR2 portion of the CR2-FH fusion protein is capable of binding to a CR2 ligand, and the FH portion of the CR2-FH fusion protein is capable of inhibiting complement activation of the alternative pathway.

In one embodiment, the recombinant vector administered to a subject to treat glaucoma is an adeno-associated virus (AAV) vector, preferably a recombinant AAV2 vector. In some embodiments, the recombinant AAV2 vector for use in treating glaucoma is a mutant AAV2 having one or more mutations in a capsid protein. In some embodiments, the recombinant AAV2 vector is a tyrosine capsid-mutant AAV2 vector; an illustrative mutation is a tyrosine (Y) to phenylalanine (F) mutation. In some embodiments, the recombinant AAV2 vector for use in treating glaucoma can have one, two, three, four or more tyrosine to phenyl alanine (YF) mutations in one or more capsid proteins. In some embodiments, the YF mutation is at a surface-exposed capsid tyrosine residue. A preferred recombinant AAV2 vector for use in treating glaucoma is a quadruple YF mutant capsid AAV2 vector. The recombinant AAV2 vector includes a promoter for driving expression of a CR2-FH fusion protein. In some embodiments, the recombinant AAV2 vector for use in treatment of glaucoma includes a chicken β-actin (CBA) promoter or a hSYN promoter. A preferred vector for use in treatment of glaucoma is quadruple YF mutant capsid AAV2 vector comprising a CBA promoter operably linked to a nucleotide sequence encoding a CR2-FH fusion protein, so as to permit expression of the fusion protein in a host cell, such as a cell of a retina, for example, a retinal ganglion cell (RGC).

In some embodiments, the recombinant AAV2 vector is administered to one or both eyes of the subject, for example, by intravitreal injection.

The recombinant AAV2 vector is administered to a subject in one dose and a titer optimized to target CR2-FH transduction to RGCs and other inner retinal cells, avoid immune detection of the vector, and cause a therapeutic effect in the subject. In some embodiments, the vector is administered to a subject in one or more doses of at least about about 3×10⁸ vg/mL, 3×10⁹ vg/mL, 3×10¹⁰ vg/mL, 3×10¹¹ vg/mL, 3×10¹² vg/mL, or 3×10¹³ vg/mL;

In some embodiments, the amount of recombinant vector administered to the subject is effective to treat or prevent one or more aspects or symptoms of glaucoma. For example, in some embodiments, administration of the recombinant AAV2 to a subject is effective to slow, halt, or reverse the loss of retinal ganglion cells (RGC) in the subject. In some embodiments, administration of the recombinant AAV2 to a subject is effective to slow, halt, or reverse the loss of retinal ganglion cells (RGC) in the subject. In some embodiments, administration of the recombinant AAV2 to a subject is effective to slow, halt, or reverse RGC axon degeneration in the subject. In some embodiments, administration of the recombinant AAV2 to a subject is effective to slow, halt, or reverse damage to optic nerves of the subject. In some embodiments, administration of the recombinant AAV2 to a subject is effective to slow, halt, or reverse the progression of glaucoma in the subject.

Treatment of Age-Related Macular degeneration (AMD)

In one embodiment, the treatment method involves treatment of a subject afflicted with, or at risk for, macular degeneration, such as age-related macular degeneration (AMD). AMD is clinically characterized by progressive loss of central vision which occurs as a result of damage to the photoreceptor cells in an area of the retina culled the macula. AMD has been broadly classified into two clinical states; a wet form and a dry form, with the dry form making up to 80-90% of total cases. The dry form is characterized clinically by the presence of macular drusen, which are localized deposits between the retinal pigment epithelium (RPE) and the Bruch's membrane, and by geographic atrophy characterized by RPE cell death with overlying photoreceptor atrophy. Wet AMD, which accounts for approximately 90% of serious vision loss, is associated with neovascularization in the area of the macular and leakage of these new vessels. The accumulation of blood and fluid can cause retina detachment followed by rapid photoreceptor degeneration and loss of vision. It is generally accepted that the wet form of AMD is preceded by and arises from the dry form.

Analysis of the contents of drusen and Bruch's membrane deposits in AMD patients has shown a large number of inflammatory proteins including amyloid proteins, coagulation factors, and a large number of proteins of the complement pathway. A genetic variation in the complement factor H substantially raises the risk of age-related macular degeneration (AMD), suggesting that uncontrolled complement activation underlies the pathogenesis of AMD, Edward et al., Science (2005), 308: 421; Haines et al., Science (2005), 308: 419; Klein et al., Science (2005), 308: 385-389; Hageman et al., Proc. Natl. Acad. Sci. USA (2005), 102: 7227.

The disclosure provides methods of treating macular degeneration, including AMD (such as wet or dry forms of AMD), administering on effective amount of a recombinant vector operably encoding a CR2-FH fusion protein, or a composition comprising the recombinant vector, wherein the CR2-FH fusion protein comprises: a) a CR2 portion comprising a CR2 or a fragment thereof, and b) a FH portion comprising a FH or a fragment thereof, including, without limitation, any of the CR2-FH fusion protein embodiments described herein, as well as CR2-FH variants as described herein. In some embodiments, the FH or fragment thereof comprises at least the first four N-terminal short consensus repeal (SCR) domains of FH. In some embodiments, the CR2 portion of the CR2-FH fusion protein is capable of binding to a CR2 ligand, and the FH portion of the CR2-FH fusion protein is capable of inhibiting complement activation of the alternative pathway.

In one embodiment, the recombinant vector administered to a subject to treat AMD is an adeno-associated vims (AAV) vector, preferably a recombinant AAV5 vector. The recombinant AAV5 vector optionally includes a promoter for driving expression of a CR2-FH fusion protein. The promoter can be a tissue-specific promoter for use in eye tissue, such as retinal pigment epithelium (RPE) tissue or choroid tissue. In some embodiments, the recombinant AAV5 vector for use in treatment of AMD includes a vitelliform macular dystrophy 2 (VMD2) promoter or a RPE65 promoter. A preferred vector for use in treatment of AMD is an AAV5 vector comprising a VMD2 promoter operably linked to a nucleotide sequence encoding a CR2-FH fusion protein, so as to permit expression of the fusion protein in a host cell, such as a cell of a retina, for example, a retinal pigment epithelium (RPE) cell. A recombinant AAV5 vector is well-suited for treatment methods that target RPE cells.

In another embodiment, the recombinant AAV vector administered to a subject to treat AMD is a recombinant AAV variant ShH10 vector or recombinant AAV serotype 6 (AAV6) vector. The recombinant AAV variant ShH10 vector or AAV6 vector optionally includes a promoter for driving expression of a CR2-FH fusion protein. The promoter can be a tissue-specific promoter for use in eye tissue, such as RGC or Muller cells.

In some embodiments, the recombinant AAV5 vector, AAV variant ShH10 vector, or AAV6 vector is administered to an eye of the subject, for example, by injection, such as subretinal injection.

The recombinant AAV5 vector, AAV variant ShH10 vector, or AAV6 vector is administered to a subject in one or more doses in an amount sufficient to cause a therapeutic effect in the subject. In some embodiments, the recombinant AAV5 vector, AAV variant ShH10 vector, or AAV6 vector is administered to a subject in one or more doses of at least about about 3×10⁸ vg/mL, 3×10⁹ vg/mL, 3×10¹⁰ vg/mL, 3×10¹¹ vg/mL, 3×10¹² vg/mL, or 3×10¹³ vg/mL; in some embodiments, the recombinant AAV5 vector, AAV variant ShH10 vector, or AAV6 vector is administered to a subject in one or more doses (e.g., 1 μL dose) or at most about 3×10¹⁵ vg/mL, 3×10¹⁴ vg/mL, 3×10¹³ vg/mL, 3×10¹² vg/mL, 3×10¹¹ vg/mL, or 3×10¹⁰ vg/mL. In some embodiments, the recombinant AAV5 vector, AAV variant ShH10 vector, or AAV6 vector is administered to a subject in one or more doses of between about 3×10⁹ vg/mL and about 3×10¹⁰ vg/mL, or between about 3×10¹⁰ vg/mL and about 3×10¹² vg/mL. In an illustrative treatment method, the recombinant AAV5 vector, AAV variant ShH10 vector, or AAV6 vector is administered to a subject in 1 μL dose of 3×10¹¹ vg/mL.

In some embodiments, the amount of recombinant vector administered to the subject is effective to treat or prevent one or more aspects or symptoms of AMD. For example, in some embodiments, administration of the recombinant AAV5 vector, AAV variant ShH10 vector, or AAV6 vector to a subject is effective to promote retinal reattachment. The extent of retinal reattachment in a subject can be measured monitoring retina reattachment using fundus photography, optical coherence tomography (OCT), or electroretinography, and the method of treatment optionally includes post-treatment monitoring. In some embodiments, administration of the recombinant AAV5 vector, AAV variant ShH10 vector, or AAV6 vector to a subject is effective to slow, halt or reverse choroidal neovascularization (CNV) in a subject and/or reduce CNV-mediated complement activation. In some embodiments, administration of the recombinant vector to a subject is not accompanied by an adverse immune response in the subject. In some embodiments, administration of the recombinant AAV5 vector, AAV variant ShH10 vector, or AAV6 vector slows or halts the progression of macular degeneration in the subject.

More generally, in some embodiments the amount of recombinant vector administered to the subject is effective to treat or prevent one or more aspects or symptoms of AMD including, but not limited to, formation of ocular drusen, inflammation in the eye or eye tissue, loss of photoreceptor cells, loss of vision (including for example visual acuity and visual field), neovascularization (such as choroidal neovascularization or CNV), and retinal detachment. In some embodiments, the amount of recombinant vector administered to the subject is effective to treat or prevent one or more aspects or symptoms of AMD including photoreceptor degeneration, RPE degeneration, retinal degeneration, chorioretinal degeneration, cone degeneration, retinal dysfunction, retinal damage in response to light exposure (such as constant light exposure) or oxidative stress (smoking, diet, etc), damage of the Bruch's membrane, loss of RPE function, loss of integrity of the histoarchitecture of the cells and/or extracellular matrix of the normal macular, loss of function of the cells in the macula, photoreceptor dystrophy, mucopolysaccharidoses, rod-cone dystrophies, cone-rod dystrophies, anterior and posterior uvitis, or diabetic neuropathy.

In some embodiments, there are provided methods of treating macular degeneration (such as age-related macular degeneration or AMD) in an individual, comprising administering to the individual an effective amount of a composition comprising a recombinant vector operably encoding a CR2-FH fusion protein comprising: a) a CR2 portion comprising a CR2 or a fragment thereof, and b) a FH portion comprising a FH or a fragment thereof, including, without limitation, any of the CR2-FH fusion protein embodiments described herein, as well as CR2-FH variants as described herein. In some embodiments, the disease to be treated is a dry form of AMD. In some embodiments, the disease to be treated is a wet form of AMD.

In some embodiments, there are provided methods of treating (such as reducing, delaying, eliminating, or preventing) formation of drusen in the eye of an individual, comprising administering to the individual an effective amount of a composition comprising a recombinant vector operably encoding a CR2-FH fusion protein comprising: a) a CR2 portion comprising a CR2 or a fragment thereof, and b) a FH portion comprising a FH or a fragment thereof, including, without limitation, any of the CR2-FH fusion protein embodiments described herein, as well as CR2-FH variants as described herein.

In some embodiments, there are provided methods of treating (such as reducing, delaying, eliminating, or preventing) inflammation in the eye of an individual, comprising administering to the individual an effective amount of a composition comprising a recombinant vector operably encoding a CR2-FH fusion protein comprising: a) a CR2 portion comprising a CR2 or a fragment thereof, and b) a FH portion comprising a FH or a fragment thereof, including, without limitation, any of the CR2-FH fusion protein embodiments described herein, as well as CR2-FH variants as described herein.

In some embodiments, there are provided methods of treating (such as reducing, delaying, eliminating, or preventing) loss of photoreceptors cells in an individual, comprising administering to the individual an effective amount of a composition comprising a recombinant vector operably encoding a CR2-FH fusion protein comprising: a) a CR2 portion comprising a CR2 or a fragment thereof, and b) a FH portion comprising a FH or a fragment thereof, including, without limitation, any of the CR2-FH fusion protein embodiments described herein, as well as CR2-FH variants as described herein.

In some embodiments, there are provided methods of treating (such as reducing, delaying, eliminating, or preventing) loss of photoreceptors cells in an individual, comprising administering to the individual an effective amount of a composition comprising a recombinant vector operably encoding a CR2-FH fusion protein comprising: a) a CR2 portion comprising a CR2 or a fragment thereof, and b) a FH portion comprising a FH or a fragment thereof, including, without limitation, any of the CR2-FH fusion protein embodiments described herein, as well as CR2-FH variants as described herein.

AMD is characterized by choroidal neovascularization (CNV). In some embodiments, there are provided methods of treating (such as reducing, delaying, eliminating, or preventing) neovascularization associated with AMD, comprising administering to the individual an effective amount of a composition comprising a recombinant vector operably encoding a CR2-FH fusion protein comprising: a) a CR2 portion comprising a CR2 or a fragment thereof, and b) a FH portion comprising a FH or a fragment thereof, including, without limitation, any of the CR2-FH fusion protein embodiments described herein, as well as CR2-FH variants as described herein.

In some embodiments, there are provided methods of treating (such as reducing, delaying, eliminating, or preventing) retinal detachment associated with AMD, comprising administering to the individual an effective amount of a composition comprising a recombinant vector operably encoding a CR2-FH fusion protein comprising: a) a CR2 portion comprising a CR2 or a fragment thereof, and b) a FH portion comprising a FH or a fragment thereof, including, without limitation, any of the CR2-FH fusion protein embodiments described herein, as well as CR2-FH variants as described herein.

In some embodiments, there are provided methods of improving (including for example decreasing, delaying, or blocking loss of) visual acuity or visual field in the eye of an individual, comprising administering to the individual an effective amount of a composition comprising a recombinant vector operably encoding a CR2-FH fusion protein comprising: a) a CR2 portion comprising a CR2 or a fragment thereof, and b) a FH portion comprising a FH or a fragment thereof, including, without limitation, any of the CR2-FH fusion protein embodiments described herein, as well as CR2-FH variants as described herein.

In addition to macular degeneration and glaucoma, other eye diseases that can be treated by methods of the present disclosure include, for example, retinitis pigmentosa, diabetic retinopathy, optic neuritis, and other eye diseases that involve a local inflammatory process. In some embodiments, the eye disease is diabetic retinopathy. In some embodiments, the eye disease is retinitis pigmentosa.

### Methods of Administration

The compositions described herein can be administered to an individual via any effective route. Typically, the composition is administered directly to the eye or the eye tissue. In some embodiments, the compositions are administered by injection to the eye (intraocular injection) or to the tissues associated with the eye. The compositions can be administered, for example, by intraocular injection, periocular injection, subretinal injection, intravitreal injection, trans-septal injection, subscleral injection, intrachoroidal injection, intracameral injection, subconjuntival injection, sub-Tenon's injection, retrobulbar injection, peribulbar injection, or posterior justascleral delivery. These methods are known in the art. For example, for a description of exemplary periocular routes for retinal drug delivery, see Periocular routes for retinal drug delivery, Raghava et al. (2004), Expert Opin. Drug Deliv. 1(1):99-114. The compositions may be administered, for example, to the vitreous, aqueous humor, sclera, conjunctiva, the area between the sclera and conjunctiva, the retina choroids tissues, macula, or other area in or proximate to the eye of an individual. The compositions can also be administered to the individual as an implant. Preferred implants are biocompatible and/or biodegradable sustained release formulations which gradually release the compounds over a period of time. Ocular implants for drug delivery are well-known in the art. See, e.g., U.S. Pat. Nos. 5,501,856, 5,476,511, and 6,331,313. The compositions can also be administered to the individual using iontophoresis, including, but are not limited to, the ionophoretic methods described in U.S. Pat. No. 4,454,151 and U.S. Pat. App. Pub. Nos. 2003/0181531 and 2004/0058313.

The optimal effective amount of the compositions can be determined empirically and will depend on the type and severity of the disease, route of administration, disease progression and health, mass and body area of the individual. Such determinations are within the skill of one in the art. The effective amount can also be determined based on in vitro complement activation assays.

One of skill in the art skilled person can easily determine the therapeutically effective amount of recombinant AAV to administer to a subject by routine trials, or by constructing dose-response curves. An administration of 10³, 10⁴, 10⁵, 10⁶, 10⁷, 10⁸, 10⁹, 10¹⁰, 10¹¹ or more recombinant AAV viral panicles may be a mutable dose. In some embodiments, the recombinant AAV vector is stably integrated into the genome of the host cell and provides long term (at least 4-8 weeks, preferably at least 8-12 weeks, more preferably at least 6 months or lifelong) expression of the CR2-FH fusion protein.

The compositions may be administered in a single dose, or in multiple doses. The viral particles may be administered by injection in various locations in the eye or tissue associated with the eye, such as intraocular, intravitreal, subretinal, periocular, subconjunctival, or sub-tenon.

The pharmaceutical compositions comprising the recombinant vectors described herein can be administered alone or in combination with other molecules known to have a beneficial effect on retinal attachment or damaged retinal tissue, including molecules capable of tissue repair and regeneration and/or inhibiting inflammation. Examples of useful cofactors include anti-VEGF agents (such as an antibody against VEGF), basic fibroblast growth factor (bFGF), ciliary neurotrophic factor (CNTF), axokine (a mutein of CNTF), leukemia inhibitory factor (LIF), neutrotrophin 3 (NT-3), neurotrophin-4 (NT-4), nerve growth factor (NGF), insulin-like growth factor II, prostaglandin E2, 30 kD survival factor, taurine, and vitamin A. Other useful cofactors include symptom-alleviating cofactors, including antiseptics, antibiotics, antiviral and antifungal agents and analgesics and anesthetics.

### EXAMPLES

### Example 1

### Examples of Sequences of CR2-FH Fusion Proteins and Signal Peptides

CR2-FH fusion proteins can be readily made using recombinant DNA cloning and gene expression methods known to the art.

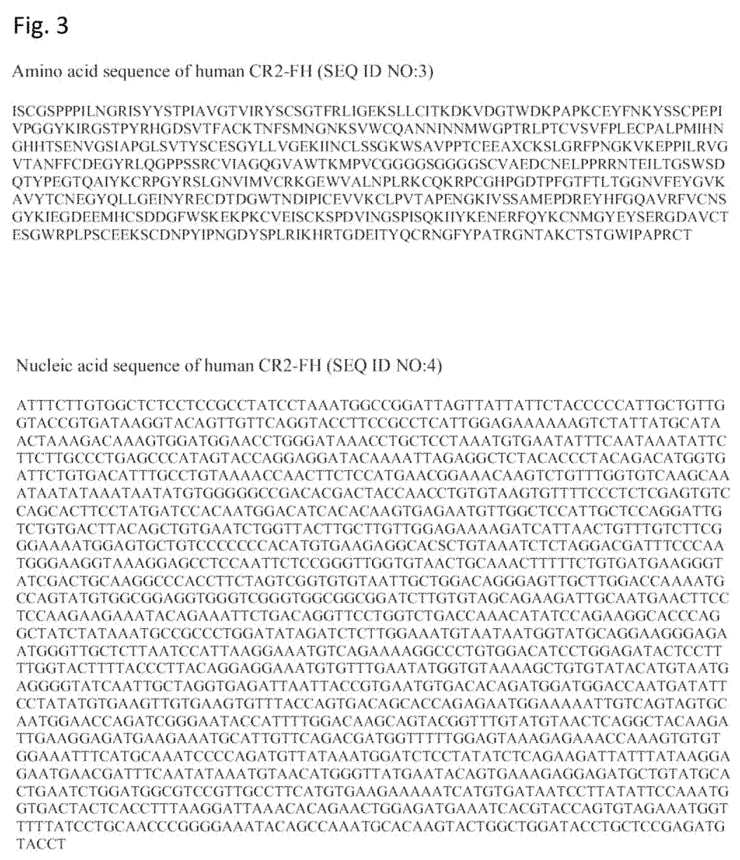

FIG. 3 provides an illustrative amino acid sequence for a human CR2-FH fusion protein (SEQ ID NO: 3) having a linker region between the CR2 and FH portions, and a polynucleotide encoding the fusion protein (SEQ ID NO: 4).

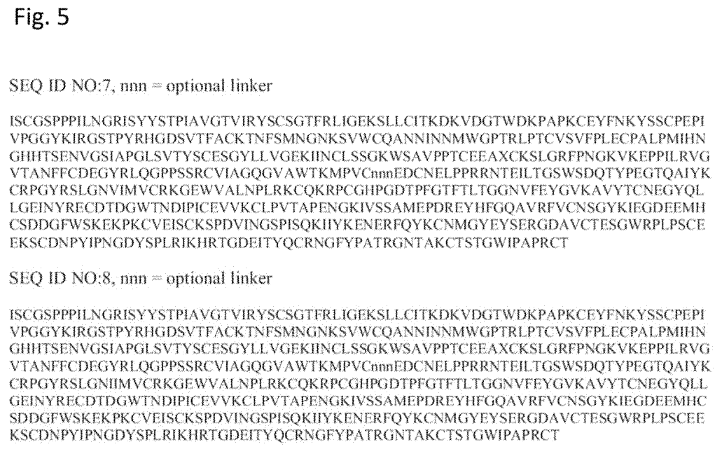

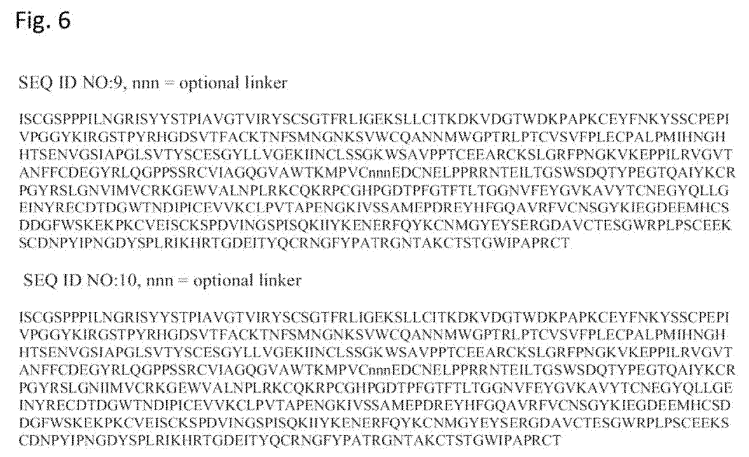

FIGS. 4-6 provide illustrative amino acid sequences of additional illustrative CR2-FH fusion proteins (SEQ ID NOs: 5-10); “nnn” represents an optional linker.

FIG. 7 provides illustrative amino acid sequences of signaling peptides (SEQ ID NOs: 1 and 13) and polynucleotides encoding the signaling peptides (SEQ ID NOs: 12 and 14).

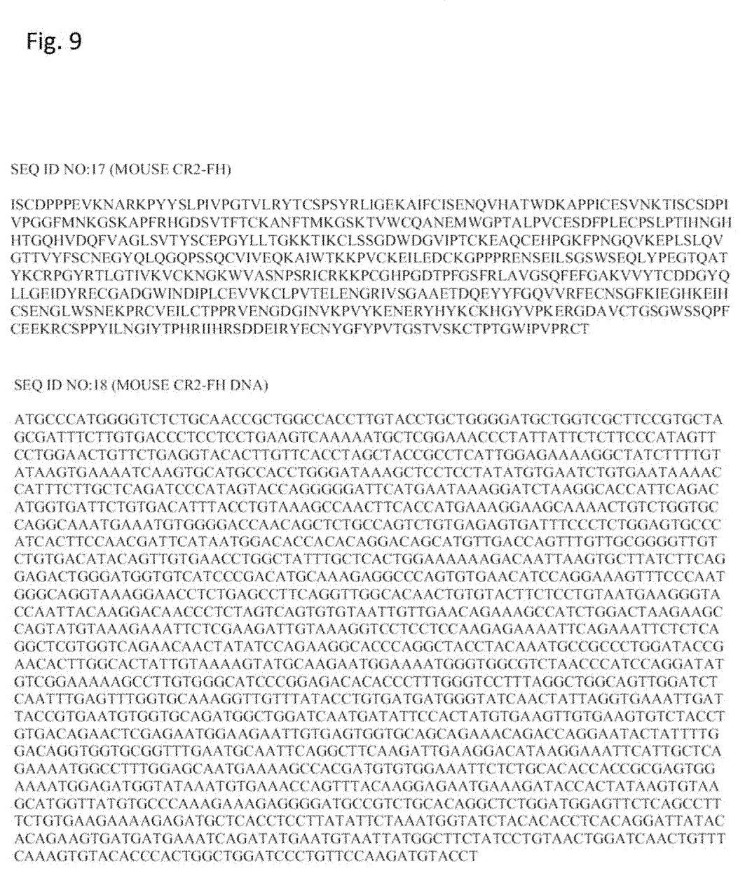

A mouse CR2-FH fusion protein can, in some embodiments, include the first four SCR domains of CR2 and the first five SCR domains of FH, with or without a linker. FIG. 9 provides the amino acid sequence or CR2-fH (also known as CR2NLFH, where “NL” indicates no linker) (SEQ ID NO:17) and a polynucleotide that encodes a mouse CR2-FH plus the signal peptide (SEQ ID NO:18). See U.S. Pat. No. 7,739,304.

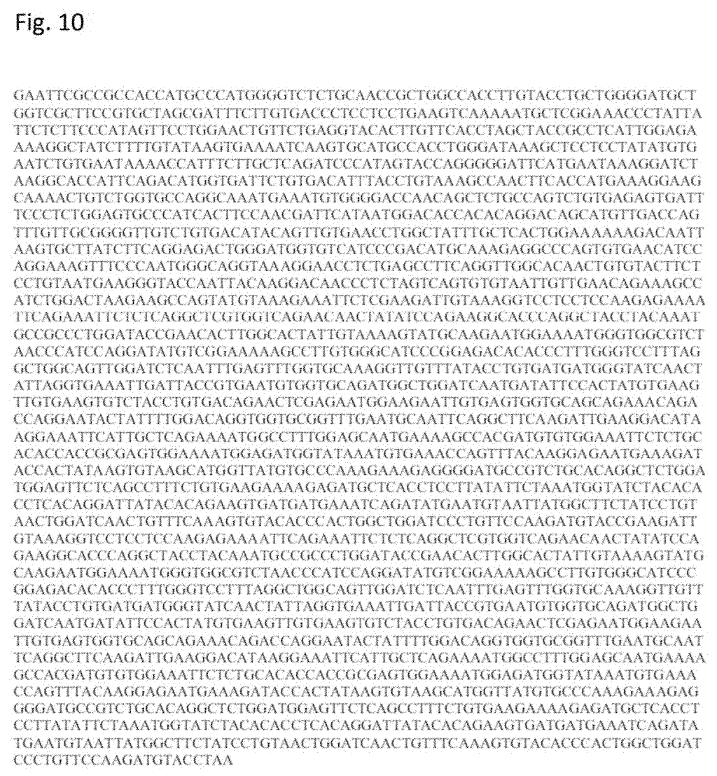

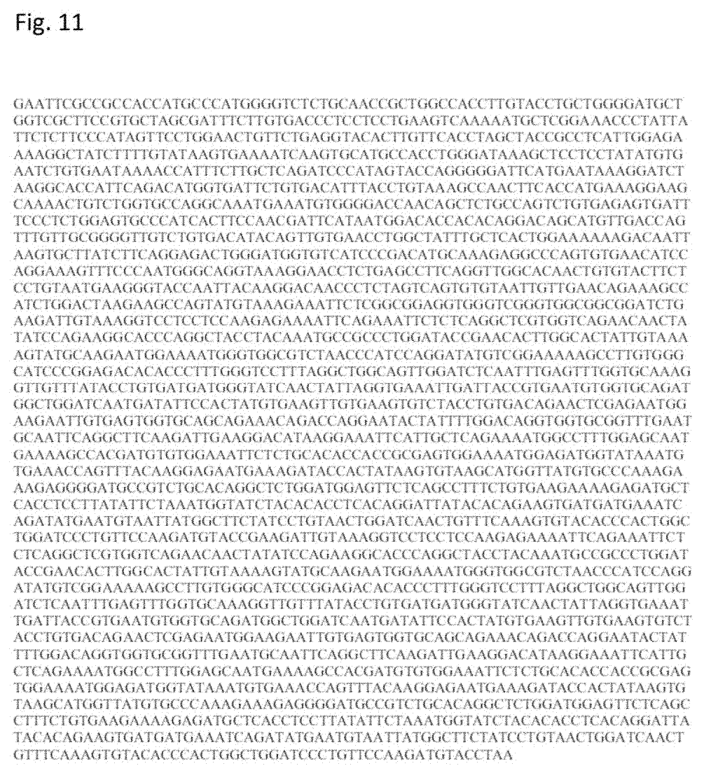

A mouse CR2-FH fusion protein can, in some embodiments, include a CR2 portion that includes the first four SCR domains of CR2 and two tandemly linked FH portions, each containing the first five SCR domains of FH. FIG. 10 provides the DNA sequence of CR2NLFHFH (“NL” indicates no linker), a mouse CR2-FH fusion protein containing a CR2 portion and two FH portions without a linker sequence (SEQ ID NO: 19). FIG. 11 provides the DNA sequence of CR2LFHFH (“L” indicates linker), also known as CR2-fH2 or CR2-fHH, a mouse CR2-FH fusion protein containing a CR2 portion linked to two FH portions via a linker sequence (SEQ ID NO:20). See U.S. Pat. No. 7,759,304.

FIG. 12 provides an amino acid sequence of a human CR2-FH fusion protein (designated as human CR2-fH or CR2fH) (SEQ 1D NO:21) and a polynucleotide that encodes a human CR2-fH plus the signal peptide (SEQ ID NO:22). See U.S. Pat. No. 7,759,304.

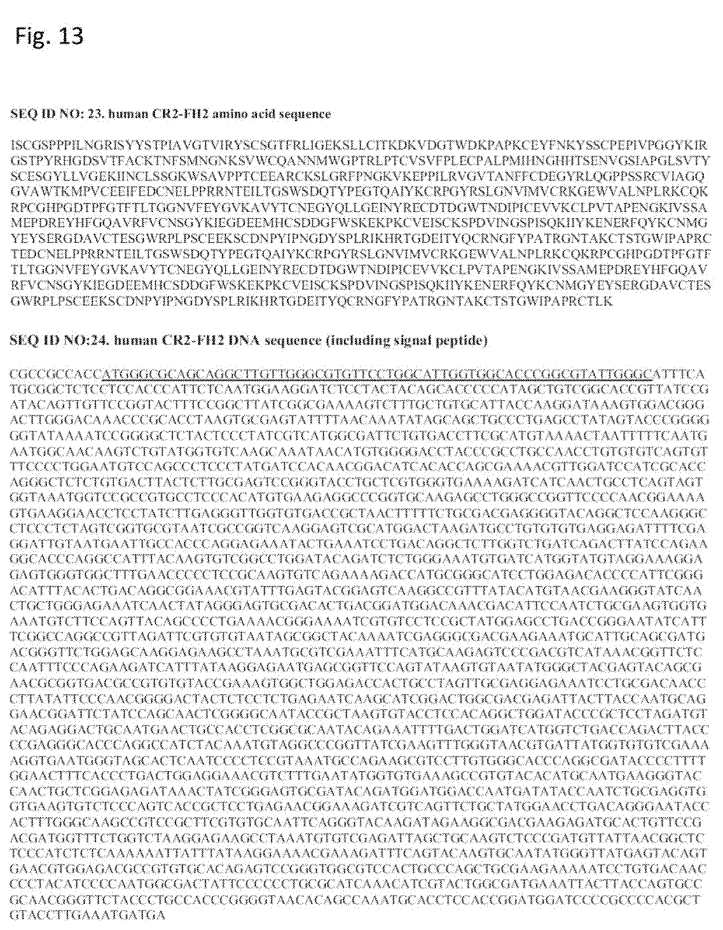

FIG. 13 provides on amino acid sequence of a human CR2-FH fusion protein containing two FH portions (designated as human CR2-FH2 or CR2fH2) (SEQ ID NO:23) and a polynucleotide that encodes a human CR2-FH2 plus the signal peptide (SEQ ID NO:24).

### Example 2

### Inhibiting Complement C3 Activation by Gene Therapy Reduces Glaucoma Progression

Complement activation is associated with glaucoma, and precedes neurodegeneration in animal models. Steele et al., Investig. Ophthamol. Visual Sci. (2006) 47:977. Howell et al., J. Clinic. Invest. (2011) 121; 1429. Knockout of the classic pathway initiator Clq delays disease in DBA/2J (D2) mice. Howell et al., J. Clinic. Invest. (2011) 121: 1429. Howell et al., Neurobiol. Disease (2014) 71:44. All three complement activation pathways converge at C3 cleavage. The therapeutic effect of limiting C3 activation during glaucoma progression in DBA/2J mice was tested using ocular gene therapy.

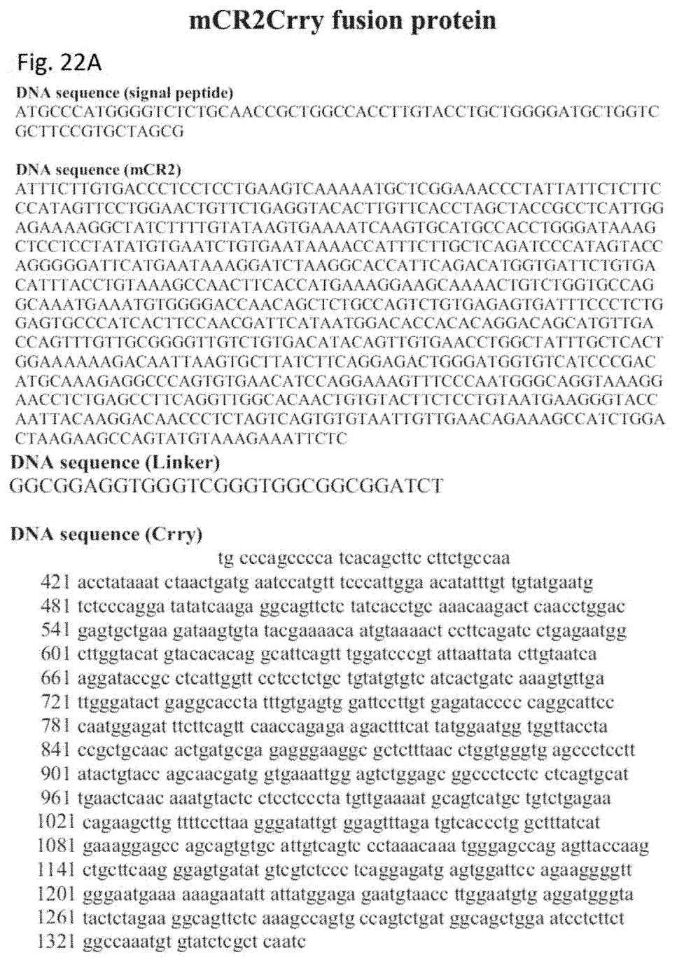

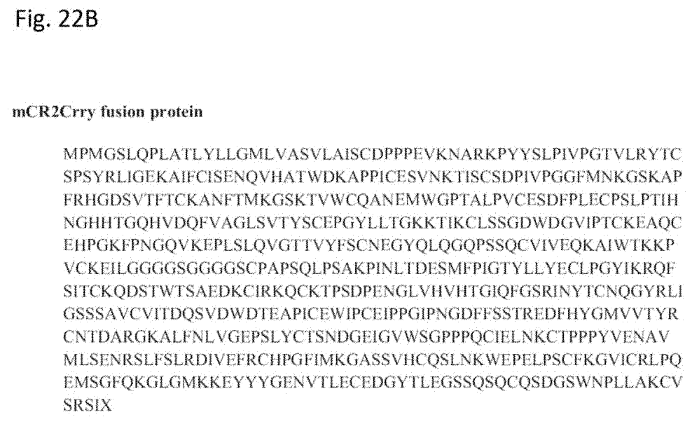

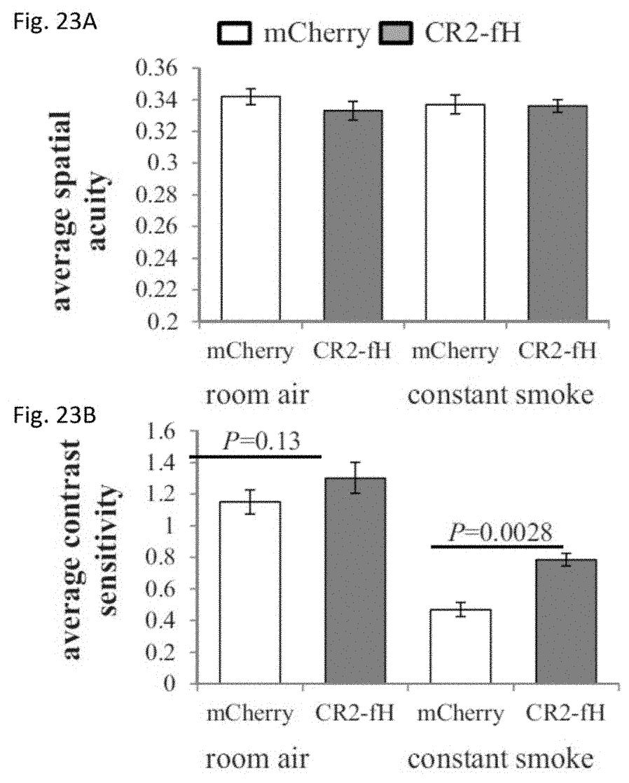

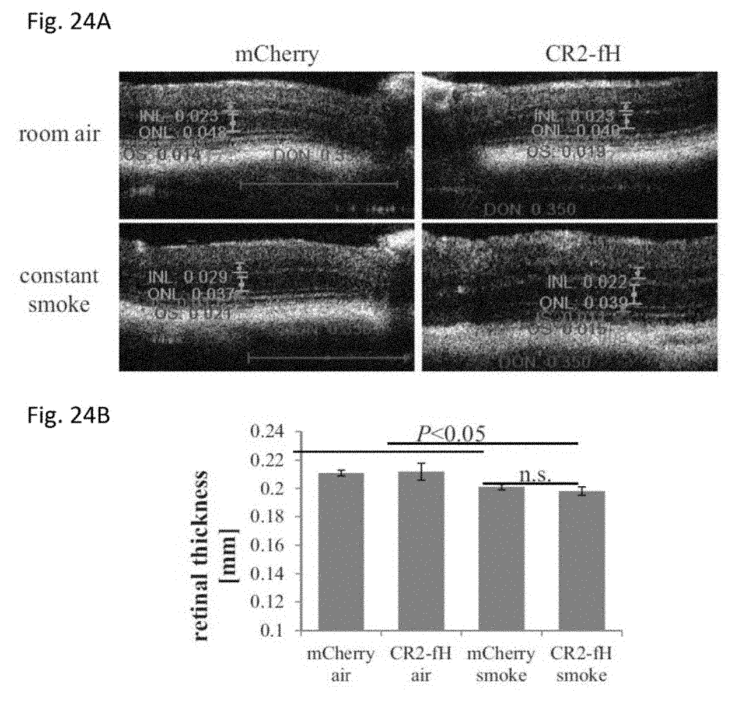

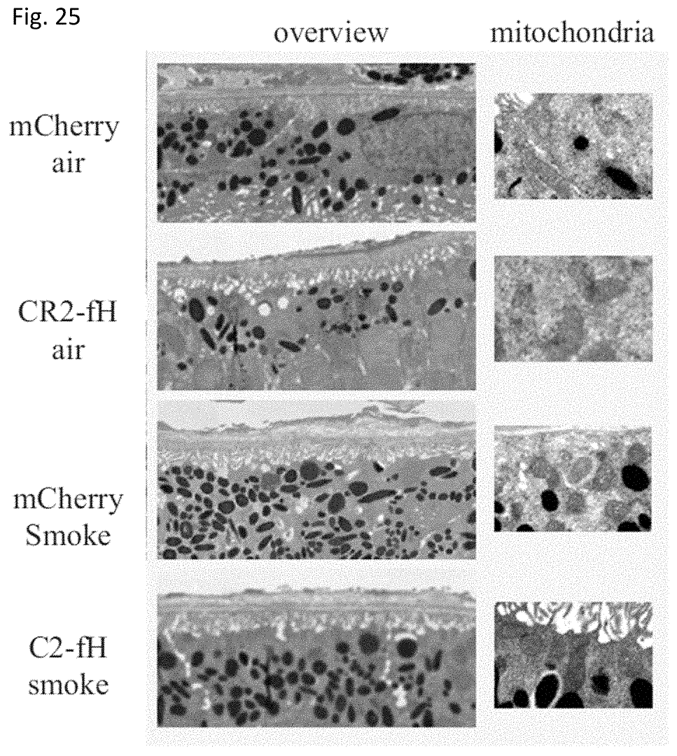

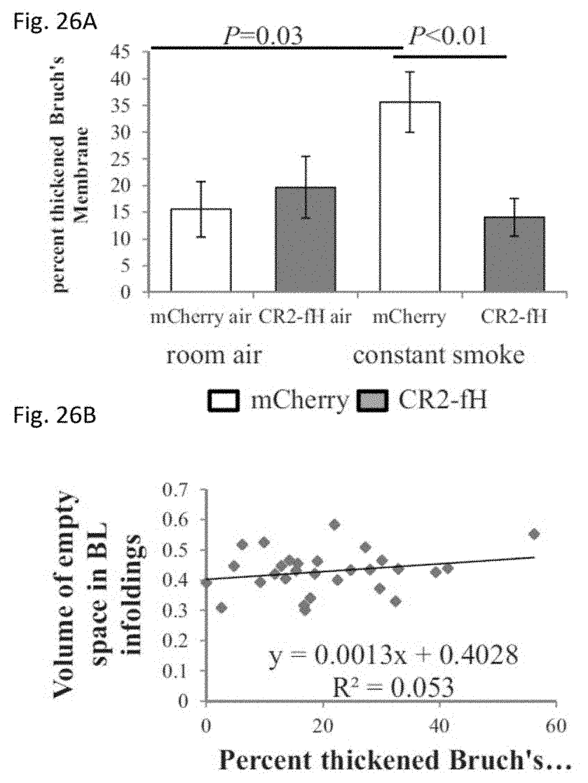

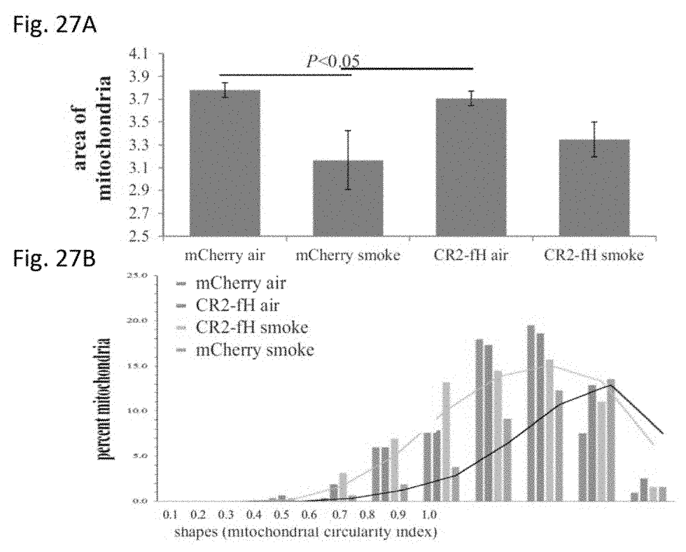

Methods. CR2-Crry was utilized (see FIG. 22), which is the soluble rodent-specific complement inhibitor (sCrry) linked to a complement receptor 2 (CR2) targeting moiety that directs sCrry to sites or C3b-fragments (iC3b/C3dg/C3d) deposition. Murine Crry is analogous to the human CR1 protein. Human CR1 protein, like murine Crry, is a C3 convertase inhibitor which blocks all complement pathways, whereas CR-2fH blocks only the alternative pathway (AP). CR2-sCrry was packaged using triple YF mutant capsid AAV2 vector with chicken β-actin (CBA) promoter. AAV2-CR2-Crry or control AAV2-GFP was delivered by bilateral intravitreal injection in 7-month old DBA/2J female mice, an age that precedes detectable damage in RGC axons and optic nerves. At 10 and 12 months, RGC and axonal density were quantified in confocal images of retinal wholemounts immunostained for RGC and axonal markers (Brn3b, pNF), and optic nerve damage was scored as mild, moderate or severe by proportion of degenerative/lost axons (<10%, 10-50% or >50%, respectively). Naive 5-7 month-old DBA/2J retinal wholemounts were immunostained for C3d, Brn3b and pNF. One-way ANOVA for Brn3b/pNF counts was conducted by group/age, followed by Student's t-test. Naïve, AAV2-CR2-Crry and AAV2-GFP-treated retinas from 10 month-old DBA/2J were analyzed by quantitative, reverse PCR to measure the relative fold change of total Crry expression.

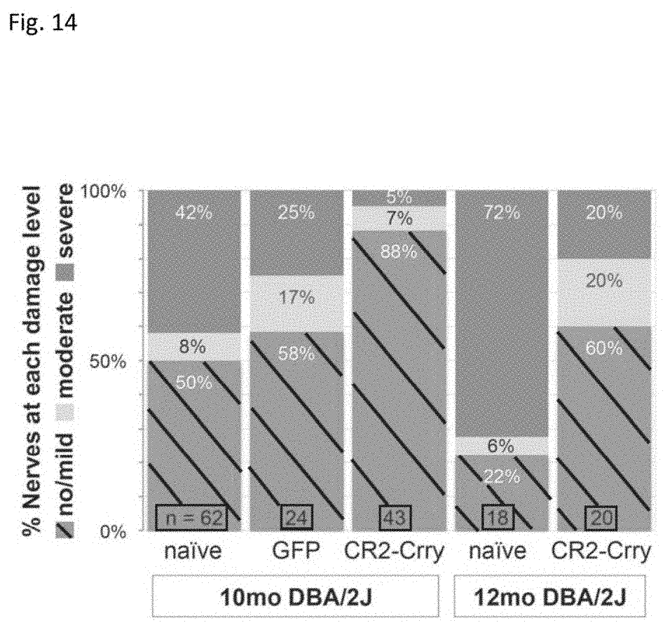

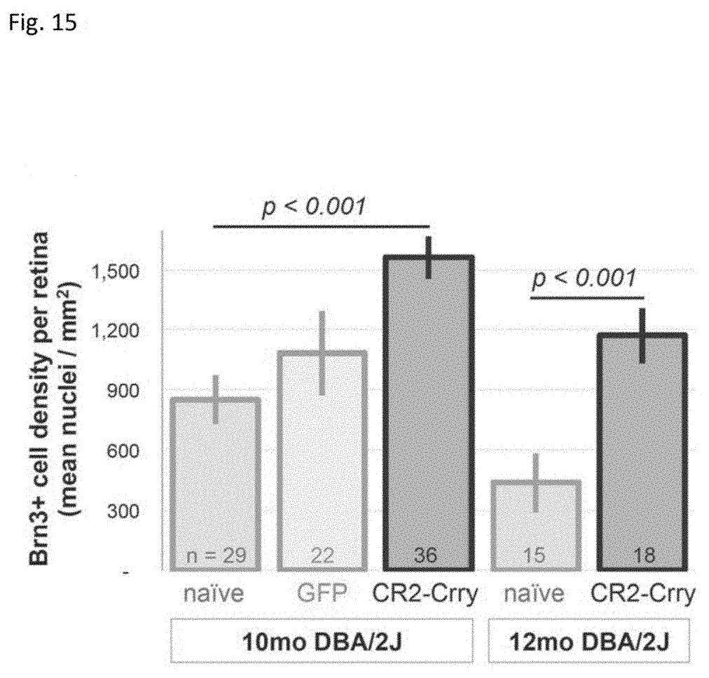

Results. Preceding therapy, 5-7 month-old D2 showed C3d-stained RGC somata, dendrites and axons. At 10 and 12 months, AAV-CR2-Crry-treated DBA/2J mice maintained 38% and 40% more healthy optic nerves respectively, and had 34% and 57% fewer severely damaged nerves at each age, compared with naïve DBA/2J (FIG. 14). AAV-GFP had a modest effect, with 1% more healthy and 54% less severe nerves relative to 10 month old naïve DBA/2J. AAV-CR2-Crry-treated retinas showed uniformly intact and bundled intraretinal axons at 750-μm eccentricity, with 3.3 axons/fascicle at 10 months of age (p=0.014) and 2.6 at 12 months of age (p>0.001), compared to variably dystrophic and depleted fascicles in age-matched naive mice (2.6 and 1.5 axons), and in 10 month old AAV-GFP controls (2.4 axons). AAV2-CR2-Crry suppressed RGCs loss, showing 84% higher Brn3b-nuclei density at 10 months (p=0.001) and 168% at 12 months (p=0.001), whereas 10 month old AAV-GFP-treated eyes only increased RGC density by 27% (FIG. 15). At 10 months, AAV-CR2-Crry-treated DBA/2J retinas expressed an 8-fold mean increase in Crry mRNA expression (p>0.05) relative to AAV2-GFP and naïve retinas.

Viral gene therapy targeted to block C3 activation provided a high-precision and potent neuroprotective strategy to reduce the advance of chronic glaucoma.

### Example 3

### Delivery of CR2-fH Using AAV Vector Therapy as a Treatment Strategy in Mouse Model of Choroidal Neovascularization

Complement activation plays a significant role in age-related macular degeneration (AMD) pathogenesis, and polymorphisms interfering with factor H (fH) function, a complement alternative pathway (AP) inhibitor, are associated with increased AMD risk. An AP inhibitor, a fusion protein consisting of a complement receptor-2 fragment linked to the inhibitory domain of fH (CR2-fH) was previously validated as an efficacious treatment for choroidal neovascularization (CNV) when delivered intravenously. AAV-mediated delivery (AAV5-VMD2-CR2-fH AAV5-VMD2-mCherry) was tested as an alternative by subretinally injecting 2 month-old C57BL/6J mice. Secretion of CR2-PH was confirmed in polarized retinal pigment epithelium (RPE) cells. A safe concentration or AAV5-VMD2-CR2-fH was identified using electroretinography, optical coherence tomography (OCT), RPE morphology and antibody profiling. CNV was induced using argon laser photocoagulation at 3 months-of-age, and (OCT) assessment demonstrated reduced CNV with AAV5-VMD2-CR2-fH administration. Bioavailability studies using dotblot showed gene-therapy delivered similar CR2-fH to the RPE/choroid as those provided by intravenous injections, and C3a ELISA verified reduced CNV-associated ocular C3a production. These results illustrated the importance of the AP of complement in CNV development and its potential role in AMD treatment.

In order to reduce the number of invasive ocular injections received by exudative or “wet” age-related macular degeneration (AMD) patients, gene therapy approaches are being investigated to serve as an alternative or potential adjunct therapy to standard of care. These trials were triggered by the successful application of AAV gene therapy in Leber's Congenital Amauosis (reviewed in Pierce et al. 2015). Meier et al. (2017) have reported safety and potential efficacy in a phase 1 trial using AAV2-mediated expression of soluble Flt-1, an endogenously expressed and secreted VEGF inhibitor that binds VEGF-A2 (NCT01024998). Other clinical trials testing AAV-mediated ocular gene therapy currently listed on ClinicalTrials.gov include X-linked Retinitis Pigmentosa (AAV-RPGR: NCT03116113). Choroideremia (AAV.REP1; NCT01461213) and Leber's Hereditary Optic Neuropathy (LHON) (scAAV2-P1ND4v2; NCT02161380) among others, and basic science research is focusing on gene delivery in addition diseases, testing efficacy in animal models of Stargardt disease (Han et al., 2012), primary open angle glaucoma (Li et al., 2009), and others.

Multiple complement pathway polymorphisms have been linked with an increased risk of developing AMD. These include genetic variants to complement component 3 (C3) (Yates et al., 2007), complement component 2 (C2) (Gold et al, 2006), complement component 9 (C9) (Seddon et al., 2013), convenient factor 1 (CF1) (Fagerness et al., 2009; Fritsche et al., 2016), complement factor B (CFB) (Gold et al., 2006), and vitronectin (VTN) (referenced in Tan et al., 2016). The most abundant polymorphism identified however, occurs with a mutation of Y402H in factor H (CFH), and thereby poses the greatest single genetic risk for AMD (reviewed in Tan et al., 2016). The complement system is part of the innate and adaptive immune system, and functions as an early response system activated at sites of injury either directly or by natural antibody binding to stress and/or injury-exposed antigens. The pathway triggers the production of different biological effector molecules, anaphylatoxins, opsonins and the membrane attack complex (MAC), that are involved in recruitment of phagocytes, opsonization of damaged cellular material, and lysis of cells, respectively (Muller-Eberhard, 1988). Importantly, upon activation, complement amplification by the complement alternative pathway (AP) results in pathologically high levels of complement activation products and the generation of a pro-inflammatory micro-environment. CFH, a soluble AP inhibitor, is found abundantly in human blood (Pangburn et al., 2009) and can be secreted from various cell types including RPE cells (Kim et al., 2009). As CFH plays an essential role in complement regulation (Muller-Eberhard, 1988), mutations to this gene are expected to result in deregulation of the alternative pathway of complement activation Rodriguez et al., 2014)¹⁴. The use of a targeted inhibitor to the AP, CR2-fH, in mouse models of complement-dependent pathology, choroidal neovascularization (CNV) and smoke-induced ocular pathology have been described (Rohrer et al., 2009; Wooded et al., 2016). CR2-fH comprises a targeting domain (fragment of complement receptor 2, CR2) and a complement inhibitory domain (short consensus repeats 1-5 fragment of fH). As CR2 binds iC3b, C3dg, and C3d, cell bound opsonins that are present at sites of complement activation, the CR2 domain targets the inhibitor fH to sites of complement activation, where it is needed to inhibit complement (reviewed in Holers et al., 2013). Additionally, Cashman et al. (2015) have shown that a soluble form of the complement inhibitor CD59, when overexpressed via AAV2 injected either into the subretinal space (i.e., targeting RPE), or injected intravitreally (targeting Muller cells), reduced choroidal neovascularization in mouse.

The safety and efficacy of subretinal administrations of an AAV vector encoding the CR2-fH inhibitor were investigated in the mouse CNV model. The AAV5 serotype was chosen due to its ability to target the retinal pigment epithelium (RPE) and photoreceptors (Pang et al., 2008) as well as its ability to drive gene expression by 7 days post injection (Kong et al., 2010). RPE-specific gene expression has also been ensured by use of the RPE-specific promoter VMD2 (Esumi et al., 2004).

### AAV5-Mediated CR2-fH Expression in the Mouse RPE

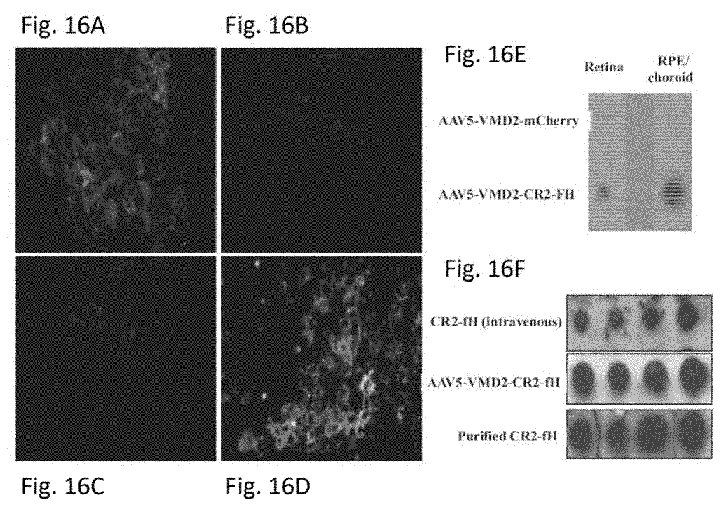

One month following subretinal injections of AAV5 vectors, flatmounts of RPE/choroid eyecups were imaged either for mCherry fluorescence (FIG. 16A, FIG. 16C) or stained for the CR2 portion of the fusion protein (FIG. 16B, FIG. 16D). As shown previously by Kong and colleagues (2010), RPE staining for marker genes are present at 1 month after injection, with mCherry autofluorescence visible in the AAV5-VMD2-mCherry injected eyes (FIG. 16A) and CR2-immunofluorescence delectable in the RPE of AAV5-VMD2-CR2-fH injected eye (FIG. 16D). Dotblot analysis staining for anti-CR2 demonstrated the presence of CR2-PH in RPE/choroid and to a lesser extent in the retina of mice injected with AAV5-VMD2-CR2-fH (FIG. 16E).

To get an estimate of whether the amount of CR2-fH present in the RPE/choroid is comparable to that obtained after tail vein injections of a therapeutic dose (250 μg/animal; Rohrer et al., 2009), RPE/choroid samples were probed for the presence of CR2-fH. Since CR2-fH will only bind to sites of injury, but not to healthy tissue (Alawieh et al., 2016), animals with 3-day old CNV lesions, the time point with maximal complement activation (Rohrer et al., 20U9) were compared. Dotblots of RPE/choroid extract dilution series of CNV animals treated with CR2-fH protein when compared to injected with AAV5-VMD2-CR2-fH revealed comparable amounts of CR2-fH (FIG. 16F). CR2-fH was delectable in both retina and RPE/choroid fractions of injected mice, indicative of secretion into both the subretinal and choroidal space as suggested by the cell-culture experiments.

### Evaluation of Retinal Structure and Morphology After Subretinal AAV5-VMD2-CR2-fH Injection

In order to monitor retinal morphology and function owing to AAV5-mediated gene transfer of mCherry and CR2-fH, all mice received ophthalmic examination including optokinetic responses (OKR), electroretinography (c-waves and focal ERG) and optical coherence tomography (OCT). In addition, RPE/choroid flatmounts were analyzed for RPE morphology. Lack of immunogenicity or the secretable fusion protein CR2-fH was confirmed, testing for antibody formation.

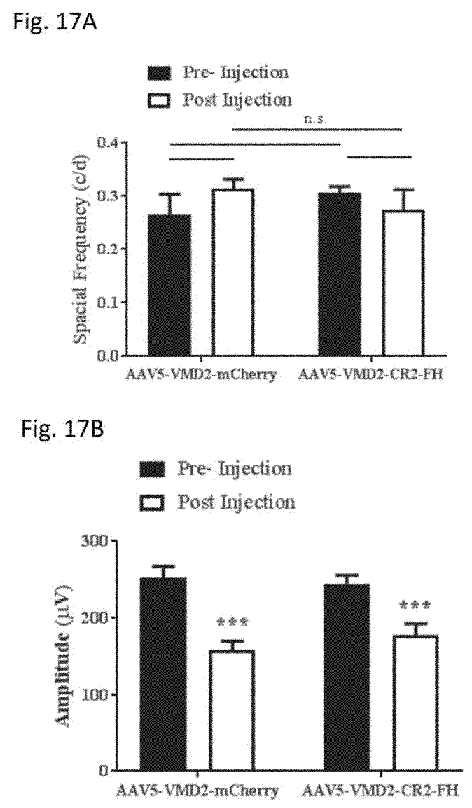

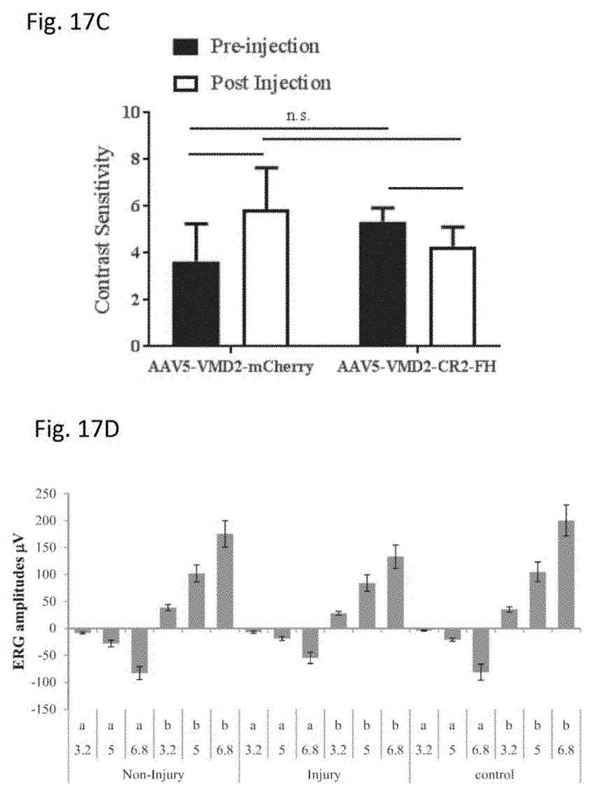

Spatial acuity and contrast sensitivity were analyzed by OKR, to determine the effect of retinal detachment and reattachment on cone visual function. Due to the location of the detachment (temporal retina) and the nature of the OKR response (the stimulus sweeps from temporal to nasal), AAV5-VMD2-CR2-mCherry and AAV5-VMD2-CR2-fH mice were to determined to have lost approximately 20% of their visual acuity, and contrast sensitivity was impaired (FIG. 17A, FIG. 17B). However, there was no difference between the two groups. Using full-field electroretinography, c-wave amplitudes, which originate from the RPE were assessed. Significant attenuated c-wave amplitudes were observed, consistent with damage during the needle penetration and retinal detachment, but no gene-specific effects were observed (P=0.65) (FIG. 17C). Finally, to distinguish between the effect of the local lesion and the retinal detachment of the reduction in function, focal ERGs were analyzed in regions proximal and distal to the lesions in AAV5-VMD2-CR2-mCherry and AAV5-VMD2-CR2-fH mice (FIG. 17D). As predicted, focal ERG responses obtained in the regions proximal to the lesion were significantly reduced (80%), whereas recorded in distal regions were found to return to almost normal levels (20% reduction). Again, no gene-specific effects were observed.

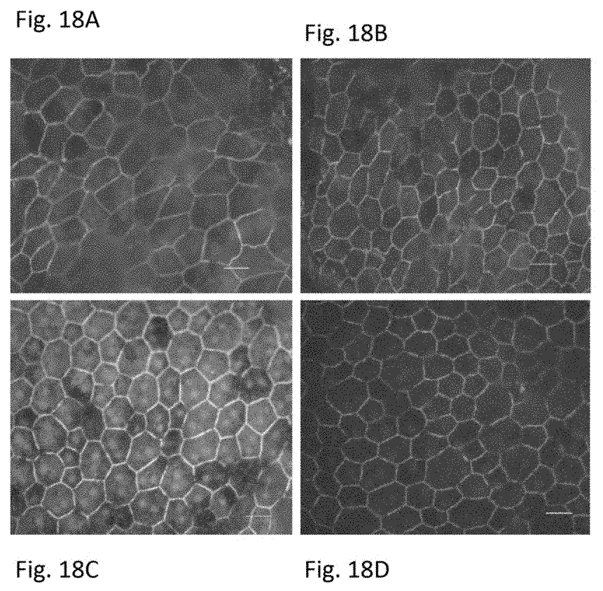

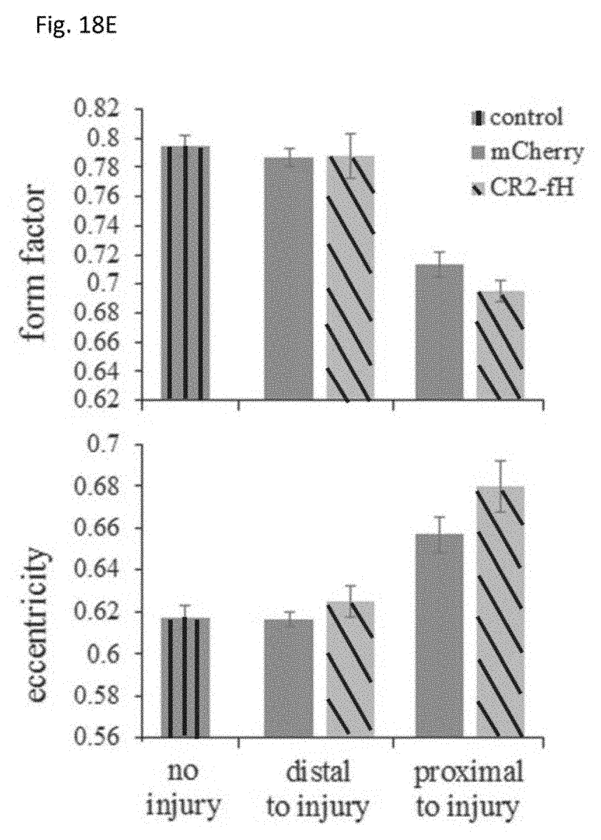

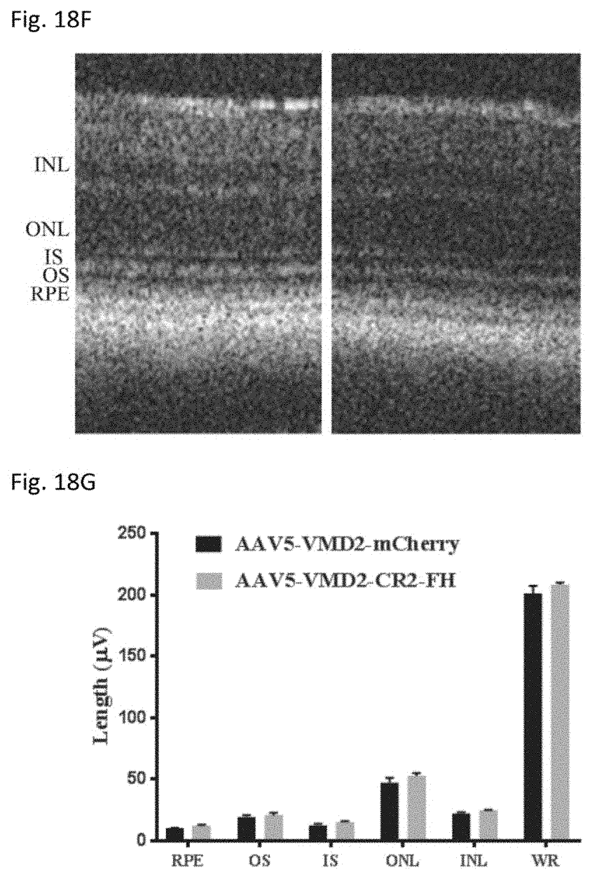

These functional changes correlated with structural alteration in the RPE. RPE/choroid flatmounts were analyzed for RPE cell morphology using the cell junction marker ZO-1. In all animals, ZO-1 staining revealed a halo of unhealthy RPE cells surrounding the injection site (FIG. 18A, FIG. 18D). ‘Unhealthy’ was defined as cells having lost their normal hexagonal shape based on eccentricity (assessing eccentricity of an ellipse) and form factor (equals 1 for a perfectly circular object) (CellProfiler software). Healthy mouse RPE cells from naïve age-matched animals exhibit a form factor of ˜0.79 and an eccentricity value of ˜0.62. RPE cells located within close proximity to the lesion site (area: 1.22±0.12 μm²; based on area of retina being ˜15.6 μm² (Remtulla et al., 1985) this represent ˜8.0%) exhibit reduced form factor and elevated eccentricity values, whereas RPE cells outside the lesion site exhibit normal values indistinguishable of those from naive animals (FIG. 18E). As for all the other readouts, no gene-specific effects were observed. The retina beneath the healthy RPE was unaffected as shown by OCT. The thickness of the individual layers (RPE, outer segments [OS], inner segments [IS], outer nuclear layer [ONL], inner nuclear layer [INL] and whole retina [WR]) were indistinguishable between animals injected with AAV5-VMD2-CR2-mCherry or AAV5-VMD2-CR2-fH (FIG. 18F, FIG. 18G).

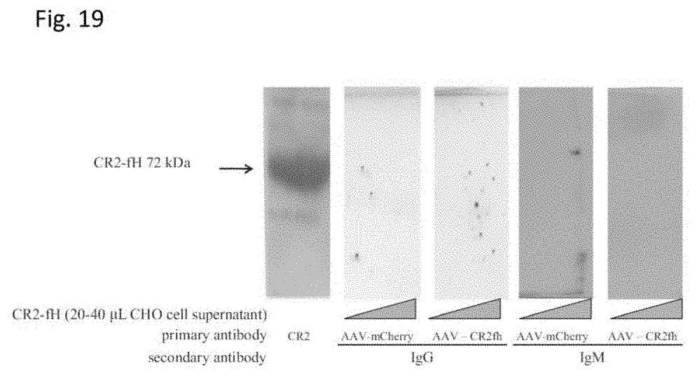

Administration or recombinant protein therapeutics can lend to the induction of anti-drug antibodies, which could interfere with the effect of the drug, or worse, could potentially cause tissue damage (CNV involves a natural antibody response). Since CR2-fH is a secreted protein, as opposed to mCherry, which is cytoplasmic, an assessment was made whether mice generate antibodies against CR2-fH one month after the injection; although no CR2-fH protein could be identified in mouse serum at that time point using conventional Western blotting. Using serum from experimental animals with AAV5-VMD2-CR2-fH when compared to controls (AAV5-VMD2-mCherry), neither IgG nor IgM antibodies recognizing CR2-fH could be identified (FIG. 19).

In summary, subretinal injections of AAV5-VMD2-CR2-mCherry or AAV5-VMD2-CR2-fH appeared to be safe and well tolerated, with the exception of the injection artifact.

### AAV5-VMD2-CR2-fH Reduces Complement Activation and Attenuates CNV Development

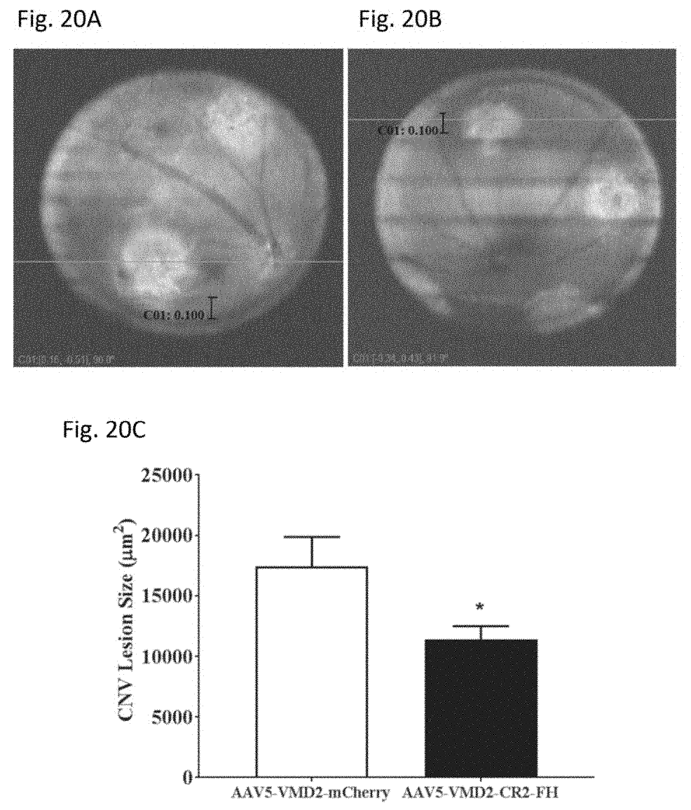

The effectiveness of AAV5-VMD2-CR2-fH treatment on CNV lesion size was evaluated 5 days following laser-induced photocoagulation. Mice were injected subretinally with either AAV5-VMD2-mCherry (control) or AAV-VMD2-CR2-fH. Mice were allowed to recover following the injection for 1 month and subretinal reattachment was confirmed by OCT and fundus photography. After reattachment (˜1 month), mice underwent laser-induced CNV in all four quadrants of the eye. A significant decrease (P≤0.05) in lesion size was observed in AAV5-VMD2-CR2-fH when compared to the control AAV5-VMD2-mCherry (FIG. 20). This decrease of ˜30% is comparable with previously published results using CR2-fH administered through intravenous injection (Rohrer et al., 2009).

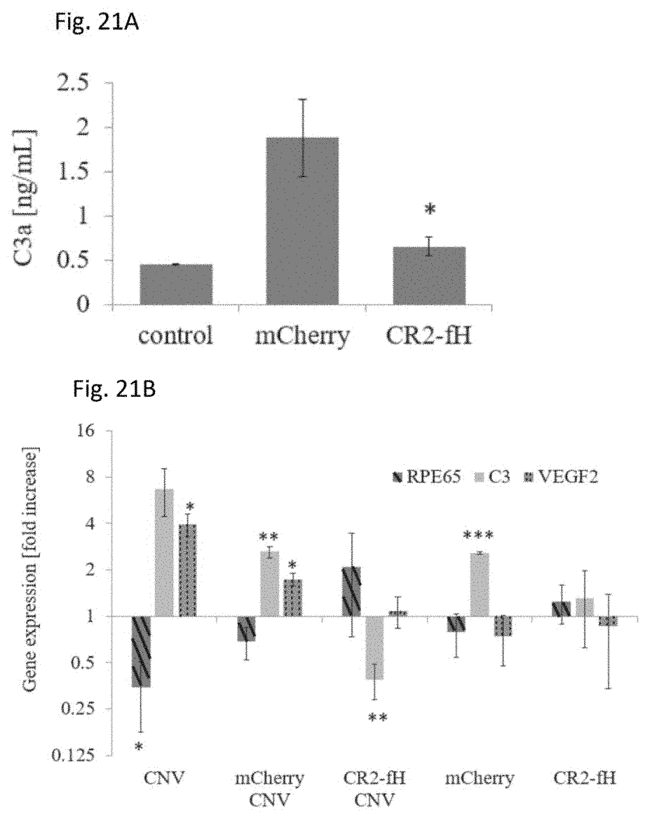

To confirm that CR2-fH acts by reducing complement activation, RPE/choroid fractions were assessed for C3a, the cleavage product of C3. ELISA measurements demonstrated that CNV (4 lesions per eye) resulted in a ˜4-fold increase in C3a when compared to naïve age-matched control eyes, an effect that was completely blocked by the presence of CR2-fH (FIG. 21A). Likewise, gene expression analysis for a subset of genes (RPE65 to assess RPE health; C3 to assess complement activation and VEGF to assess angiogenesis) revealed that the changes induced by CNV (reduction of RPE65 and increase in C3 and VEGF) were reversed by the expression of CR2-fH (FIG. 21B).

### Discussion

The goal of this study was to assess the use of AAV-mediated delivery of CR2-fH as a therapeutic strategy to reduce CNV in a murine model. The main results were: (1) the CD5 signal peptide enabled CR2-fH secretion from both the apical and basal side of the RPE when cells were transfectd with the PBM-CD5-CR2-fH vector: (2) a safe concentration of AAV5-VMD2-CR2-fH was identified based on structure function testing of the retina and RPE, and defined as a concentration in which effects of the injection were due to the injection artifact and retinal detachment and not the gene expressed (mCherry=CR2-fH); (3) an order of magnitude estimation suggests that similar amounts of CR2-fH are present in RPE/choroid samples with CNV when purified CR2-fH protein is provided by tail vein injection when compared to the expression levels produced by 3×10¹¹ vg/mL of AAV5-VMD2-CR2-fH; (4) CR2-fH expressed in the RPE was shown to reduce the development of CNV, prevent complement activation as determined by a reduction in C3a production and reverse CNV-associated changes in gene expression. AAV-vector-driven expression of complement inhibitors was efficacious in reducing local complement activation in the eye, and supports the feasibility of gene therapy as a potential treatment for AMD in humans.

### Complement Therapeutics in AMD

Complement inhibitors have been extensively evaluated in animal models of disease, as complement is involved in many human conditions (Ricklin et al., 2007). The complement system can be controlled both in fluid phase by soluble complement inhibitors, as well as on cell surfaces by membrane-bound inhibitors. Inhibitors can be further subdivided based on the process of the complement cascade they inhibit, whether they prevent activation of a particular pathway, or steps in the common terminal pathway. The best characterized complement inhibitors are a soluble form of complement receptor 1 (CR1) (Whiss, 2002) and an anti-C5 monoclonal antibody (mAb) (Kaplan, 2002). These inhibitors are systemically active inhibitors. Other preclinical complement inhibitors that are effective in animal models of human disease have been summarized (Ricklin et al., 2007). In the mouse model of CNV, anti-complement therapeutics targeting different steps in the cascade hove been found to be efficacious in reducing CNV, concomitant with a reduction in VEGF. Those include siRNA against CFB (Bora et al., 2006), the C3 convertase inhibitor compstatin Sun et al., 2012), antibodies against the anaphylatoxins C3a and C5a (Noznki et al., 2006), and membrane-targeted or non-membrane-targeted soluble CD59 (Bora et al., 2010; Cashman et al., 2011). Finally, in GA patients, an anti-CFD monoclonal antibody (lampalizumab) showed significant protection against the progression of GA in subgroups of patients Wright et al., 2016). These findings together with the genetic data, suggest AP activity as the common target in wet and dry AMD. Reducing AP would keep the classical and lectin intact, the two required pathways required to respond to infection. To reduce AP activity, strategies include to reduce activators (e.g., lampalizumab) or to increase inhibitors (such as CFH, Rohrer et al., 2009). In AMD, the most prevalent CFH variant is the Y402H polymorphism, which lies in the polyanion-binding domain of CFH (SCR 7) and appears to impair binding of CFH to BrM (Clark et al., 2006: Clark et al., 2010) or ligands such as malondinldehyde (Weismann et al., 2011). In addition, we have shown that oxidative stress impairs regulation at the RPE cell surface by CFH present within the serum (Thurman et al., 2009). Hence, a successful strategy to supply AP inhibition could focus on generating a CFH-like molecule (i.e., provide decay-acceleration and cofactor activity) while relying on an alternate strategy for membrane binding. Proof of principle was shown that CR2-fH, which relies entirely on the CR2 domain for targeting to sites of opsonin deposition, specifically targeted to sites of complement activation and injury in many different disease models, including the RPE/choroid of CNV animals, the RPE/Bruch's membrane in smoke-induced ocular pathology, as well as oxidatively-stressed RPE cells²⁴. These findings are now extended in this test of efficacy of CR2-fH provided by gene therapy.

### Anti-Complement Gene Therapy in AMD

The eye is an attractive organ for gene therapy, since it is easily accessible and immune privileged. Ever since the successful application of AAV gene therapy in Leber's Congenital Amauosis (reviewed in Pierce), novel methods and application in additional diseases or disease models are under development. Relevant for our studies targeting the complement system, CD59, an inhibitor for MAC has been shown to reduce murine CNV when provided either in the form of a soluble fusion protein, or by gene therapy. CD59-IgG20-fusion proteins injected into the vitreous or AAV2-mediated sCD59 gene expression injected either subretinally or intravitreally, all reduce CNV significantly. Soluble CD59 gene expression was driven by a chicken-beta-actin promoter (a total of 8×10⁹ genome copies of AAV) and its ability to act as an inhibitor was confirmed by analyzing MAC deposition in the CNV lesions. Here gene therapy vectors were used to deliver complement inhibitors locally within the eye, using the AAV5 vector, and ensuring tissue-specific expression using the VMD2 promoter. Efficacy for CR2-fH was noted in reducing complement activation and concomitant CNV (a total of 3×10⁸ genome copies of AAV was provided). In addition, this analysis was extended by carefully documenting the injections artifacts produced by the subretinal injection and the retinal detachment. Specifically, a reduction in the cone-driven OKR responses (spatial acuity and contrast sensitivity), a reduction in the RPE-driven c-wave of the full-field ERG, and a reduction in the focal ERG in regions close to the injection site where the RPE exhibited morphological irregularities were noted. However, in regions with normal RPE morphology and normal retinal thickness, focal ERG amplitudes were less impaired. Importantly, no gene-specific effects were observed, indicating that all of these functional and structural alterations were driven by the injection itself, and not by the gene expressed. And finally, despite the secretion of CR2-fH, no generation of anti-CR2-fH IgG or IgM antibodies was detected after one month of protein exposure.

While our experiments documented reduction of CNV and evidence of complement inhibition, functional and structural damage driven by the injection and retinal detachment were noted. Hence, gene expression, targeting cells in the inner retina should be considered such as reported for sCD59. Intravitreal injection of AAV2 driving soluble Flt-1 linked to IgG1-Fc using the chicken β-actin promoter have been reported for the treatment of advanced neovascular AMD (NCT01024998)². Specifically, in preclinical experiments intravitreal injections via transduction of some ganglion and transitional epithelial cells in the pars plana, provided long-term expression of sFLT1. And while injections in patients were well tolerated, long-term efficacy has not yet been provided. It is plausible that long-term inhibition of VEGF with sFLT1 results in resistance⁴⁰, or other compensatory effects such as an increase in complement activation⁴¹. These kinds of long-term compensatory effects are not expected with the CR2-fH proteins. CR2-fH has a short half-life in circulation and only bind to sites of injury, but not to healthy tissue²², thus it should not interfere with tissue homeostasis. Animals with 3-day old CNV lesions, the time point with maximal complement activation,¹⁵ were compared. Finally, with the development of new viral vectors that can penetrate the inner limiting membrane⁴², a larger array of cells can presumably be targeted, providing more options for gene delivery.

In summary, we provided evidence that a targeted complement inhibitor can be used to provide effective complement inhibition in the eye, reducing AP of complement-dependent CNV progression. We are currently investigating feasibility of long-term complement inhibition in the smoke-induced ocular pathology model¹⁶. Inhibiting the AP of complement reduces overall complement activation, but retaining normal levels of complement required for cellular homeostasis. Finally, demonstration of efficacy using AAV-vectors opens up new avenues for the development of treatment strategies in age-related macular degeneration and other complement-dependent diseases.

### Materials and Methods

### Adeno-Associated Virus Construct

The plasmid construct of CR2-fH was previously described¹⁵. In short, it contains the sequence encoding the 4 N-terminal SCRs of mouse CR2 (residues 1-257 of mature protein, RefSeq accession number M35684) followed by the 5 N-terminal SCR's of mouse fH (residues 1-303 of mature protein, RefSeq NM009888), interspaced with a (G₄S)₂ linker. The expression plasmid was the previously described PBM vector with a CD5 signal peptide sequence required for secretion⁴³. The plasmid construct was transfected into ARPE-19 cells with FuGene HD transfection reagent according to the manufacturer's instructions (Roche Applied Science, Indianapolis, Ind.), and protein secretion into the apical and basal compartment monitored in polarized RPE cells. CR2-fH gave a single band of appropriate molecular weight by SDS-PAGE.

After confirmation of polarized secretion of CR2-fH from RPE cells, the CR2-fH sequence was used to generate the AAV5-VMD2-CR2-fH vector. The same vector backbone (AAV5-VMD2) was used to generate AAV5-VMD2-mCherry⁴⁴.

### Viral Vector Injection

All animal experiments were performed in accordance with the ARVO Statement for the Use of Animals in Ophthalmic and Vision Research and were approved by the University Animal Care and Use Committee. C57BL/6J mice (Jackson Laboratory, Bar Harbor, Me.) were generated from a colony within MUSC, to obtain mice always raised within the same microenvironment. Subretinal injections were performed using the trans-cornea route on mice 8-10 weeks of age under direct observation (dissecting microscope at 14×magnification) according to published protocols^{45, 46}. In short, mice were anesthetized by intraperitoneal injection (xylazine and ketamine, 20 and 80 mg/kg, respectively), their pupils dilated (phenylephrine HCL, 2.5% and atropine sulfate, 1%), and the ocular surface anesthetized (proparacaine hydrochloride) and lubricated (hydroxypropyl methylcellulose, 2.5%). An aperture through the superior cornea was generated (30½-gauge disposal needle) through which a 33-gauge unbeveled blunt needle mounted on a 2.5-mL Hamilton syringe (Hamilton Co., Reno, Nev. USA) was inserted to reach the subretinal space. One μL of vector suspension (in PBS) with 1% fluorescein as an indicator dye was slowly injected, with subretinal blebs formation indicating success. Retinal detachment was confirmed by optical coherence tomography (OCT) and fundus photography, and size and location documented. In preliminary experiments, viral concentrations ranging from 1 mL of 1×10¹³ viral genome (vg) containing particles/mL (representing a dose we found to successfully restore vision in rd12 mice⁴⁷) to 1 μL of 1×10¹⁰ vg/mL were tested, comparing AAV5-VMD2-CR2-M and AAV5-VMD2-mCherry. Expression and secretion of CR2-fH driven by 1×10¹³ vg/mL was found to reduce the ERG response 1 month other the injection when compared to equal an concentration of AAV5-VMD2-mCherry; a concentration of 3×10¹¹ vg/mL of AAV5-VMD2-CR2-fH was found to be both efficacious and safe (see below). Further analysis was carried out only on mice with successful retinal detachments as well as successful reattachments at 1 month post injection as confirmed by fundus photography and OCT.

### Choroidal Neovascularization

Following reattachment of the retina, at approximately 1 month, argon laser photocoagulation (532 nm, 100 μm spot size, 0.1 s duration, 100 mW) was used to generate 4 laser spots around the optic nerve of each eye¹⁵. As previously described, a bubble formation at the site of the laser burn was used to determine Bruch's membrane rupture⁴⁸. Five days following laser-induced CNV, mouse eyes were imaged by OCT and fundus photography before being sacrificed for tissue collection on day 6.

### Dot Blot and Western Analysts

Retina and mouse RPE/choroid was collected from CNV-lesion mice injected with AAV5-VMD2-CR2-fH, AAV5-VMD2-mCherry or soluble CR2-fH (250 μg, via tailvein injection on day 3 after CNV induction, and collected 24 hours later). Protein was extracted by first solubilizing in R1PA buffer (10 mM Tris-Hcl, pH 7.5. 300 mM NaCl, 1 mM EDTA, 1% Triton X-100, 1% SDS, and 0.1% sodium deoxycholate; Thermo Fisher Scientific, Waltham, Mass.) containing protease inhibitor cocktail (Sigma-Aldrich, St. Louis, Mo.). Whole tissue lysates were collected following centrifugation (20000×g for 30 min at 4° C.) and total protein (25 μL) was loaded directly into the wells of a 96 well plate. Using the BIO_DOT® Microfiltration Apparatus (Bio-Rad Laboratories Inc. Hercules, Calif.), samples were transferred onto a nitrocellulose membrane. The dotted membrane was then rinsed with TBST wash buffer before being blocked for 2 hours at room temperature with 5% nonfat milk in TBST buffer. CR2-fH was detected using an anti-CR2 primary antibody (10 μg/mL; rat anti-mouse CD21, clone 7G6; purified in house⁴³) incubated in 5% nonfat milk/TBST (1:1000) overnight, and visualized with a horseradish peroxidase-conjugated secondary antibody (anti-rat; Santa Cruz Biotechnology, Inc. Dallas, Tex.) followed by incubation with Clarity™ Western ECL Blotting Substrate (Bio-Rad Laboratories Inc.). For Western blot analysis, supernatant from CR2-fH-expressing cultured cells were added to Laemmli sample buffer and boiled. Samples were separated by electrophoresis on a 4-20% CRITERION™ TGX™ Precast Gels (Bio-Rad Laboratories, Inc.), and proteins transferred to a PVDF membrane. Membranes were incubated with primary antibody against CR2, or scrum (1:50) from mice treated with subretinal AAV5-VMD2-CR2-fH or AAV5-VMD2-mCherry vectors. Proteins were visualized with horseradish peroxidase-conjugated secondary antibodies (anti-mouse IgG and IgM; Santa Cruz Biotechnology) followed by incubation with Clarity™ Western ECL Blotting Substrate (Bio-Rad Laboratories, Inc.) and chemiluminescent detection. Protein bands or dots were scanned and densities quantified using ImageJ software.

### Immunohistochemistry

Eyecups were collected as previously described, with the lens, anterior and retinas removed¹⁵, fixed overnight with 4% paraformaldehyde (PFA) and washed in PBS. Eyecups were incubated in blocking solution (10% normal goal serum and 0.4% Trilan-X in tris-buffered saline) containing polyclonal ZO-1 antibody (1:200; Cat# 61-7300, Invitrogen) or anti-CR2 primary antibody. Eyecups were washed following incubation with a secondary antibody (Alexa Fluor 488 goal-anti-rabbit; 1:500: Cat# A-1008, Invitrogen; Alexa Fluor 488 goat-anti-mouse; 1:500: Cat# A-11008, Invitrogen). In addition, cells were stained with Alexa Fluor 594 phalloidin (diluted in PBS 1:40: Cat# 12381, Invitrogen). Eyecups were then flattened onto a glass slide using four relaxing cuts, mounted with a coverslip using Fluoromount (Southern Biotechnology Associates, Inc., Birmingham, Ala.) and examined with fluorescence microscopy (Zeiss, Thornwood, N.Y.) equipped with a digital black-and-white camera (Spot camera: Diagnostic Instruments, Sterling Heights, Mich.).

### Optokinetic Response Test (OKR)

Visual Acuity and Contrast Sensitivity were measured using the OptoMotry software as previously described^{49,50}. Mice were placed on an elevated pedestal placed in the center of four computer monitors displaying stimulus gratings. Following a two minute adjustment period, optomoter responses were measured by observation of the mouse head response following the direction of a rotating vertical grating, through overhead closed-circuit TV cameras. Using a constant speed of 12 deg/s, and 100% contrast, visual acuity was determined by observing the animal's response to spatial frequency display by means of a staircase procedure. By taking the reciprocal of the contrast threshold at 0.131 cyc/deg and a speed of 12 deg/s, contrast sensitivity was determined. All tests were conducted under a mean luminance of 532 cd m⁻².

### Full Field and Focal Electroretinography

Mice were dark-adapted overnight, anesthetized with xylazine and ketamine (20 and 80 mg/kg, respectively), and pupils dilated with phenylephrine HCl (2.5%) and atropine (1%). A drop of lubricant eyedrops (Goniovisc, Rancho Cucamonga, Calif.) was applied to foster the electrical contact between the electrode and the cornea. Needle electrodes inserted into the scalp and tail provided reference and ground, respectively.

Electrical responses of the RPE (c-waves) were analyzed using full field electroretinography (ERG) using a setup previously described⁵⁰. In short, c-waves were recorded using a UTAS E-4000 System (LKC Technologies, Gaithersburg, Md.) in response to a flash at 100 cd*s mm^{-2 51}, using corneal loop electrodes. The amplitude of the c-wave was measured from the baseline to the maximum of the peak. Recordings were obtained at baseline prior to as well as 1 month following subretinal injections.

Focal ERGs (fERGs) were recorded using the image guided Micron III focal ERG system (Phoenix Research labs, USA) using the corneal electrodes integrated into the lens mount. A spot size of 0.5 mm was selected to measure fERGs within and away from the detached retinal area. Voltage traces were recorded in response to three different flash strengths (3.2, 5 and 6.8 cd m²) at a duration of 2 ms, and analyzed with LabScribe software (Version 3.015200) that comes standard with the fERG system. Ten sweeps were averaged for the low flash strength, 5 sweeps for the higher flash strengths, however, other sweeps where the software could not properly identify the trough of the a-wave or the peak of the b-wave were manually excluded from the calculations.

### RPE Morphology Assessment

The CellProfiler V2.11 software (http://vww.cellprofiler.org) was used to evaluate RPE morphology as previously described⁵². TIF files of equal size images and exposure time were first imported into the software and analyzed using a customizable script. Cells were compared using the pipeline “neighboring cells” from which the form factor (where a perfectly circular object equals 1) and eccentricity (the degree measured between 0 and 1, to which an object represent an ellipse) were obtained. Morphology of RPE cells was analyzed within the peri-lesion area of the injection site and compared to the tiling pattern of an area (45*74 μm, depth by width) surrounding the peri-lesion. Morphology measurements for age-matched untreated CS7BL/6J mice were obtained for reference purposes.

### Optical Coherence Tomography

Optical coherence tomography (OCT) was used to quantify retinal thickness⁵⁰ and analyze CNV lesion size on day 5 post laser treatment as previously described⁵³⁻⁵⁵. Mice were anesthetized prior to imaging and eyes kept hydrated with normal saline. Using an SD-OCT BIOOPTIGEN® Spectral Domain Ophthalmic Imaging System (Bioptigen Inc., Durham N.C.), the eyes were imaged.

To assess retina structure, rectangular volume scans were taken in the nasal quadrant from the optic disc, each volume consisting of 33 B-scans (1,000 A-scans per B-scan). Five separate scans were collected and averaged to generate a high resolution image. Vertical calipers were placed to measure thickness of the different retinal layers for each scan. All measurements were taken 500 μm from the optic disc⁵⁰.

For CNV lesion analyses, rectangular volume scans images set at 1.6×1.6 mm. consisting of 100 B-scans (1000 A-scans per B scan) were squired. Using methods previously described by Giani and colleagues, the cross-sectional area of the lesion was measured by using the en-face fundus reconstruction tool to ascertain the midline passing through the RPE-Bruch's membrane rupture with the axial interval positioned at the level of the RPE/choroid complex⁵⁶. Image J software (Wayne Rasband, National Institutes of Health, Bethesda, Md.: available at <http://rsb.info.nih.gov/ij/index.html≥) was used to measure the area around the hyporeflective spot produced on the fundus image, with vertical calipers set at 0.100 mm at the site of each lesion. Based on the size of the individual pixels (1.6×1.6 μm), the lesion sizes were calculated.

### C3a ELISA

C3a levels were measured in RPE/choroid fractions of mice treated by subretinal injection with either AAV5-VMD2-mCherry or AAV5-VMD2-CR2-fH prior to CNV, as well as control eyes using the Mouse Complement C3 ELISA from LifeSpan Biosciences, Inc. (Seattle, Wash.). RPE/choroid tissues were quickly prepared on ice by rinsing with ice cold PBS to remove excess blood. RPE/choroid cells were then lysed by ultrasonication using ice cold PBS. Centrifugation of the final homogenate was performed at 5,000 G for 5 minutes before continuing with the assay procedure as described in the manufacturer's protocol. Final values were read using a microplate reader set at 450 nm.

### Quantitative RT-PCR

Retina and RPE/choroid fractions of the mouse eye were isolated and stored at −80° C. until use. Using the miRNeasy Kit (Qiagen, Valencia, Calif.) total RNA was isolated and purified and RNA with a 260/280 ratio of 1.95-2.1 (Take 3 Micro-Volume Plates, Biotek, Winooski, Vt.) was used to generate first strand cDNA (Qiagen). PCR amplifications were performed in triplicate as previously described⁵⁰ using the Realplex 2 Mastercycler (Eppendorf, Hauppauge, N.Y.). Printers sequences for each gene product are listed in Table 1. Cycle number (Ct value), was used to obtain quantitative values as previously described⁵⁷ with genes of interest normalized to β-actin. Using the Student t-test, fold differences between AAV5-VMD2-fH and AAV5-mCherry in the presence or absence of CNV (control) were determined.

### Statistics

Data are presented as mean±SEM. Single comparisons were analyzed using unpaired t-tests, with mean value differences considered significant at P≤0.05. For data consisting of multiple groups and repealed measures, repeated measure ANOVA was used.

TABLE 1
Quantitative RT-PCR primer sequences.

| Gene Name | Symbol | Forward Primer | Reverse Primer |
|---|---|---|---|
| retinal pigment | Rpe-65 | 5′-TTCTGAGTGTGGTGGTGAGC- | 5′-AGTCCATGGAAGGTCACAGG- |
| epithelium 65 | | 3′ (SEQ ID NO: 29) | 3′ (SEQ ID NO: 30) |
| complement | C3 | 5′-TCAGATAAGGAGGGGCACAA- | 5′-ATGAAGAGGTACCCACTCTGGA- |
| component 3 | | 3′ (SEQ ID NO: 31) | 3′ (SEQ ID NO: 32) |
| vascular | Vegfa | 5′-AGCACAGCAGATGTGAATGC- | 5′-TTTCTTGCGCTTTCGTTTTT-3′ |
| endothelial | | 3′ (SEQ ID NO: 33) | (SEQ ID NO: 34) |
| growth factor A | | | |
| actin, beta | Actb | 5′-AGCTGAGAGGGAAATCGTGC- | 5′-ACCAGACAGCACTGTGTTG-3′ |
| | | 3′ (SEQ ID NO: 35) | (SEQ ID NO: 36) |

### CITED REFERENCES

1. Pierce, E A, and Bennett, J (2015). The Status of RPE65 Gene Therapy Trials: Safety and Efficacy. Cold Spring Harb Perspect Med 5: a017285.
2. Heier, J S, Kherani, S, Desai, S, Dugel, P, Kaushal, S, Cheng, S H, et al. (2017). Intravitreous injection of AAV2-sFLT01 in patients with advanced neovascular age-related macular degeneration: a phase 1, open-label trial. Lancet, 390(10089):50-61.
3. Han, Z, Conley, S M, Makkia, R S, Cooper, M J, and Naash, M I (2012). DNA nanoparticle-mediated ABCA4 delivery rescues Stargardt dystrophy in mice. J. Clin Invest. 122: 3221-3226.
4. Li, M, Xu, J, Chen, X, and Sun, X (2009). RNA interference as a gene silencing therapy for mutant MYOC protein in primary open angle glaucoma. Diagn Pathol. 4: 46.
5. Yates, J R, Sepp, T, Matharu, B K, Khan, J C, Thurlby, D A, Shahid, H, et al. (2007). Complement C3 variant and the risk of age-related macular degeneration. N. Engl. J. Med. 357: 553-561.
6. Gold, B, Merriam, J E, Zernant, J, Hancox, L S, Taiber, A J, Gehrs, K, et al. (2006). Variation in factor B (BF) and complement component 2 (C2) genes is associated with age-related macular degeneration. Nat. Genet. 38: 458-462.
7. Seddon, J M, Yu, Y, Miller, E C, Reynolds, R, Tan, P L, Gowrisankar, S, et al. (2013). Rare variants in CF1, C3 and C9 are associated with high risk of advanced age-related macular degeneration. Nat Genet 45: 1366-1370.
8. Fagerness, J A, Maller, J B, Neale, B M, Reynolds, R C, Daly, M J, and Seddon, J M (2009). Variation near complement factor 1 is associated with risk of advanced AMD. Eur J Hum Genet 17: 100-104.
9. Fritsche, L G, Igl, W, Bailey, J N, Grassmann, F, Sengupta, S, Bragg-Gresham, J L, et al. (2016). A large genome-wide association study of age-related macular degeneration highlights contributions of rare and common variants. Nat Genet 48: 134-143.
10. Tan, P L, Bowes Rickman, C, and Katsanis, N (2016). AMD and the alternative complement pathway: genetics and functional implications. Hum Genomics 10: 23.
11. Muller-Ebechard, H J (1988). Molecular organization and function of the complement system. Annual Review of Biochemistry 57: 321-347.
12. Pangburn, M K, Rawal, N, Cortes, C, Alam, M N, Ferreira, V P, and Atkinson, J A (2009). Polyanion-induced self-association of complement factor H. J. Immunol. 182:1061-1068.
13. Kim, Y H, He, S, Kase, S, Kitamura, M, Ryan, S J, and Hinton, D R (2009). Regulated secretion of comp ement factor H by RPE and its role in RPE migration. Graefe's archive for clinical and experimental ophthalmology=Alhrecht van Graefes Archiv fur klinische und experimentelle Ophthalmologic 247:651-659.
14. Rodriguez, E, Rallapalli, P M, Osborne, A J, and Perkins, S J (2014). New functional and structural insights from updated mutational databases for complement factor H. Factor I, membrane cofactor protein and C3. Biosci Rep 34(5).
15. Rohrer, B, Long, Q, Coughlin, B, Wilson, R B, Huang, Y, Qiao, F, et al. (2009). A targeted inhibitor of the alternative complement pathway reduces angiogenesis in a mouse model of age-related macular degeneration. Invest. Ophthalmol. Vis Sci 50: 3056-3064.
16. Woodell, A, Jones, B W, Williamson, T, Schnabolk, G, Toralinson, S, Atkinson, C, et al. (2016). A Targeted Inhibitor of the Alternative Complement Pathway Accelerates Recovery From Smoke-Induced Ocular Injury. Invest Ophthalmol Vis Sci 57: 1728-1737.
17. Holers, V M, Rohrer, B, and Tomlinson, S (2013). CR2-mediated targeting of complement inhibitors: bench-to-bedside using a novel strategy for site-specific complement modulation. Adv. In Exper. Med. and Biology 735: 137-154.
18. Cashman, S M, Gracias, J, Adhi, M, and Kumar-Singh, R (2015). Adenovirus-mediated delivery of Factor H attenuates complement C3 induced pathology in the murine retina: a potential gene therapy for age-related macular degeneration. J. Gene Med. 17:229-243.
19. Pang, J J, Lauramore, A, Deng, W T, Li, Q, Doyle, T J, Chiodo, V, et al. (2008). Comparative analysis of in vivo and in vitro AAV vector transduction in the neonatal mouse retina: effects of serotype and site of administration. Vision Research 48: 377-385.
20. Kong, F, Li, W, Li, X, Zheng, Q, Dai, X, Zhou, X, et al. (2010). Self-complementary AAV5 vector facilitates quicker transgene expression in photoreceptor and retinal pigment epithelial cells of normal mouse. Experimental Eye Research 90: 546-554.
21. Esumi, N, Oshima, Y, Li, Y, Campochiaro, P A, and Zack, D J (2004). Analysis of the VMD2 promoter and implication of E-box binding factors in it regulation. J. Biol Chem. 279: 19064-19073.
22. Alawieh, A, and Tomlinson, S (2016). Injury site-specific targeting of complement inhibitors for treating stroke. Immunol Rev 274: 270-280.
23. Remtulla, S, and Hallett, P E (1985). A schematic eye for the mouse, and comparisons with the rat. Vision Research 25: 21-31.
24. Joseph, K, Kulik, L, Coughlin, B, Kunchithapautham, K, Bandyopadhyay, M, Thiel, S, et al. (2013). Oxidative Stress Sensitizes RPE Cells to Complement-Mediated Injury in a Natural Antibody-, Lectin Pathway- and Phospholipid Epitope-Dependent Manner. J Biol Chem. 288(18): 12753-65.
25. Ricklin, D, and Lambris, J D (2007). Complement-targeted therapeutics. Nat Biotechnol 25:1265-1275.
26. Whiss, P A (2002). Pexelizumab Alexion. Curr Opin Investig Drugs 3: 870-877.
27. Kaplan, M (2002). Eculizumab (Alexion). Curr Opin Investig Drugs 3: 1017-1023.
28. Bora, N S, Kaliappan, S, Jha, P, Xu, Q, Sohn, J H, Dhaulakhandi, D B, et al. (2006). Complement activation via alternative pathway is critical in the development of laser-induced choroidal neovascularization: role of factor B and factor H. J Immunol 177: 1872-1878.
29. Sun, Y Yu, W, Huang, L, Hou, J, Gong, P, Zheng, Y, et al. (2012). Is asthma related to choroidal neovascularization! PLoS ONE 7: e35415.
30. Nozaki, M, Raisler, B J, Sakura, E, Sarma, J V, Barnum, S R, Lambris, J D, et al. (2006). Drusen complement components C3a and C5a promote choroidal neovascularization. Proc Natl Acad Sci USA 103: 2328-2333.
31. Bora, N S, Jha, P, Lyzogubov, V V, Kaliappan, S, Liu, J, Tytarenko, R G, et al. (2010). Recombinant membrane-targeted form of CD59 inhibits the growth of choroidal neovascular complex in mice. J. Biol. Chem. 285: 33826-33833.
32. Cashman, S M, Ramo, K, and Kumar-Singh, R (2011). A non membrane-targeted human soluble CD59 attenuates choroidal neovascularization in a model of age related macular degeneration. PloS one 6; e19078.
33. Wright, C B, and Ambati, J (2016). Dry Age-Related Macular Degeneration Pharmacology. Handb Exp Pharmacol.
34. Clark, S J, Higman, V A, Mulloy, B, Perkins, S J, Lea, S M, Sim, R B, et al. (2006). His-384 allotypic variant of factor H associated with age-related macular degeneration has different heparin binding properties from the non-disease-associated form. J Biol Chem 281: 24713-24720.
35. Clark, S J, Perveen, R, Hakobyan, S, Morgan, B P, Sim, R B, Bishop, P N, et al. (2010). Impaired binding of the age-related macular degeneration-associated complement factor H 402H allotype to Bruch's membrane in human retina. J Biol Chem 285: 30192-30202.
36. Weismann, D, Hartvigsen, K, Lauer, N, Bennett, K L, Scholl, H P, Charbel Issa, P, et al. (2011). Complement factor H binds malondialdehyde epitopes and protects from oxidative stress. Nature 478: 76-81.
37. Thurman, J M, Renner, B, Kunchithapautham, K, Ferreira, V P, Pangburn, M K, Ablonczy, Z, et al. (2009). Oxidative stress renders retinal pigment epithelial cells susceptible to complement-mediated injury. J. Biol. Chem. 284: 16939-16947.
38. Liu, M M, Tuo, J, and Chan, C C (2011). Republished review: Gene therapy for ocular diseases. Postgrad Med J 87: 487-495.
39. Adhi, M, Cashman, S M, and Kumar-Singh, R (2013). Adeno-associated virus mediated delivery of a non-membrane targeted human soluble CD59 attenuates some aspects of diabetic retinopathy in mice. PloS One 8: e79661.
40. Yang, S, Zhao, J, and Sun, X (2016). Resistance to anti-VEGF therapy in neovascular age-related macular degeneration: a comprehensive review. Drug Des Devel Ther 10: 1857-1867.
41. Keir, L S, Firth, R, Aponik, L, Feitelberg, D, Sakimoto, S, Aguilar, E, et al. (2017). VEGF regulates local inhibitory complement proteins in the eye and kidney. J Clin Invest 127: 199-214.
42. Boyd, R F, Sledge, D G, Boye, S L, Boye, S E, Hauswirth, W W, Komaromy, A M, et al. (2016). Photoreceptor-targeted gene delivery using intravitreally administered AAV vectors in dogs. Gene Invest. 23: 223-230.
43. Song, H, He, C, Knaak, C, Guthridge, J M, Holers, V M, and Tomlinson, S (2003). Complement receptor 2-mediated targeting of complement inhibitors to sites of complement activation. J. Clin. Invest. 111: 1875-1885.
44. Ryals, R C, Boye, S L, Dinculescu, A Hauswirth, W W, and Boye, S E (2011). Quantifying transduction efficiencies of unmodified and tyrosine capsid mutant AAV vectors in vitro using two ocular cell lines. Molecular Vision 17: 1090-1102.
45. Pang, J J, Boye, S L, Kumar, A, Dinculescu, A, Deng, W, Li, J, et al. (2008). AAV-mediated gene therapy for retinal degeneration in the rd10 mouse containing a recessive PDEbeta mutation. Invest Opthalmol Vis. Sci. 49: 4278-1283.
46. Pang, J J, Boye, S E, Lei, B, Boye, S L, Everhart, D, Ryals, R, et al. (2010). Self-complementary AAV-mediated gene therapy restores cone function and prevents cone degeneration in two models of Rpe65 deficiency. Gene Ther. 17(7):815-26.
47. Pang, J J, Chang, B, Hawes, N L, Hurd, R E, Davisson, M T, Li, J, et al. (2005). Retinal degeneration 12 (rd12): a new, spontaneously arising mouse model for human Leber congenital amaurosis (LCA). Mol Vis 11: 152-162.
48. Nozaki, M, Raisle, B J, Sakurai, E, Samra, J V, Barnum, S R, Lambris, J D, et al. (2006). Drusen complement components C3a and C5a promote choroidal neovascularization. Proc. Nat. Acad Sci. USA 103: 2328-2333.
49. Prusky, G T, Alam, N M, Beekman, S, and Douglas, R M (2004). Rapid quantification of adult and developing mouse spatial vision using a virtual optomotor system. Invest Ophthalmol Vis Sci. 45:4611-4616.
50. Woodell, A, Conghlin, B, Kunchithapautham, K, Casey, S, Williamson, T, Ferrell, W D, et al. (2013). Alternative complement pathway deficiency ameliorates chronic smoke-induced functional and morphological ocular injury. PloS one 8: e67894.
51. Ablonczy, Z, Dahrouj, M, and Marneros, A G (2014). Progressive dysfunction of the retinal pigment epithelium and retina due to increased VEGF-A levels. FASEB J 28: 2369-2379.
52. Obert, E, Strauss, R, Brandon, C, Grek, C, Ghatnekar, G, Gourdie, R, et al. (2017). Targeting the tight junction protein, zonula occluders-1, with the connexin43 mimetic peptide, alphaCT1, reduces VEGF-dependent RPE pathophysiology. J. Mol. Med. (Berlin, Germany).
53. Schnabolk, G, Coughlin, B, Joseph, K, Kunchithapautham, K, Bandyopadhyay, M, O'Quinn, E, et al. (2015). Local production of the alternative pathway component, Factor B, is sufficient to promote laser-induced choroidal neovascularization. Invest Ophthalmol Vis Sci. 56(3): 1850-1863.
54. Schnabolk, G, Stauffer, K, O'Quinn, E, Coughlin, B, Kunchithapautham, K, and Rohrer, B (2014). A comparative analysis of C57BL/6J and 6N substrains; chemokine/cytokine expression and susceptibility to laser-induced choroidal neovascularization. Exp. Eye Research 129: 18-23.
55. Coughlin, B, Schnabolk, G, Joseph, K, Raikwar, H, Kunchithapautham, K, Johnson, K, et al. (2016). Connecting the innate and adaptive immune responses in mouse choroidal neovascularization via the anophylatoxin C5a and gammadeltaT-cells. Scientific Reports 6: 23794.
56. Giani, A, Thanos, A, Roh, M I, Connolly, E, Trichoma, G, Kim, I, et al. (2011). In vivo evaluation of laser-induced choroidal neovascularization using spectral-domain optical coherence tomography. Invest Ophthalmol Vis Sci. 52: 3880-3887.
57. Lohr, H R, Kuntchithapautham, K, Sharma, A K, and Rohrer, H (2006). Multiple, parallel cellular suicide mechanisms participate in photoreceptor cell death. Exp Eye Res 83: 380-389.

Although the foregoing compounds, compositions and methods have been described in some detail by way of illustration and example for purposes of clarity of understanding, it is apparent to those skilled in the art that certain minor changes and modifications will be practiced. Therefore, the description and examples should not be construed as limiting the scope of the disclosure.

The disclosures of all publications, patents, patent applications and published patent applications referred to herein are hereby incorporated herein by reference in their entirety.

What is claimed is:

1. A method of treating a subject at risk for or afflicted with glaucoma or macular degeneration, the method comprising administering to the subject an effective amount of a recombinant adeno-associated virus (AAV) vector operably encoding a complement receptor 2 (CR2)-factor H (FH) fusion protein; wherein said CR2-FH fusion protein comprises a CR2 portion comprising a CR2 or a fragment thereof, and a FH portion comprising a FH or a fragment thereof, said FH or fragment thereof comprising at least the first four N-terminal short consensus repeat (SCR) domains of FH; wherein the CR2 portion of the CR2-FH fusion protein is capable of binding to a CR2 ligand; and wherein the FH portion of the CR2-FH fusion protein is capable of inhibiting complement activation of the alternative pathway.

2. The method of claim 1, wherein the recombinant adeno-associated virus vector is a recombinant AAV2 vector or a recombinant AAV5 vector.

3. The method of claim 1, wherein the recombinant AAV vector comprises a chicken β-actin (CBA) promoter or a tissue-specific promoter for use in eye tissue.

4. The method of claim 3, wherein the eye tissue comprises retinal pigment epithelium (RPE) or choriod.

5. The method of claim 3, wherein the tissue-specific promoter comprises a VMD2 promoter.

6. The method of claim 1, wherein the recombinant AAV vector is administered to an eye of the subject using intravitreal or subretinal injection.

7. The method of claim 1, wherein the subject is at risk for or afflicted with glaucoma, and wherein administration of the recombinant AAV vector
slows, halts, or reverses the loss of retinal ganglion cells (RGC) in the subject;
slows, halts, or reverses the loss of Brn3+ retinal ganglion cells (RGC) in the subject;
slows, halts, or reverses intraretinal axon degeneration in the subject;
slows, halts, or reverses damage to optic nerves of the subject; and/or
slows, halts, or reverses the progression of glaucoma in the subject.

8. The method of claim 7, wherein the subject is at risk for or afflicted with macular degeneration, and wherein administration of the recombinant AAV vector is followed by reattachment of the retina in the subject.

9. The method of claim 1, wherein the subject is at risk for or afflicted with macular degeneration, and wherein administration of the recombinant AAV vector
slows, halts, or reverses choroidal neovascularization (CNV) in the subject;
reduces CNV-mediated complement activation; and/or
slows or halts the progression of macular degeneration in the subject.

10. The method claim 1, wherein the macular degeneration comprises age-related macular degeneration (AMD).

11. The method of claim 1, wherein the recombinant AAV vector comprises a nucleotide sequence that encodes a fusion protein having SEQ ID NO:21.

12. The method of claim 1, wherein the recombinant AAV vector comprises a nucleotide sequence that encodes a fusion protein fused to a CD5 or CR2 signal peptide.

13. The method of claim 1, wherein the recombinant AAV vector comprises SEQ ID NO: 22.

14. A recombinant adeno-associated virus (AAV) vector operably encoding a complement receptor 2 (CR2)-factor H (FH) fusion protein; wherein said CR2-FH fusion protein comprises a CR2 portion comprising a CR2 or a fragment thereof, and a FH portion comprising a FH or a fragment thereof, said FH or fragment thereof comprising at least the first four N-terminal short consensus repeat (SCR) domains of FH; wherein the CR2 portion of the CR2-FH fusion protein is capable of binding to a CR2 ligand; and wherein the FH portion of the CR2-FH fusion protein is capable of inhibiting complement activation of the alternative pathway.

15. The recombinant AAV vector of claim 14 comprising a recombinant AAV2 vector or a recombinant AAV5 vector.

16. The recombinant AAV vector of claim 14 comprising a chicken β-actin (CBA) promoter or a VMD2 promoter.

17. The recombinant AAV vector of claim 14 comprising a nucleotide sequence that encodes a fusion protein having SEQ ID NO: 21.

18. The recombinant AAV vector of claim 14 comprising SEQ ID NO: 22.

19. The recombinant AAV vector of claim 14 comprising a VMD2 promoter operably linked to a nucleic acid sequence encoding the complement receptor 2 (CR2)-factor H (FH) fusion protein.

20. A pharmaceutical composition comprising the recombinant adeno-associated virus (AAV) vector of claim 14.
